(19) United States
(12) Patent Application Publication
(10) Pub. No.: US 2023/0256435 A1
(43) Pub. Date: Aug. 17, 2023

(54) APPARATUSES AND METHODS FOR ANALYZING LIVE CELLS

(71) Applicant: AGILENT TECHNOLOGIES, INC., Santa Clara, CA (US)

(72) Inventors: Andrew C. Neilson, Sunapee, NH (US); Jason Dell'Arciprete, Santa Clara, CA (US); Eric John Schultz, North Andover, MA (US); Brady Robert Paradis, Santa Clara, CA (US); George W. Rogers, San Diego, CA (US); Yoonseok Kam, Lexington, MA (US)

(21) Appl. No.: 18/164,422

(22) Filed: Feb. 3, 2023

Related U.S. Application Data
(60) Provisional application No. 63/306,142, filed on Feb. 3, 2022.
(60) Provisional application No. 63/311,838, filed on Feb. 18, 2022.

(51) Int. Cl. B01L 3/00

(52) U.S. Cl. CPC B01L 3/502715; B01L 2400/0487; B01L 2200/10; B01L 2200/147

(57) ABSTRACT

An analytical instrument having a sensing system, a stage adapted to receive a sample carrier, a motion actuator assembly for positioning the sensing system and/or the stage, a temperature control element, and a signal processing module are disclosed. Methods of analyzing cell samples with the analytical instrument are also disclosed.

### CROSS REFERENCE TO RELATED APPLICATIONS

This application claims the benefit of U.S. Provisional Application No. 63/306,142, filed Feb. 3, 2022, and U.S. Provisional Application No. 63/311,838, filed Feb. 18, 2022. The contents of the aforementioned application are hereby incorporated by reference in its entirety.

### FIELD OF TECHNOLOGY

Aspects and embodiments disclosed herein relate generally to the measurement of the constituents (analytes) of an extracellular medium surrounding living cells.

### BACKGROUND

Oxygen consumption rate (OCR) and extracellular acidification rate (ECAR) rates are key indicators of mitochondrial respiration and glycolysis and these measurements provide a systems-level view of cellular metabolic function in cultured cells and ex vivo samples.

Despite the advancement of technology in this area, there exists a need for developing new apparatuses and methods for analyzing live cells.

### SUMMARY

In accordance with one aspect, there is provided an analytical instrument. The analytical instrument may comprise a sensing system comprising an array of sensor units configured to generate a first signal in response to a first analyte, e.g., at least one analyte proportional to O₂ content, and a second signal in response to a second analyte, e.g., at least one analyte proportional to pH value, each sensor unit of the array of sensor units positioned to correspond with a corresponding well on a sample carrier comprising an array of wells; a stage configured to receive the sample carrier; a motion actuator assembly configured to position the stage and/or the sensing system relative to one another on one or more of an x-axis, a z-axis, and a y-axis; a sample temperature control element, e.g., a heating element, configured to control temperature of samples within at least one well (e.g., each well) of the sample carrier to be within a predetermined amount of a sample within another well of the sample carrier; and a signal processing module operatively connected to the sensing system, for example, configured to receive and amplify the first signal and the second signal.

In some embodiments, the sensing system, the stage, the motion actuator assembly, the sample control element, and the signal processing module are contained within a housing. In some embodiments, the housing comprises an opening on a side wall dimensioned to allow passage of the stage and the sample carrier.

In some embodiments, the instrument further comprises the sample carrier. In some embodiments, the sample carrier is disposed on the stage.

In some embodiments, the motion actuator assembly comprises at least one axis actuator assembly, e.g., at least one x-axis actuator assembly.

In some embodiments, the at least one axis actuator assembly, e.g., x-axis actuator assembly is configured to position the stage relative to the sensing system on at least one axis, e.g., the x-axis, e.g., configured to align at least one well (e.g., each well) of the sample carrier disposed on the stage with a corresponding sensor unit on the x-axis. In some embodiments, the at least one axis actuator assembly, e.g., x-axis actuator assembly is configured to position the stage relative to the housing on at least one axis, e.g., the x-axis, e.g., within the housing or exterior to the housing through the opening.

In some embodiments, the motion actuator assembly comprises at least one y-axis actuator assembly. In some embodiments, the at least one axis actuator assembly, e.g., y-axis actuator assembly is configured to position the stage relative to the sensing system on at least one axis, e.g., the y-axis, e.g., configured to align at least one well (e.g., each well) of the sample carrier disposed on the stage with a corresponding sensor unit on the y-axis.

In some embodiments, the motion actuator assembly comprises at least one z-axis actuator assembly. In some embodiments, the at least one axis actuator assembly, e.g., z-axis actuator assembly is configured to position the sensing system relative to the stage on at least one axis, e.g., the z-axis, e.g., configured to position each sensor unit in fluid communication with the corresponding well.

In some embodiments, the sensing system is incorporated in or on a cartridge.

In some embodiments, the analytical instrument further comprises a dispensing system comprising at least one injector configured to dispense at least one target agent into one or more wells of the sample carrier. The instrument may further comprise an injector motion actuator assembly positioned to drive the at least one injector to dispense the at least one target agent across a plurality of wells of the sample carrier.

In some embodiments, the dispensing system may comprise an array of injectors configured to dispense at least one target agent, each injector positioned to correspond with a corresponding well on the sample carrier.

In some embodiments, the array of injectors comprises more than one injector positioned to correspond with at least two wells (e.g., each well) on the sample carrier, e.g., at least 2, 3, 4, 5, 6, 7, 8, 9, or 10 injectors positioned to correspond with at least two wells (e.g., each well) on the sample carrier.

In some embodiments, the analytical instrument further comprises a manifold temperature control element, e.g., a heating element, configured to control temperature of the dispensing system, e.g., target agent, sensing system, and/or cartridge. In some embodiments, the manifold temperature control element and the sample temperature control element are configured to control temperature independently. In some embodiments, the manifold temperature control element is configured to control temperature of the target agent and/or the sensing system and/or the cartridge and the sample temperature control element is configured to control temperature of the samples within the array of wells of the sample carrier to be within 3° C., e.g., 2° C., 1° C., 0.6° C., 0.5° C., 0.4° C., 0.3° C., 0.2° C., or 0.1° C., of the corresponding target agent and/or the corresponding sensor unit.

In some embodiments, the sample temperature control element and/or the manifold temperature control element is configured to control evaporation of the samples within the array of wells to be less than 25%, e.g., less than 20%, 15%, 12%, 10%, 9%, 8%, 7%, 6%, 5%, 4%, 3%, 2%, or 1%.

In some embodiments, the at least one axis actuator assembly, e.g., z-axis actuator assembly is configured to position the dispensing system relative to the stage on at least one axis, e.g., the z-axis, e.g., configured to position each injector in communication, e.g., fluid communication, with the corresponding well, allowing delivery of the target agent to the sample.

In some embodiments, the at least one axis actuator assembly, e.g., z-axis actuator assembly is configured to position the dispensing system relative to the stage on at least one axis, e.g., the z-axis, e.g., configured to position each injector in communication with a corresponding sensor unit of the sensing system.

In some embodiments, the at least one injector or each injector of the array of injectors is configured to dispense the same target agent.

In some embodiments, each injector is configured to independently dispense a selected target agent, e.g., a first injector is configured to dispense a first target agent and a second injector is configured to dispense a second target agent, optionally one or more of a third injector is configured to dispense a third target agent, a fourth injector is configured to dispense a fourth target agent, and a nth injector is configured to dispense a nth target agent. In some embodiments, a plurality of injectors or the array of injectors are configured to independently dispense more than one target agent into at least one well of the sample carrier.

In some embodiments, the sensing system and the dispensing system are incorporated in or on the cartridge.

In some embodiments, the analytical instrument further comprises at least one target agent loaded in the dispensing system.

In some embodiments, the sample temperature control element and/or the manifold temperature control element is formed of a temperature conductive material. In some embodiments, the sample temperature control element is fixed to the stage. In some embodiments, the sample temperature control element is configured to be in close proximity or direct contact with the sample carrier. In some embodiments, the manifold temperature control element is configured to be in close proximity or direct contact with the dispensing system.

In some embodiments, the sample temperature control element is configured to control the temperature of samples within at least one well (e.g., each well) of the sample carrier to be 0° C.-70° C. above ambient temperature, e.g., 8° C.-20° C. above ambient temperature, e.g., 0, 1, 2, 3, 4, 5, 6, 7, 8, 9, 10, 11, 12, 13, 14, 15, 16, 17, 18, 19, 20, 30, 40, 50, 60, or 70° C. above ambient temperature.

In some embodiments, the sample temperature control element is configured to maintain the temperature of samples within at least one well (e.g., each well) of the sample carrier to be within a predetermined range.

In some embodiments, the sample temperature control element is configured to control the temperature of samples within at least one well (e.g., each well) of the sample carrier such that a sensor signal in response to a level, production, or consumption of a target analyte, e.g., the first analyte or the second analyte, does not differ more than a predetermined amount between two identical or substantially identical samples.

In some embodiments, the sample temperature control element is configured to control the temperature of samples within at least one well (e.g., each well) of the sample carrier such that a sensor signal in response to a level, production, or consumption of a target analyte, e.g., the first analyte or the second analyte, does not differ substantially between two identical or substantially identical samples.

In some embodiments, the target analyte is O₂ and the sample temperature control element is configured to control the temperature of samples within at least one well (e.g., each well) such that the sensor signal in response to level, production, or consumption of the target analyte does not differ more than 10%, e.g., does not differ more than 5%, 3%, 1% or 0.1% between two identical or substantially identical signals.

In some embodiments, the target analyte is the analyte proportional to pH value and the sample temperature control element is configured to control the temperature of samples within at least one well (e.g., each well) such that the sensor signal in response to level, production, or consumption of the target analyte does not differ more than 10%, e.g., does not differ more than 5%, 3%, 1%, or 0.1% between two identical or substantially identical samples.

In some embodiments, the sample temperature control element is configured to control the temperature of samples within at least one well (e.g., each well) of the sample carrier to be within 3° C., e.g., within 2° C., 1° C., 0.6° C., 0.5° C., 0.4° C., 0.3° C., 0.2° C., or 0.1° C. of the sample within another well of the sample carrier.

In some embodiments, the manifold temperature control element is configured to control the temperature of sensors to be within 3° C., e.g., within 2° C., 1° C., 0.6° C., 0.5° C., 0.4° C., 0.3° C., 0.2° C., or 0.1° C. of another sensor.

In some embodiments, the sample temperature control element and the manifold temperature control element are configured to control the temperature of samples within at least one well (e.g., each well) of the sample carrier to be within 3° C., e.g., within 2° C., 1° C., 0.6° C., 0.5° C., 0.4° C., 0.3° C., 0.2° C., or 0.1° C. of a corresponding sensor.

In some embodiments, the sample temperature control element is configured to control the temperature of a sample within a first well to be within the predetermined amount of a sample within a second well, wherein the first well is a border well and the second well is an internal well of the sample carrier, wherein the border well is a well which has no other well disposed between the border well and an edge or border of the sample carrier.

In some embodiments, the manifold temperature control element is configured to control the temperature of a sensor corresponding to a first well to be within the predetermined amount of a sensor corresponding to a second well, wherein the first well is a border well and the second well is an internal well of the sample carrier, wherein the border well is a well which has no other well disposed between the border well and an edge or border of the sample carrier.

In some embodiments, the sample temperature control element and/or manifold temperature control element is configured to maintain the temperature of samples within the first well and the second well and/or sensors corresponding with the first well and the second well to be within a predetermined range.

In some embodiments, the sample temperature control element and/or manifold temperature control element is configured to control the temperature of samples (e.g., two identical or substantially identical samples) within the first well and the second well and/or sensors corresponding to the first well and the second well such that a sensor signal in response to level, production, or consumption of a target analyte, e.g., the first analyte or the second analyte, does not differ more than a predetermined amount between each sample, e.g., does not differ substantially between each sample, e.g., when the samples are analyzed under the same or substantially same conditions.

In some embodiments, the sample temperature control element and/or manifold temperature control element is configured to control the temperature of samples (e.g., two identical or substantially identical samples) within the first well and the second well and/or sensor corresponding to the first well and the second well such that a sensor signal in response to level, production, or consumption of a target analyte, e.g., the first analyte or the second analyte, does not differ substantially between each sample, e.g., when the samples are analyzed under the same or substantially the same conditions.

In some embodiments, the target analyte is O₂ and the sample temperature control element and/or manifold temperature control element is configured to control the temperature of samples (e.g., two identical or substantially identical samples) within the first well and the second well and/or sensors corresponding to the first well and the second well, such that the sensor signal in response to level, production, or consumption of the target analyte does not differ more than 10%, e.g., does not differ more than 5%, 3%, 1%, or 0.1% between each sample, e.g., when the samples are analyzed under the same or substantially the same conditions.

In some embodiments, the target analyte is the analyte proportional to pH value and the sample temperature control element and/or manifold temperature control element is configured to control the temperature of samples (e.g., two identical or substantially identical samples) within the first well and the second well and/or sensors corresponding to the first well and the second well such that the sensor signal in response to level, production, or consumption of the target analyte does not differ more than 10%, e.g., does not differ more than 5%, 3%, 1%, or 0.1% between each sample e.g., when the samples are analyzed under the same or substantially the same conditions.

In some embodiments, the sample temperature control element is configured to control the temperature of samples within the first well to be within 3° C., e.g., within 2° C., 1° C., 0.6° C., 0.5° C., 0.4° C., 0.3° C., 0.2° C., or 0.1° C. of the sample within the second well and/or the manifold temperature control element is configured to control the temperature of a sensor correspond with the first well to be within 3° C., e.g., within 2° C., 1° C., 0.6° C., 0.5° C., 0.4° C., 0.3° C., 0.2° C., or 0.1° C. of a sensor corresponding with the second well.

In some embodiments, the first well is a border well and the second well is an internal well of a sample carrier having 1 or more wells, e.g., 6, 8, 12, 24, 36, 48, 64, 72, 96, 192, 384 or more wells. In some embodiments, the sample temperature control element comprises a heating element.

In some embodiments, the sample temperature control element forms a controlled temperature zone which comprises the array of wells of the sample carrier. In some embodiments, the controlled temperature zone does not comprise a headspace of the housing. In some embodiments, a volume of the controlled temperature zone does not exceed a volume of the sample carrier by more than 1, 2, 3, 4, 5, 6, 7, 8, 9, or 10-fold. In some embodiments, a volume of the controlled temperature zone does not exceed 10%, e.g., 9%, 8%, 7%, 6%, 5%, 4%, 3%, 2%, or 1%, of a volume of the housing. In some embodiments, a temperature of components outside the controlled temperature zone is not substantially altered, e.g., increased or decreased, by activation of the sample temperature control element.

In some embodiments, the sample temperature control element is configured to bring the temperature of samples within at least one well (e.g., each well) of the sample carrier to be within a predetermined range of a target temperature within about 5 hours, 3 hours, 1 hour, 45 minutes, 30 minutes, 15 minutes, 10 minutes, 5 minutes, 3 minutes, or 1 minute of activation of the sample temperature control element and/or introduction of the sample carrier into the controlled temperature zone.

In some embodiments, the sample temperature control element and/or the manifold temperature control element is configured to control temperature to control, e.g., reduce, limit, or inhibit, diffusion of gases in the controlled temperature zone. In some embodiments, the sample temperature control element and/or the manifold temperature control element is configured to control temperature to reduce, limit, or inhibit, the diffusion of gases in the controlled temperature zone such that a composition of gases in the controlled temperature zone does not vary significantly e.g., does not vary more than 1%, 2%, 4%, 6%, 8%, 10%, 12%, 14%, 16%, 18%, or 20%.

In some embodiments, the signal processing module is capable of operating at increased relative humidity (RH), e.g., at least 75% RH, 85% RH, 95% RH, or 99% RH.

In some embodiments, the signal processing module is configured to receive and amplify the first signal and the second signal simultaneously.

In some embodiments, the signal processing module is configured to receive and amplify the first signal and the second signal individually, e.g., sequentially.

In some embodiments, the signal processing module is configured to detect one or more of the first signal and the second signal using time-based detection, e.g., rate of decay, phase shift, or anisotropy detection.

In some embodiments, the signal processing module is configured to detect one or more of the first signal and the second signal using intensity-based detection, optionally including a ratiometric measurement.

In some embodiments, the signal processing module comprises a printed circuit assembly formed of an insulating material having a high dielectric constant.

In some embodiments, the signal processing module comprises a printed circuit assembly having a transimpedance amplifier including grounded guard traces.

In some embodiments, the signal processing module comprises a printed circuit assembly formed of surface mount components, e.g., substantially free of secondary hand soldered high gain components.

In some embodiments, the signal processing module comprises a printed circuit assembly comprising a thermally conductive excitation source, optionally wherein the thermally conductive excitation source is in thermal communication, e.g., thermal contact, with a thermal sink. In some embodiments, the sensing system does not include a reference signal detector.

In some embodiments, the signal processing module is configured to operate with reduced parasitic current, e.g., reduced interference, dark currents, or noise, associated with the detection and/or amplification of at least one of the first signal and the second signal.

In some embodiments, the sensing system is constructed and arranged to form a measurement chamber between a sample-facing surface of each sensor unit and a sensor-facing surface of at least one well (e.g., each well) when the motion actuator assembly is deployed to position each sensor unit in fluid communication with the sample within the corresponding well, wherein evaporation of the sample, flow of the sample, or diffusion of a component, e.g., analyte, of the sample out of the measurement chamber is impaired. In some embodiments, the sample temperature control element is configured to control temperature of samples within each measurement chamber.

In some embodiments, the sensing system comprises one or more optical sensors, e.g., photoluminescence sensors.

In some embodiments, the sensing system comprises one or more electrochemical sensors.

In some embodiments, the sensing system is configured to generate a signal in response to rate of change of an analyte proportional to O₂ content in the sample, e.g., generates a signal proportional to oxygen consumption rate (OCR) of the sample.

In some embodiments, the sensing system is configured to generate a signal in response to rate of change of an analyte proportional to pH value of the sample, e.g., generates a signal proportional to extracellular acidification rate (ECAR) and/or proton efflux rate (PER) of the sample.

In some embodiments, the sensing system is configured to generate a signal in response to one or more electrochemical property of the sample, e.g., impedance.

In some embodiments, the signal processing module is operatively connected to a computing network or computer device programmed to calculate one or more of mitochondrial respiration, glycolysis, adenosine triphosphate (ATP) production rate, and mitochondrial toxicity (mitotox) index value of the sample responsive to one or more of the first signal and the second signal. In some embodiments, the signal processing module is operatively connected to a cloud-based computing network. In some embodiments, the signal processing module is operatively connected to a data storage module storing historical values for the first signal and the second signal. In some embodiments, the data storage module is a local memory storage device. In some embodiments, the data storage module is a cloud-based memory storage device.

In some embodiments, the array of sensor units comprises Xₛ₁ sensor units, and Xₛ₁ is equal to or greater than 1, 6, 8, 12, 24, 36, 48, 64, 72, 96, 192, or 384.

In some embodiments, the array of injectors comprises Xₛ₂ injectors, and Xₛ₂ is equal to or greater than 1, 6, 8, 12, 24, 36, 48, 64, 72, 96, 192, 384, 768, or 1536.

In some embodiments, the instrument has a ratio of sensor units Xₛ₁ to injectors Xₛ₂ of 1:1 to 1:384, e.g., 1:1, 1:2, 1:3, 1:4, 1:8, 1:12, 1:24, 1:36, 1:48, 1:64, 1:72, 1:96, 1:192, or 1:384. In some embodiments, the instrument has a ratio of injectors Xₛ₂ to sensor units Xₛ₁ of 1:1 to 1:384, e.g., 1:1, 1:2, 1:3, 1:4, 1:8, 1:12, 1:24, 1:36, 1:48, 1:64, 1:72, 1:96, 1:192, or 1:384.

In some embodiments, each sensor unit of the array of sensor units is configured to generate one or more of the first signal and the second signal independently.

In some embodiments, each sensor unit of the array of sensor units is configured to generate the first signal and the second signal concurrently.

In some embodiments, the analytical instrument further comprises a light source, e.g., a fluorescent light, light emitting diode (LED), or laser, configured to excite a sensor of the sensor unit to generate one or more of the first signal and the second signal. In some embodiments, the light source is configured to produce a reference signal, wherein fluctuations in intensity from the light source are corrected proportionally to drift by monitoring the reference signal produced by the light source. In some embodiments, the light source is positioned on a thermally conductive printed circuit assembly configured to minimize drift from the light source, optionally wherein the thermally conductive printed circuit assembly is formed of a material configured to minimize drift generated by heat-induced fluctuations from the light source by at least 20%, e.g., at least 15%, 10%, 5%, or 1%.

In some embodiments, the analytical instrument further comprises an electric motor configured to actuate the motion actuator assembly, e.g., one or more of the x-axis actuator assembly, the z-axis actuator assembly, and the y-axis actuator assembly.

In some embodiments, the analytical instrument further comprises a stall sensing module programmed to generate a notification signal, and optionally pause a protocol, e.g., halt motor movement, if a predetermined protocol step is not completed within a predetermined time interval.

In some embodiments, the analytical instrument further comprises a proximity sensor configured to generate a notification signal, and optionally pause a protocol, if a component is positioned within a predetermined distance from another component, e.g., a sensor unit within a predetermined distance from a corresponding well of the sample carrier.

In some embodiments, the analytical instrument further comprises a proximity sensor configured to generate a notification signal, and optionally pause a protocol, if the opening on the side wall of the housing is ajar and/or external light is detected within the housing.

In some embodiments, the analytical instrument has an OCR detection range of 2000 pmol/min to 0.01 pmol/min, e.g., 700 pmol/min to 0.01 pmol/min, e.g., 50 pmol/min to 0.01 pmol/min.

In some embodiments, the analytical instrument has a lower OCR detection limit of less than 50 pmol/min, e.g., less than 40 pmol/min, 30 pmol/min, 20 pmol/min, 10 pmol/min, 5 pmol/min, 3 pmol/min, 1 pmol/min, 0.1 pmol/min, or 0.01 pmol/min.

In some embodiments, the analytical instrument further comprises an optical module positioned to image or scan one or more samples within the array of wells of the sample carrier. In some embodiments, the optical module is operatively connected to the computer, optionally wherein the computer is configured to display and/or record the image or scan of the samples in real time.

In some embodiments, the analytical instrument further comprises a transfer module formed of a multiplexed fiber optic material configured to transfer optical signals from the array of sensor units to the signal processing module. In some embodiments, the transfer module is configured to transfer one or more of excitation, reference, and emission optical signals. In some embodiments, the transfer module is configured to directly interface with one or more sensor units. In some embodiments, the sensing system comprises a homogenized fiber optic wave guide optically connected to the transfer module, optionally wherein the homogenized fiber optic wave guide is configured to uniformly distribute light onto one or more sensor units.

In some embodiments, the instrument further comprises an environmental control module configured to control an environment of samples within at least one well (e.g., each well) of the sample carrier, e.g., configured to control environmental gas and/or relative humidity (RH). In some embodiments, the environmental control module is configured to control one or more of N₂, O₂, and CO₂ concentration of the gas surrounding the samples. In some embodiments, the environmental control module comprises a source of a gas, e.g., one or more of N₂, O₂, and CO₂, fluidly connected to the sample carrier. In some embodiments, the environmental control module forms a controlled environment zone which comprises the array of wells of the sample carrier. In some embodiments, the controlled environment zone is formed in a sealed container, e.g., hermetically sealed container.

In some embodiments, the instrument is configured for use within a gas-controlled environment.

In accordance with another aspect, there is provided a method of using the analytical instrument comprising loading a sample carrier comprising one or more cell samples, each sample disposed within a corresponding well of the sample carrier into the analytical instrument.

In some embodiments, the cell samples comprise live cells. In some embodiments, loading the sample carrier comprises fixing the sample carrier onto the stage.

In accordance with yet another aspect, there is provided a method of analyzing a cell sample. The method may comprise providing an analytical instrument; loading a sample carrier comprising one or more cell samples, each sample disposed within a corresponding well of the sample carrier into the analytical instrument; obtaining a first plurality of values from signals in response to the first analyte, e.g., at least one analyte proportional to O₂ content, each signal generated by a corresponding sensor unit of the sensing system; optionally, obtaining a second plurality of values from signals in response to the second analyte, e.g., at least one analyte proportional to pH value, each signal generated by the corresponding sensor unit of the sensing system; processing the first plurality of values; and optionally, processing the second plurality of values.

In some embodiments, the method further comprises controlling the temperature of samples within at least one well (e.g., each well) of the sample carrier to be within the predetermined amount of the sample within another well of the sample carrier.

In some embodiments, the sample carrier is loaded into the controlled temperature zone and/or wherein controlling the temperature of samples includes forming the controlled temperature zone.

In some embodiments, the method further comprises controlling temperature of the sensing system.

In some embodiments, the method further comprises dispensing a target agent into each sample within the array of wells of the sample carrier. In some embodiments, the method further comprises loading the target agent into the dispensing system of the analytical instrument. In some embodiments, the method further comprises controlling temperature of the target agent.

In some embodiments, the same sample is present within at least one well (e.g., each well) of the array of wells of the sample carrier.

In some embodiments, a first sample is present in a first well of the array of wells of the sample carrier and a second sample is present in a second well of the array of wells of the sample carrier. In some embodiments, the first sample is a test sample and the second sample is a control.

In some embodiments, the sample comprises live cells.

In some embodiments, the sample comprises one or more of loose cells, cell constructs, loose tissue, tissue constructs, organelles, enzymes, cell products or byproducts, and conditioned medium.

In some embodiments, the sample comprises mammalian cells or tissue. In some embodiments, the sample comprises non-mammalian cells or tissue. In some embodiments, the sample comprises single-celled organisms, e.g., microorganisms. In some embodiments, the sample comprises whole animal model tissues, e.g., zebrafish, C. elegans, drosophila. In some embodiments, the sample comprises whole plant model tissues or plant model cells.

In some embodiments, the first analyte is proportional to O₂ content.

In some embodiments, the second analyte is proportional to pH value.

In some embodiments, the first value and the second value are obtained independently.

In some embodiments, the first value and the second value are obtained concurrently.

In some embodiments, the method further comprises obtaining an image or scan of the samples during or after the analysis.

In some embodiments, the method further comprises measuring one or more electrochemical property, e.g., impedance, of the samples during or after the analysis.

In some embodiments, the method further comprises obtaining or calculating a mitochondrial toxicity (mitotox) index value of the samples during or after the analysis.

In some embodiments, the method further comprises controlling the environment of samples within at least one well (e.g., each well) of the sample carrier, e.g., controlling environmental gas and/or relative humidity (RH). In some embodiments, controlling environment includes controlling one or more of N₂, O₂, and CO₂ concentration of the gas surrounding the samples.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a front perspective view of an apparatus for analyzing live cells, according to one embodiment;

FIG. 2 is a back perspective view of an apparatus for analyzing live cells, according to one embodiment;

FIG. 3 is a side view of an apparatus for analyzing live cells, according to one embodiment;

FIG. 4 is a top view of an apparatus for analyzing live cells, according to one embodiment;

FIG. 5 is a side view of select components of an apparatus for analyzing live cells, according to one embodiment;

FIG. 6 is a schematic drawing of a system for analyzing live cells, according to one embodiment;

FIG. 7 is a schematic drawing of a system for analyzing live cells, according to one embodiment;

FIG. 8 is a schematic drawing of select components of a system for analyzing live cells, according to one embodiment;

FIG. 9 is a schematic drawing of select components of an apparatus for analyzing live cells, according to one embodiment;

FIGS. 10A-10D are graphs showing oxygen consumption rate (OCR) or extracellular acidification rate (ECAR) of live cells analyzed by the methods disclosed herein, according to one embodiment, in particular, FIGS. 10A-10B are OCR readings from an analytical instrument as disclosed herein and a comparative analytical instrument, respectively, showing an improved, stable performance at 0-15 minutes from the analytical instrument as compared to the comparative instrument; and FIGS. 10C-10D are ECAR readings from an analytical instrument as disclosed herein and a comparative analytical instrument, respectively;

FIGS. 11A-11B are graphs showing oxygen consumption rate (OCR) of live cells analyzed by the methods disclosed herein, according to one embodiment, in particular FIGS. 11A-11B are OCR readings from an analytical instrument as disclosed herein and a comparative analytical instrument, respectively, demonstrating results with significantly lower variation, particularly with metformin treated cells (lower line);

FIG. 12 is a table showing average evaporation of a substance (e.g., media) contained in the sample carrier analyzed by the methods disclosed herein, according to one embodiment;

FIGS. 13A-13B are comparative illustrations of a spheroid;

FIG. 14 is a block diagram illustrating a multi-detection system according to an embodiment;

FIG. 15 is a block diagram illustrating a multi-detection system according to an embodiment;

FIG. 16 is a block diagram illustrating a multi-detection system according to an embodiment;

FIG. 17 is a block diagram illustrating a multi-detection system according to an embodiment;

FIG. 18 is a diagram illustrating a spinning disk according to an embodiment;

FIG. 19 is a diagram illustrating a confocal disk imaging module according to an embodiment;

FIG. 20 illustrates a disk changing mechanism and a disk focus mechanism according to an embodiment;

FIG. 21 is a diagram of a non-imaging analyzing subsystem according to an embodiment;

FIG. 22 is a diagram illustrating an injection subsystem according to an embodiment;

FIG. 23 is a diagram illustrating a multi-detection system according to an embodiment;

FIG. 24A is a perspective view illustrating an environmental control subsystem according to an embodiment;

FIG. 24B is a rear view illustrating the environmental control subsystem according to the embodiment;

FIG. 24C is a front view illustrating the environmental control subsystem according to the embodiment;

FIG. 25 is a functional block diagram that illustrates the control of modalities of the instrument according to an embodiment; and

FIG. 26 is a flowchart of a control method of a multi-detection system according to an example embodiment;

FIG. 27A is a first diagram illustrating a liquid immersion objective according to an embodiment;

FIG. 27B is a second diagram illustrating the liquid immersion objective according to the embodiment;

FIG. 28 is a diagram illustrating a fluid pump system according to an embodiment;

FIG. 29 is a diagram illustrating an objective coupling according to an embodiment;

FIG. 30A is a perspective view illustrating a liquid immersion objective according to a first embodiment;

FIG. 30B is a top view illustrating the liquid immersion objective according to the first embodiment;

FIG. 30C is a first cross-sectional view, taken along line A-A in FIG. 30B, illustrating the liquid immersion objective according to the first embodiment in a state in which a liquid bulb is provided;

FIG. 30D is a second cross-sectional view, taken along line A-A in FIG. 30B, illustrating the liquid immersion objective according to the first embodiment, over which a sample carrier (e.g., a microplate) is provided;

FIG. 31A is a top view illustrating a liquid immersion objective according to a second embodiment;

FIG. 31B is a first cross-sectional view, taken along line B-B in FIG. 31A, illustrating the liquid immersion objective according to the second embodiment, in a state in which a liquid bulb is provided;

FIG. 31C is a second cross-sectional view, taken along line B-B in FIG. 31A, illustrating the liquid immersion objective according to the second embodiment, over which a sample carrier (e.g., a microplate) is provided;

FIG. 32A is a top view illustrating a liquid immersion objective according to a third embodiment;

FIG. 32B is a first cross-sectional view, taken along line C-C in FIG. 32A, illustrating the liquid immersion objective according to the third embodiment, in a state in which a liquid bulb is provided;

FIG. 32C is a second cross-sectional view, taken along line C-C in FIG. 32A, illustrating the liquid immersion objective according to the third embodiment, over which a sample carrier (e.g., a microplate) is provided;

FIG. 33A is a top view illustrating a liquid immersion objective according to a fourth embodiment;

FIG. 33B is a first cross-sectional view, taken along line D-D in FIG. 33A, illustrating the liquid immersion objective according to the fourth embodiment, in a state in which a liquid bulb is provided;

FIG. 33C is a second cross-sectional view, taken along line D-D in FIG. 33A, illustrating the liquid immersion objective according to the fourth embodiment, over which a sample carrier (e.g., a microplate) is provided;

FIG. 34A is a first diagram of a multi-detection system in a laser point scanning confocal modality according to an embodiment;

FIG. 34B is a second diagram of the multi-detection system in a wide field or spinning disk confocal modality according to the embodiment;

FIG. 35 is a diagram of an example user interface according to an embodiment;

FIG. 36 is a schematic diagram showing a cross-sectional view of a transfer module, according to one embodiment;

FIG. 37 is a drawing of a side view of a transfer module, according to one embodiment;

FIG. 38 includes graphs showing precision of measurements taken with an instrument having a thermally conductive excitation source, according to one embodiment;

FIGS. 39A-39C show Mito toxicity results in a negative MTI value;

FIGS. 40A-40C show Mito toxicity results in a positive MTI value;

FIG. 41 includes exemplary MTI detection graphs;

FIG. 42 includes graphs showing kinetic dose response OCR data for 3 test compounds; and

FIG. 43 is a graph showing Z′ values achieved using MitoTox assay and MTI-based analysis, according to one embodiment;

FIG. 44 is a schematic drawing of a 96-well plate wherein shaded circles show which wells were plated with each cell density for use in measuring the oxygen consumption rate of live cells analyzed by the methods disclosed herein, according to one embodiment;

FIGS. 45A-45C are graphs showing the oxygen consumption rate (OCR) of live cells analyzed by the methods disclosed herein, according to one embodiment, in particular, FIGS. 45A-45C are three experimental replicates of OCR readings from an analytical instrument as disclosed herein (left) and a comparative analytical instrument (right), demonstrating results with significantly lower variation, particularly at lower OCR rates when cells were plated at lower densities or after cells were treated with respiration inhibitors (Oligomycin or Rotenone+Antimycin A), from the analytical instrument as compared to the comparative instrument;

FIGS. 46A-46B are graphs of the same data showing basal oxygen consumption rate (OCR) of live cells analyzed by the methods disclosed herein, according to one embodiment, in particular, FIGS. 46A-46B are basal OCR readings from an analytical instrument as disclosed herein (left) and a comparative analytical instrument (right), demonstrating results with significantly lower variation, particularly at lower OCR rates when cells were plated at lower densities, from the analytical instrument as compared to the comparative instrument;

FIG. 47 is a graph showing the standard deviations of basal oxygen consumption rates (OCR) of live cells analyzed by the methods disclosed herein, according to one embodiment, across three experimental replicates, in particular, FIG. 46 is the standard deviations of basal OCR readings from an analytical instrument as disclosed herein (gray) and a comparative analytical instrument (black) demonstrating results with significantly lower variation from the analytical instrument as compared to the comparative instrument.

### DETAILED DESCRIPTION

Bioenergetic capacity drives biological processes of cells, with cellular metabolism being a central indicator of biological function and cell health. The devices and methods disclosed herein may be used to measure metabolic pathways of cells with high throughput screening techniques. Thus, the devices and methods disclosed herein may be employed to determine and/or quantify key indicators of healthy cell function, predictions of cellular performance in in vitro disease models and drug/compound/substance discovery, through modulation of metabolic targets, signaling, and substrates, with the aim of better understanding the disease state, allowing insight into appropriate therapies to change the disease state, a healthy phenotype, and/or to optimize and enhance cell performance.

The devices and methods disclosed herein may be employed to measure two main metabolic pathways, mitochondrial respiration and glycolysis, for live cells in real time, to provide functional kinetic measurements of cellular bioenergetic capacity.

The devices and methods disclosed herein may be provided to facilitate testing of disease models and critical cell processes including activation, proliferation, differentiation, cell death, cellular homeostasis, and/or disease progression; therapeutic discovery by revealing and validating potential therapeutic drug/compound/substance targets; and optimize the engineering and manufacturing of cell therapies.

In one embodiment, the mitochondrial respiration, glycolic activity and/or metabolic poise is a temporal measurement of a cell's activity independent of the media/buffer surrounding the cell. Creating a microchamber allows sensitive measurements of cellular activity to be detected. A change in the cell mitochondrial respiration and/or glycolytic activity results in micro-changes of O₂, CO₂, lactate in the immediate environment surrounding the cell in real time, this change in the immediate environment is detected by the device by OCR, ECAR, and/or PER measurements.

In another embodiment, the mitochondrial respiration and/or glycolytic activity is a temporal measurement of a cellular activity influenced by the media/buffer surrounding the cell by the addition of gases, therapeutic drug targets or agents that impact the cell activity such as ATP synthase inhibitor, mitochondrial uncoupling agent, or ETC inhibitor to the media that influence the cell.

In particular, the devices and methods disclosed herein may be employed to measure oxygen consumption rate (OCR), extracellular acidification rate (ECAR), proton efflux rate (PER), adenosine triphosphate (ATP) production rate, and other parameters of a plurality of cell samples in a sample carrier (e.g., a multi-well plate). OCR and ECAR or PER may be used to determine mitochondrial respiration and glycolysis as well as ATP production rate. The measurements obtainable by the devices and methods disclosed herein may provide a comprehensive view of cellular metabolic function in cultured cell samples and ex vivo samples.

It should be noted that cell samples as described herein may include loose cells, cell constructs, loose tissue, and tissue construct samples. Cell samples may be or include organelles, enzymes, cell products or byproducts, and/or conditioned medium. Parameters for each cell sample (each well) may be independently and selectively measured. In particular embodiments, live cell samples may be tested, for example, without a significant reduction in cell viability. The devices and methods described herein may provide a lower dissolved oxygen or OCR detection limit, greater consistency in accurateness, improved temperature control, and improved automation over conventional devices and methods.

Conventional systems are susceptible to humidity and contamination caused by factors such as laboratory environment, storage, and manufacturing processes, tend to experience motion errors over time, including inconsistencies in movement/buildup of debris, and are susceptible to evaporation, edge well temperature gradients, and long warm up times caused by an environmental heating approach. The devices and methods disclosed herein contain components that overcome these drawbacks of the conventional systems, resulting in improved measurement performance which surprisingly provides a lower detection limit of O₂ and improved precision of measurements.

The combination of hardware and analysis software provided in the instruments disclosed herein allows real-time monitoring of live cells in areas such as immunology and disease using rare, ex vivo, and genetically engineered cells to build better disease models. The enhancements disclosed herein improve measurement performance. These enhancements generally make it easier to identify novel drug/compound/substance targets, validate target effect on cellular function, optimize disease models, and determine drug/compound/substance safety and antitumor potential of T cell therapies going from research laboratories to biopharma therapeutic development and toxicity programs.

The instrument disclosed herein is capable of delivering better precision at a low oxygen consumption rate (OCR), allowing analysts to confidently interrogate more immune cell types, as well as cell types that are bioenergetically compromised.

The devices and methods disclosed herein provide the ability to analyze live cells in an extended temperature range. For instance, the temperature control element and controlled temperature zone that is smaller than the headspace of the housing contribute to improvements over previous devices.

The devices and methods disclosed herein provide more uniformity in heating the temperature control element which may improve cell biology at a consistent temperature and sensing with the instrument sensors, reducing systemic edge effects.

The devices and methods disclosed herein can provide temperature control at a faster start up time than previous devices.

The devices and methods disclosed herein include electronic optics boards capable of performing at humidity levels as high as 95%. Performance of previous instruments is often less than optimal at 70%-80% humidity. Thus, the instrument may be transported, stored, or used in territorial regions of high humidity

The devices and methods disclosed herein provide improved performance and detection at the lower levels of OCR that previously appeared as noise, which allows analysis of damaged or compromised immune cells, thereby widening the different types of cells that can be analyzed by the instrument.

In some embodiments, the devices describe herein measure the OCR and ECAR/PER of live cells in a 96-well format. Without wishing to be bound by theory, it is believed that in some embodiments, the devices described herein feature better OCR precision at low rates, verified instrument performance and repeatability specifications, optimized temperature control, and are automation allowed.

### Definitions

Unless defined otherwise, all technical and scientific terms used herein have the same meaning as commonly understood by one of ordinary skill in the art to which the invention pertains.

As used herein, the articles “a” and “an” refer to one or to more than one (e.g., to at least one) of the grammatical object of the article.

“About” and “approximately” as the term used herein shall generally mean an acceptable degree of error for the quantity measured given the nature or precision of the measurements. Exemplary degrees of error are within 20 percent (%), typically, within 10%, and more typically, within 5% of a given value or range of values.

“Acquire” or “acquiring” as the term used herein refers to obtaining possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent. In an embodiment, directly acquiring encompasses a direct measurement. In an embodiment, indirectly acquiring encompasses an inference.

“Acquiring a sample” as the term used herein refers to obtaining possession of a sample, e.g., a sample described herein, by “directly acquiring” or “indirectly acquiring” the sample. “Directly acquiring a sample” means performing a process (e.g., performing a physical method such as a surgery or extraction) to obtain the sample. “Indirectly acquiring a sample” refers to receiving the sample from another party or source (e.g., a third-party laboratory that directly acquired the sample). Directly acquiring a sample includes performing a process that includes a physical change in a physical substance, e.g., a starting material, such as a tissue, e.g., a tissue in a human patient or a tissue that has was previously isolated from a patient. Exemplary changes include making a physical entity from a starting material; dissecting or scraping a tissue; separating or purifying a substance; combining two or more separate entities into a mixture; or performing a chemical reaction that includes breaking or forming a covalent or non-covalent bond.

“Ambient temperature” as the term used herein refers to the air temperature of the environment or immediate surroundings Ambient temperature may also be referred to as baseline temperature or the temperature of the device or object before temperature control is activated. In certain embodiments, ambient temperature may be between 1° C. and 60° C. In certain embodiments, ambient temperature may be between 18° C. to 25° C. In certain embodiments, ambient temperature may be between 1° C. to 5° C. In certain embodiments, ambient temperature may be between 32° C. to 60° C.

“Basal mitochondrial ATP production rate” as the term used herein refers to the rate of ATP production by mitochondria in a cell sample before the cell sample is contacted with an ATP synthase inhibitor, a mitochondrial uncoupling agent, and an electron transport chain (ETC) inhibitor to form a reaction mixture. In an embodiment, the basal mitochondrial ATP production rate is calculated by subtracting the minimum oxygen consumption rate (oligo OCR) to a measurement (e.g., the last measurement, or an average of a number of measurements) of oxygen consumption rate before the first contacting of the cell sample with any of the ATP synthase inhibitor, mitochondrial uncoupling agent, or ETC inhibitor (basal OCR) and multiplying by a constant between 2.45 and 2.86 (called P/O ratio)*2 (to convert oxygen atoms to oxygen molecules). In an embodiment, the constant is 2.75.

“Bioenergetic capacity” as the term used herein refers to the level of increase in glycolytic and/or mitochondrial activity that a cell can affect, utilize, and/or induce. In an embodiment, the bioenergetic capacity is determined in response to increased energy demand and/or in response to inhibition/perturbation of energy-generation. In an embodiment, the bioenergetic capacity comprises a value for oxygen consumption (e.g., an oxygen consumption rate (OCR)) and a value for proton efflux (e.g., a proton efflux rate (PER)). In an embodiment, the value for oxygen consumption (e.g., OCR) is in response to mitochondrial uncoupling. In an embodiment, the value for proton efflux (e.g., PER) in in response to ATPase inhibition. In an embodiment the PER is glycolytic PER (glycoPER), which mathematically removes the contribution of CO₂.

“Bioenergetic poise” as the term used herein refers to the balance between aerobic and glycolytic energy production. In an embodiment, the bioenergetic poise describes the proportion of ATP generated by glycolysis of oxidative phosphorylation. In an embodiment, the bioenergetic poise comprises a relationship, e.g., a ratio, between ATP made by mitochondria and ATP made by glycolysis, between ATP made by mitochondria and total ATP production, between ATP made by glycolysis and total ATP production, or any combination thereof.

“Bioenergetic work” as the term used herein refers to the amount of ATP being generated by a cell.

“Cell sample” as the term used herein refers to a sample that comprises a cell or a cell product or byproduct. In an embodiment, the cell sample comprises a plurality of cells. In an embodiment, the cell is disposed in a medium. The cell sample may be or comprise one or more of cells, tissue, cell or tissue constructs, organelles, enzymes, and/or conditioned medium.

“Cellular metabolic function” as the term used herein refers to a living organism's ability to perform chemical reactions necessary to maintain life. In embodiments, cellular metabolic function of a cell sample may be monitored by measuring OCR and ECAR.

“Extracellular acidification rate (ECAR)” as the term used herein refers to a measurement of proton extrusion in the extracellular medium over time. ECAR may be reported as rate of change of pH units, e.g., milli-pH/minute (mpH/min), over assay run time.

“Glycolysis” or “glycolytic activity” as the term used herein refers to the cellular metabolic function of converting glucose is into lactate.

“Mitochondrial respiration” as the term used herein refers to the metabolic reactions and processes requiring oxygen that take place in mitochondria to convert the energy stored in macronutrients into ATP.

“Mitochondrial toxicity index” (also referred to as “mitotox index” or “MTI”) as the term used herein refers to index values derived from OCR measurements. MTI is a parameter that provides information for both the type and magnitude of mitochondrial toxicity. Positive MTI values (typically between 0 and 1) identify mitochondrial toxicity due to uncoupling, and conversely, negative MTI values (typically between 0 and −1) identify mitochondrial toxicity due to inhibition.

“Or” is used herein to mean, and is used interchangeably with, the term “and/or”, unless context clearly indicates otherwise. The use of the term “and/or” in some places herein does not mean that uses of the term “or” are not interchangeable with the term “and/or” unless the context clearly indicates otherwise.

“Oxygen consumption rate (OCR)” as the term used herein refers to a quantitative measurement of oxygen consumption by the sample over time. Accordingly, OCR may provide a measure of cellular and mitochondrial respiration over time. OCR values may be reported in rate of change of O₂ content, e.g., picomole/minute (pmol/min) over assay run time.

In one embodiment OCR includes those scenarios in which oxygen consumption is not determined in a completely sealed system, e.g., a system allows oxygen back diffusion or substantial oxygen back diffusion to the sample, or where oxygen consumption is oxygen depletion in the sample corrected for oxygen back diffusion to the sample, or oxygen consumption is oxygen depletion without being corrected for oxygen back diffusion to the sample, or the oxygen consumption is determined in a sealed system, e.g., a system that does not allow oxygen back diffusion or substantial oxygen back diffusion to the sample, or oxygen consumption equals, or substantially equals, to oxygen depletion in the sample.

In one embodiment, oxygen consumption is determined directly or indirectly, e.g., inferred from a measured oxygen gradient, e.g., within a test well, or across a capillary, or by measuring oxygen at a preselected timepoint.

In one embodiment, oxygen consumption is reported in units other than rate of change of O₂ content, e.g. sensor response per unit time (such as, microseconds/min, relative fluorescence units/min).

“Primary cell” as the term used herein refers to a cell isolated or harvested directly from a subject, organ, or tissue. For example, primary cells can be isolated from blood obtained from a living subject. Primary cells can be isolated or harvested using enzymatic or mechanical methods. Once isolated or harvested, primary cells can be cultured in media containing essential nutrients and growth factors to support proliferation. Primary cells can be suspension cells that do not require attachment for growth (e.g., anchorage-independent cells) or adherent cells that require attachment for growth (e.g., anchorage-dependent cells).

“Proton efflux rate (PER)” as the term used herein refers to a quantitative measure of extracellular acidification that accounts for media buffering capacity and plate geometry. PER values may be reported in rate of change of H⁺, e.g., picomole/minute (pmol/min) over assay run time. H⁺ is a quantifiable analyte proportional to pH value.

“Sample” as the term used herein refers to a biological sample obtained or derived from a source of interest. In an embodiment, the source of interest comprises an organism, such as an animal or human. The source of the sample can be blood or a blood constituent; a bodily fluid; a solid tissue as from a fresh, frozen and/or preserved organ, tissue, biopsy, resection, smear, or aspirate; or cells from any time in gestation or development of a subject. In an embodiment, the source of the sample is blood or a blood constituent. In an embodiment, the sample is a primary sample, e.g., obtained directly from a source of interest by any appropriate means. In an embodiment, the sample is a preparation that is obtained by processing (e.g., by removing one or more components of and/or by adding one or more agents to) a primary sample.

“Sample carrier” as the term used herein refers to a substrate in which a sample can be carried. In an embodiment, the sample carrier may contain one or more wells. Exemplary sample carriers include, but are not limited to, a microplate, a microtiter plate, a multi-well plate, a single-well plate, a micro-well plate, a microfluidic-chip, microfluidic device, a dish, a slide, a flask, and a tube. The samples carrier can be used to hold various types of samples, including, but not limited to, cells, tissues, small organisms, animal models, multicellular structures, and 3D samples. As used herein, at least one well of the sample carrier can mean at least 1, 2, 3, 4, 5, 6, 12, 18, 24, 30, 36, 42, 48, 54, 60, 66, 72, 78, 84, 90, 96, 192, 288, 384, or 1536 wells, or any number of wells in between or more. As used herein, at least two wells of the sample carrier can mean at least 2, 3, 4, 5, 6, 12, 18, 24, 30, 36, 42, 48, 54, 60, 66, 72, 78, 84, 90, 96, 192, 288, 384, or 1536 wells, or any number of wells in between or more.

Bioenergy Measurement

The two major pathways to produce energy, mitochondrial respiration, and glycolysis, involve cellular consumption of oxygen and efflux of protons, respectively. The devices and methods disclosed herein include sensors, e.g., label-free sensors, to detect extracellular changes in analytes and measure rates of cellular respiration, glycolysis, and ATP production. The apparatus described herein may be employed for determining extracellular, intracellular, and pericellular analytes.

In accordance with certain embodiments, disclosed herein is an analytical instrument, also referred to as an apparatus herein. The apparatus may include a stage adapted to support a sample carrier (e.g., a multi-well plate), also referred to as a sample carrier or a sample carrier cartridge herein. The apparatus may include a sensor adapted to sense a cell constituent associated with the cell sample in a well of the sample carrier (e.g., the multi-well plate). The apparatus may include a dispensing system adapted to introduce fluids into the well. The apparatus may include a plunger adapted to receive a barrier to create a reduced volume of media within the well including at least a portion of the cells, the barrier adapted for insertion into the well by relative movement of the stage and the plunger.

In particular, the apparatus may include a plurality of sensors, each sensor adapted to sense a cell constituent of a corresponding well of the sample carrier (e.g., the multi-well plate). Thus, the apparatus may include an array of sensors. The sensors may independently and selectively sense the cell constituent of at least one well (e.g., each well). The dispensing system may include one or more injectors. The dispensing system may be configured to introduce fluids or agents independently and selectively into at least one well (e.g., each well). The plunger may be adapted to independently and selectively be inserted into at least one well (e.g., each well).

The apparatus may include a motion actuator assembly, also referred to as an elevator mechanism herein, constructed and arranged to position or orient one or more components along at least one coordinate axis. The motion actuator assembly may include one or more high torque motors configured to drive system components.

The motion actuator assembly may include at least one axis actuator assembly. In some embodiments, the motion actuator assembly may include at least one x-axis actuator assembly configured to position the stage relative to the sensor. The x-axis actuator assembly may additionally or alternatively be configured to position the sensor relative to the stage. The x-axis actuator assembly may additionally or alternatively be configured to position the stage relative to the housing. The motion actuator assembly may include at least one z-axis actuator assembly configured to position the sensor and/or dispensing system relative to the stage. The z-axis actuator assembly may additionally or alternatively be configured to position the stage relative to the sensor and/or dispensing system. The motion actuator assembly may include at least one y-axis actuator assembly configured to position the stage relative to the sensor. The y-axis actuator assembly may additionally or alternatively be configured to position the sensor relative to the stage.

In use, the motion actuator assembly may be configured to align or substantially align the array of sensor units and/or injectors with corresponding wells of the sample carrier (e.g., the multi-well plate) positioned on the stage. In use, the motion actuator assembly may be configured to effectuate a fluid communication between one or more components, e.g., a sensor unit or an injector of the dispensing system, and a sample within a well of the sample carrier (e.g., multi-well plate).

In an exemplary embodiment, one or more sensor(s) may be adapted to sense changes in oxygen level and pH (proton concentration) of the cellular media associated with the metabolic activity of the cell sample in a well of the sample carrier (e.g., the multi-well plate). The stage, sensor, and dispensing system may cooperate to simultaneously measure a basal oxygen consumption rate and a basal extracellular acidification rate of the cell sample using the sensor. Thereafter, the dispensing system may be used to sequentially administer to the cell sample one or more agent. In an exemplary embodiment, the one or more agent may include mitochondrial ATP synthase inhibitor (Oligomycin A), mitochondrial uncoupling agent BAM15, and/or a mixture of mitochondrial Complex I and Complex III inhibitors (rotenone and antimycin A, respectively). The sensors may measure oxygen consumption rate and extracellular acidification rate, optionally substantially simultaneously, after each dispensing of the one or more agent. An additional agent, for example, a modulator reagent, can be optionally dispensed before the dispensing of described reagents or an extracellular membrane ionophore monensin can be injected after the injection of rotenone/antimycin A to the cells. The same measurements of oxygen consumption rate and extracellular acidification rate may be performed before and after each dispensing.

Components of the apparatus are further described in, e.g., U.S. Pat. No. 7,276,351 titled “Method and device for measuring multiple physiological properties of cells” and U.S. Pat. No. 8,658,349, titled “Cell analysis apparatus and method,” each of which is incorporated herein by reference in its entirety for all purposes.

One or more of the following features may be included. The sensor may be configured to analyze the constituent without disturbing the cells. The well may include any feature that allows the corresponding sensor unit plunger from the array of sensor units to move a defined position in the well to create a microchamber, e.g., a step, ring bump, lip. The sensor unit plunger or barrier may be adapted to stir the media prior to analysis of the constituent and/or after. The sensor may be a photoluminescent based sensor. The sensor may be, for example, a fluorescent sensor, a luminescent sensor, an ISFET sensor, a surface plasmon resonance sensor, a sensor based on an optical diffraction principle, a sensor based on a principle of Wood's anomaly, an acoustic sensor, or a microwave sensor. At least a portion of the well may be adapted to receive the sensor. The reduced volume of media effectuated by the plunger may include the sensor, and/or at least a portion of the barrier may include the sensor.

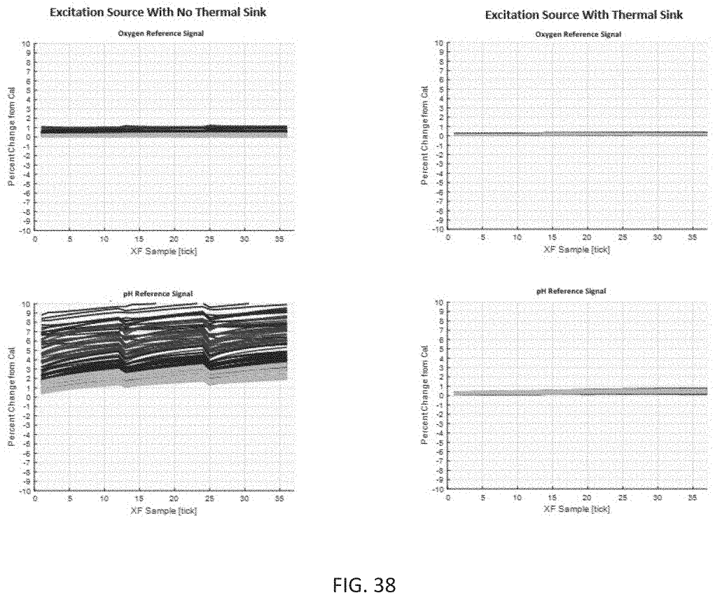

The instrument may comprise a light source, e.g., a fluorescent light, light emitting diode (LED), or laser, configured to excite a sensor of the sensor unit to generate a signal responsive to the target analyte or property being measured. In some embodiments, the light source may be configured to produce a reference signal. Fluctuations in intensity from the light source may be corrected proportionally to drift by monitoring the reference signal produced by the light source. The light source may be positioned on a thermally conductive printed circuit assembly configured to minimize drift from the light source. In some embodiments, the thermally conductive printed circuit assembly may be formed of a material configured to minimize drift generated by heat-induced fluctuations from the light source by at least 20%, e.g., at least 15%, 10%, or 5%. In some embodiments, the thermally conductive printed circuit assembly reduces drift by about 80%, 85%, 90%, 91%, 92%, 93%, 94%, 95%, 96%, 97%, 98%, and 99% FIG. 38 shows a reduced correction factor from 10% drift correction down to under 1%. In some embodiments, the thermally conductive printed circuit assembly is formed of a material configured to substantially minimize drift such that a reference signal is not needed, thereby reducing cost for the system.

In certain embodiments, the one or more sensors may be adapted to analyze (determine the presence or concentration of) an extracellular constituent in a well, such as CO₂, O₂, Ca⁺⁺, H⁺, or a consumed or secreted cellular metabolite. Analytes proportional to O₂ content include, for example, CO₂, O₂. Analytes proportional to pH of a sample include, for example, Ca⁺⁺, H⁺. More than one analyte, e.g., at least one analyte, may be measured to analyze the extracellular constituent.

The one or more sensors may be adapted to analyze a first extracellular constituent. In some embodiments, the one or more sensors may be adapted to analyze a plurality of extracellular constituents, e.g., more than one, more than two, more than three, more than four, or more constituents. Each sensor may analyze the plurality of constituents simultaneously. Each sensor may analyze the plurality of constituents individually, e.g., sequentially. The disclosure generally describes a sensor unit configured to analyze a first target analyte, e.g., at least one analyte proportional to O₂ content, and a second target analyte, e.g., at least one analyte proportional to pH value. However, it should be understood that the sensor unit may be configured to analyze additional or alternative target analytes.

In certain embodiments, the sensor is an optical sensor. The optical sensor may be a fluorescent or phosphorescent based sensor. The sensor may alternatively utilize solid-state, nanoparticulate, microparticulate, and/or magnetic sensors, or the like. For instance, solid state sensors may include one or more spots or films on the lid, base, projections, or combination thereof, where particle base sensors may generally be in solution or in suspension. Alternatively, in one aspect, particle based sensors can be loaded into cells or coated onto a surface. Nonetheless, such sensors can include optical, O₂, pH, temperature, CO₂, or combinations thereof.

Furthermore, in one aspect, the sensor can be an electrochemical, or potentiometric sensor. Additionally or alternatively, electrodes may also be included in the well in order to measure electrical characteristics, including impedance. Notwithstanding the senser selected, in one aspect, and as discussed above, it should be understood that the well or chamber may also contain one or more reference probes which generates a signal of known value for instrument calibration in the form of any of the sensors discussed above.

One exemplary sensor unit is an oxygen-sensitive photoluminescent dye. The photoluminescent dye may be selected from any oxygen sensitive photoluminescent dye. A suitable dye may be selected based on the intended use of the probe. A non-exhaustive list of suitable oxygen sensitive photoluminescent dyes includes specifically, but not exclusively, ruthenium(II)-bipyridyl and ruthenium(II)-diphenylphenanothroline complexes, porphyrin-ketones such as platinum(II)-octaethylporphine-ketone, platinum(II)-porphyrin such as platinum(II)-tetrakis(pentafluorophenyl)porphine, palladium(II)-porphyrin such as palladium(II)-tetrakis(pentafluorophenyl)porphine, phosphorescent metallocomplexes of tetrabenzoporphyrins, chlorins, azaporphyrins, and long-decay luminescent complexes of iridium(III) or osmium(II).

Typically, in such embodiments, the hydrophobic oxygen-sensitive photoluminescent dye may be compounded with a suitable oxygen-permeable and hydrophobic carrier matrix. A suitable oxygen-permeable hydrophobic carrier matrix may be selected based on the nature of the intended biological sample to be tested and the selected dye. A non-exhaustive list of suitable polymers for use as the oxygen-permeable hydrophobic carrier matrix includes specifically, but not exclusively, polystryrene, polycarbonate, polysulfone, polyvinyl chloride and some co-polymers. An alternative is to stain oxygen-permeable micro-beads with an oxygen-sensitive photoluminescent dye, mix the stained beads with silicone or polyurethane, and applying the mixture as a polymeric coating.

Regardless of the type of solid sensor selected, in one aspect for example only, the sensor may be embedded in a permeable medium, such as a permeable medium selected from hydrogel, silicone, and matrigel. In some aspects, the sensor is attached at least one of the projections by solidifying or removing the medium (such as by drying, curing, cooling, evaporating or other technique). The solid-state sensor can be applied by dipping or spotting the distal end of at least one of projections in a mixture of a fluorescent indicator in a medium.

However, it should be appreciated that in certain aspects, the sensor can be spotted or dipped onto all or a portion of one or more of the projections. It should further be appreciated that in certain aspects, the sensor can be removably connectable to the body of one or more projections of the assembly. It should further be appreciated that in certain aspects, the sensors can be integrally formed with one or more projections. Integrally forming the sensors on one or a plurality of projections can be achieved by one or more techniques, such as vapor deposition, chemical coating, spin coating, dipping, and robotic spotting.

The dispensing system may include one or more injectors configured to introduce fluids or agents independently and selectively into at least one well (e.g., each well). In some embodiments, the dispensing system may include an array of injectors, e.g., at least one injector positioned to correspond with at least one well (e.g., each well) of the well plate. In some embodiments, the dispensing system may comprise one or more movable injectors, each configured to introduce fluids or agents into a plurality of wells of the well plate.

In certain embodiments, in order to actuate movement of the one or more injectors, e.g., across a plurality of wells, the instrument may comprise an injector motion actuator assembly positioned to drive the at least one injector. The injector motion actuator assembly may drive the one or more injector across a row of wells, a column of wells, or in a pre-selected pattern across any configuration of wells.

Thus, the instrument may have 1, 2, 3, 4, 5, 6, 7, 8, 9, or 10 moveable injectors positioned to be driven across a plurality of wells, row of wells, or column of wells. Alternatively, the dispensing system may have an array of one or more injectors, e.g., 1, 2, 3, 4, 5, 6, 7, 8, 9, or 10 fixedly positioned to correspond with at least one well (e.g., each well). The instrument may have a ratio of wells to injectors of 1:1 to 1:384, e.g., 1:1, 1:2, 1:3, 1:4, 1:8, 1:12, 1:24, 1:36, 1:48, 1:64, 1:72, 1:96, 1:192, or 1:384. The instrument may have a ratio of injectors to wells of 1:1 to 1:384, e.g., 1:1, 1:2, 1:3, 1:4, 1:8, 1:12, 1:24, 1:36, 1:48, 1:64, 1:72, 1:96, 1:192, or 1:384.

The assembly and processes according to example aspects of the disclosure can be well suited to measuring constituents in all different types of samples, such as biological samples. In one aspect, for instance, the systems and processes according to example aspects of the disclosure can be used to measure one or more constituents or a parameter related to the constituent in cellular material. The one or more constituents may be contained in a medium surrounding the cells or can be contained within the cells themselves. In some embodiments, the biological sample being tested may contain cellular material derived from cells, such as cellular organelles, mitochondria, cellular extracts, cell products or byproducts, or conditioned medium. The measurements can be completed in a label-free manner.

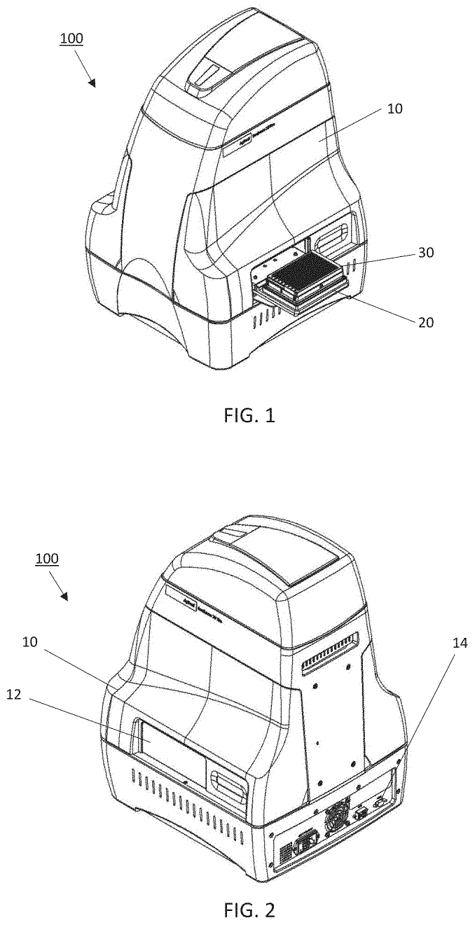

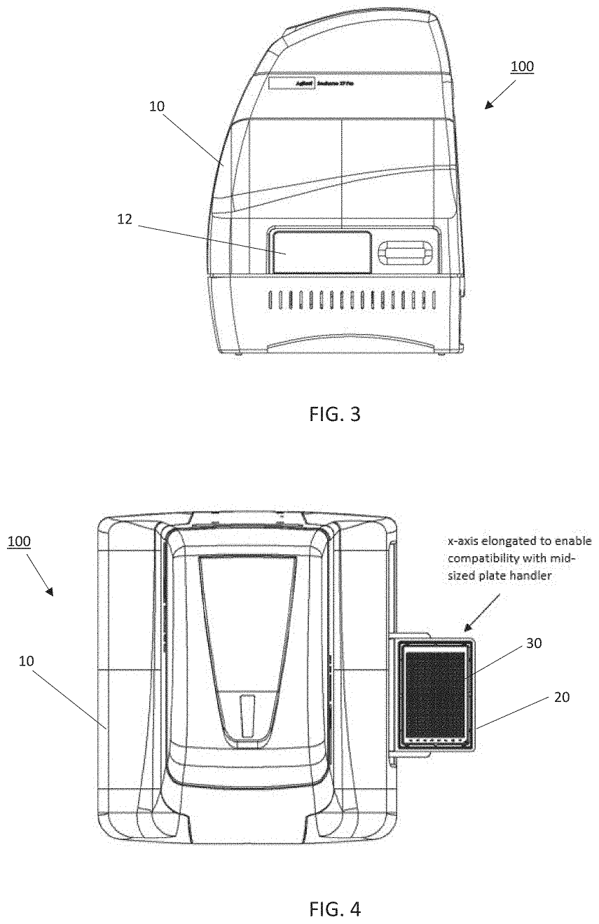

An exemplary analytical instrument is shown in FIGS. 1-4. As shown in FIGS. 1-4, the instrument or apparatus 100 includes a housing 10 having an opening on a side wall of the housing 10. The opening may optionally be closeable by a door 12. Within the housing 10 there is provided a stage 20 adapted to receive a sample carrier (e.g., a multi-well plate) 30. The stage 20 may be movable to be positioned within the housing 10 or exterior to the housing 10 through the opening by an x-axis actuator assembly. The door 12 may be shut when the stage 20 is positioned within the housing 10 for testing. The housing may include one or more electronic port 14 connectable to a computer and/or power source.

The electronic port 14 may be compatible with one or more of USB, mini-USB, HDMI, DVI, dual DVI, mini-DVI, micro-DVI, displayport, mini displayport, VGA, mini-VGA, RS-232, Ethernet/LAN, or any other electronic port capable of transmitting data. The apparatus shown in FIGS. 1-4 includes an electronic port 14, however, it should be noted that the apparatus may be connectable to an external computer by any means known in the art, for example, wireless fidelity network (WiFi), ultrahigh frequency radio waves (also known as Bluetooth®), or any other data transmitting connection. In embodiments, the apparatus may be connectable to an external computer through the cloud.

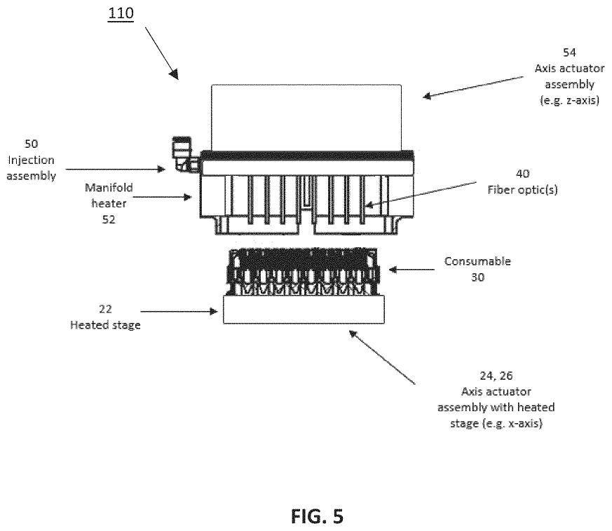

An exemplary assembly 110 is shown in FIG. 5. The assembly 110 may be contained within housing 10 shown in FIGS. 1-4. The assembly 110 includes components of a sensing system (e.g., fiber optic) 40 comprising an array of sensor units and dispensing system 50 comprising an array of injectors disposed on a manifold. In some embodiments, the manifold comprises holes where pressurized air is forced through a sensor cartridge comprising drug/compound/substance ports containing substances that correspond to the holes in the manifold and is ‘sealed’ by a gasket. A force is applied to the manifold which enables the substance to be delivered to the samples. One or more component of the manifold may be independently movable on a z-axis as directed by the z-axis actuator assembly 54 of a motion actuator assembly. Temperature of the manifold and/or cartridge may be controlled by manifold temperature controller 52. The assembly 110 includes stage 20 adapted to receive sample carrier (e.g., multi-well plate) 30 (with a cartridge shown on top). Temperature of the samples within sample carrier (e.g., multi-well plate) 30 is controlled by sample temperature control element 22. The stage 20 is movable along an x-axis as directed by the x-axis actuator assembly 24 of the motion actuator assembly. The motion actuator assembly also includes a y-axis actuator assembly 26 configured to move stage 20 along a y-axis.

The apparatus may include an automated measurement system. The apparatus may also include or be connectable to a computer, with the automated measurement system being in electrical communication with the computer. In certain embodiments, the apparatus may also include a controller for effecting the addition of one or more fluids or agents to one or more of the wells of the sample carrier (e.g., the microplate). The controller may operate the sensor to effectuate the sensing of one or more constituent in the one or more wells of the sample carrier (e.g., the microplate). The system may be in communication with the controller and the sensor via a graphical user interface residing on the computer. The graphical user interface may be configured to receive instructions for the design of a multi-well experiment in accordance with the methods disclosed herein, instruct the controller to execute the multi-well experiment, and to receive the data acquired by the sensor in response to the execution of the multi-well experiment.

In certain embodiments, the graphical user interface may include a plurality of display areas, each area being attributed to one of the wells. The graphical user interface may be configured to receive instructions written in respective areas attributed to one of the wells for the design of a multi-well experiment, and receive the data acquired by the sensors in response to the execution of the multi-well experiment for display in a respective area attributed to one of the wells. Thus, the methods, executable by the controller, may be independently and selectively applied to one or more wells through instruction from the graphic user interface.

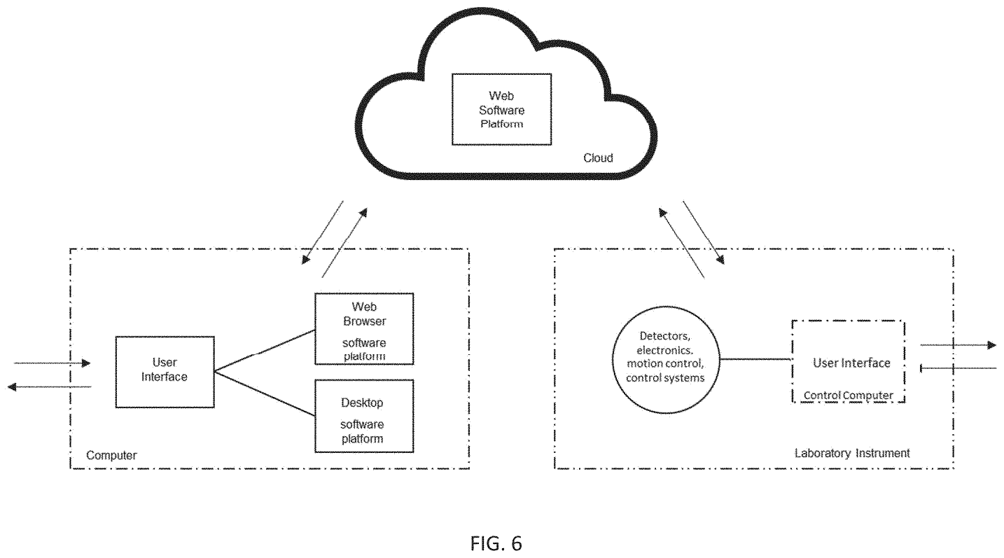

FIG. 6 shows an exemplary system including the analytical instrument (laboratory instrument) connectable to a cloud-based computing network and a computer through the cloud-based network. The analytical instrument includes detectors or sensor units and other electronics, such as the signal processing module and motion actuators. The detectors and electronics are controllable by one or more controller such as a motion controller operably connected to the motion actuator assembly and a control system operably connected to the sensing system and/or dispensing system. Protocols for the analytical instrument components may be provided through the user interface accessible on the computing device or cloud-based computing network. The user interface may be provided on a web browser software platform and/or on a desktop software platform. It should be noted that the desktop software platform may be provided on a desktop computer, laptop computer, and/or tablet or other mobile device. The web browser software platform may provide cloud-based data processing, cloud-based data storage, and/or the cloud-based connection between the computer and the analytical instrument. Other mechanisms for connecting to the cloud may be used, for example, desktop software or driver software. A data storage module may also be included in the system, for example, a local memory storage device, e.g., servers, external drives, portable drives, and/or a cloud-based memory storage device. The data storage module may store historical data, protocols, data processing algorithms, and/or controller executable instructions.

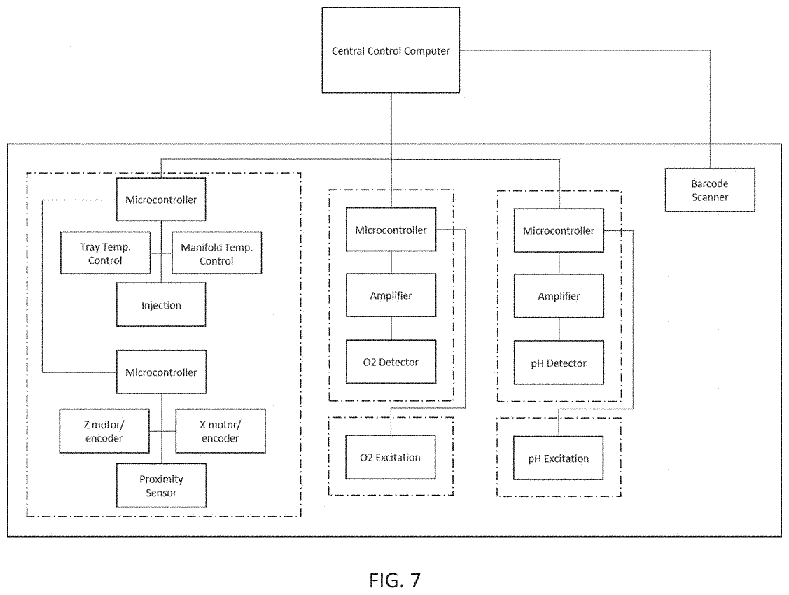

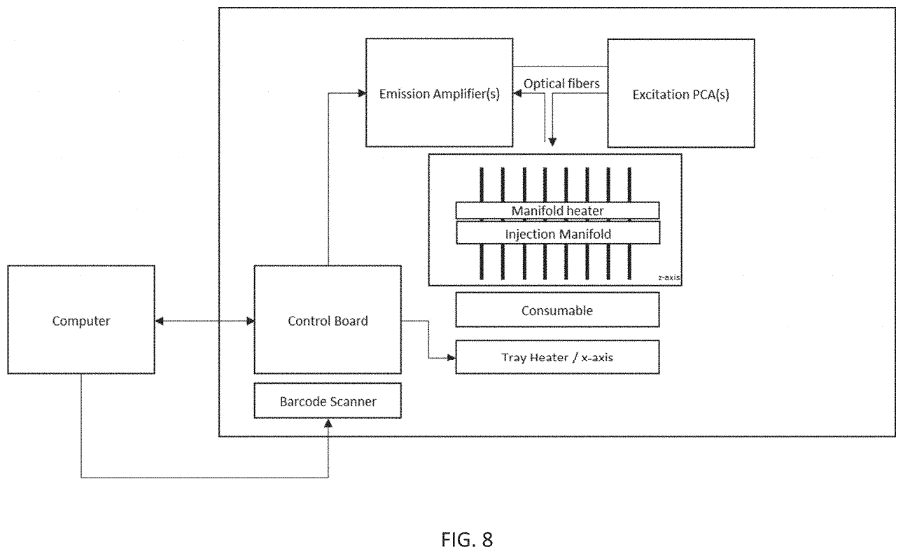

FIGS. 7-8 are schematic diagrams of the systems disclosed herein and electronic components shown in more detail. FIG. 7 is a diagram of the analytical instrument operably connected to a central control computer. The baseboard includes a microcontroller or system controller operably connected to temperature control elements for the manifold and tray (i.e., sample temperature control element) and a dispensing system or injection unit. A further microcontroller, also referred to as “motion controller” herein, is shown operably connecting the controller and motion actuator assembly including a z motor operating the z-axis actuator assembly and an x motor operating the x-axis and optionally y-axis actuator assembly. The apparatus described herein may comprise stepper motors with higher torque that improve precision in measurements over the life of the instrument and reduce the need to provide maintenance and/or replace motor components.

A proximity sensor is also provided as part of the motion actuator assembly configured to sense relative positioning of the stage or sample carrier (e.g., multi-well plate) and other instrument components, such as the sensor units and dispensing system injectors. The proximity sensor may be configured to generate a notification signal, and optionally pause a protocol, if a component is positioned within a predetermined distance from another component, e.g., a sensor unit within a predetermined distance from a corresponding well of the sample carrier. Additionally or alternatively, the proximity sensor may be configured to generate a notification signal, and optionally pause a protocol, if the opening on the side wall of the housing is ajar and/or external light is detected within the housing.

The system may also contain a stall sensing module programmed to generate a notification signal, and optionally pause a protocol, e.g., halt motor movement, if a predetermined protocol step is not completed within a predetermined time interval. The stall sensing module may be configured to detect stalls through the use of encoders. For instance, the encoders may operate by looking for timing related delays in the encoder travel and flag a stall.

The diagram of FIG. 7 also includes sensing units for an O₂ analyte and a pH analyte operably connected to a signal processing module comprising an amplifier and microcontroller configured to receive and amplify signals from the sensor units. The signal processing module is further operably connected to the system controller and central control computer. The system further includes a barcode scanner configured to scan a barcode encoding information operably transmittable to the central control computer.

FIG. 8 is a schematic diagram of the system showing the computer operably connectable to the system control board or system controller and barcode scanner. The barcode scanner is configured to decode and transmit information from the barcode to the computer. The system controller is operably connected to the tray heater or sample temperature control element configured to control temperature of the consumable or samples within a sample carrier (e.g., a multi-well plate). The system controller is also operably connected to the emission amplifier or signal processing module. The signal processing module is operably connected to the optical fibers or sensor units. In some embodiments, the system controller is also operably connected to the manifold heater or manifold temperature control element configured to control temperature of the injection manifold or dispensing system. Optionally, a separate system controller may be provided operably connected to the manifold heater or manifold temperature control element.

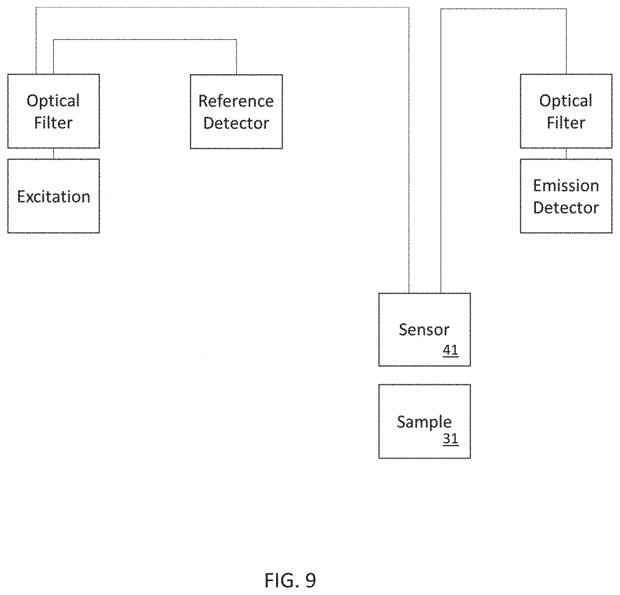

FIG. 9 shows an exemplary sensor unit 41 deployed within a well 31. The exemplary sensor unit 31 is a fluorescent sensor. Disposed on the surface of the well 31 there may be a fluorophore having fluorescent properties dependent on at least one of the presence and the concentration of a constituent in the well 31. The sensor unit 41 may include a housing for receiving a wave guide for at least one of stimulating the fluorophore and for receiving fluorescent emissions from the fluorophore.

The disclosure provides a method, apparatus, and measurement system for adding a test compound to a well and measuring a constituent of the well with a sensor. The method may be performed as a high-throughput assay, by adding one or more test compound to one or more wells, respectively, or multiple of the same or different test compounds to multiple wells of a sample carrier (e.g., a microplate). In certain embodiments, the test compound is introduced while a sensor probe remains in equilibrium with, e.g., remains submerged within, the liquid contained within at least one well (e.g., each well). In such embodiments, because the sensor probe remains submerged during compound delivery, equilibration time may be reduced. Thus, a system and a method are provided for storing and dispensing a single preselected test compound, or preselected concentration of the compound per well.

In certain embodiments, the apparatus and method store and deliver one or more test compounds or target agents per well. Test compounds may be delivered using a supply of compressed gas from a remote source to actuate the compound delivery. In certain embodiments, both the sensor probe and test compound delivery structure are incorporated within a single disposable cartridge. A pneumatic multiplexer is also described that, when temporarily attached to the cartridge, allows a single actuator to initiate the delivery of test compound from multiple ports using a supply of compressed gas from a remote source.

In one aspect, there is provided a cartridge adapted to mate with a sample carrier (e.g., a multi-well plate) having a plurality of wells. The cartridge may include a substantially planar element having a plurality of regions corresponding to a common number of respective openings of the wells in the sample carrier (e.g., the multi-well plate). At least one port may be formed in the cartridge in at least one region, the port being adapted to deliver a test fluid, e.g., an aqueous solution of a candidate drug/compound/substance or other agent, to the respective well. The cartridge may also include at least one of a) a sensor or portion thereof adapted to analyze a constituent in a well and b) an aperture adapted to receive a sensor located in a sub region of the at least one region of the cartridge.

Components and features of the cartridge are further described in, e.g., U.S. Pat. No. 9,170,255 titled “Cell analysis apparatus and method,” which is incorporated herein by reference in its entirety for all purposes.

The apparatus may include an elevator mechanism adapted to move the cartridge relative to the stage or the plate to dispose the sensor in the well, typically multiple sensors in multiple wells simultaneously. A pressure source adapted to be mated fluidically with the cartridge may be provided, to deliver the test fluid from a port in the cartridge to a well. The apparatus may also include a multiplexer disposed between the pressure source and the cartridge, the multiplexer being adapted to be in fluidic communication with a plurality of ports formed in the cartridge. The multiplexer may be in fluidic communication selectively with exclusive sets of ports formed in the cartridge. A controller may be provided to control the elevator mechanism, the multiplexer, and/or the pressure source to enable delivery of test fluid from a given port or set of ports to a corresponding well or set of wells when an associated sensor is disposed in the well. The controller may be in communication with the computer or graphical interface, as previously described.

In certain exemplary embodiments, the aperture of the cartridge adapted to receive the sensor may comprise a sensor sleeve structure having a surface proximal to a well of the sample carrier (e.g., the multi-well plate). Disposed on the surface may be a fluorophore having fluorescent properties dependent on at least one of the presence and the concentration of a constituent in the well. The sensor sleeve may include an elongate housing for receiving a wave guide for at least one of stimulating the fluorophore and for receiving fluorescent emissions from the fluorophore.

An array of sensors corresponding to an array of wells may be integral with the cartridge, but may also be separate elements mated with and disposed within apertures formed in the cartridge. The sensor array may be mounted compliantly relative to the well plate.

Methods of analyzing cells with the apparatus disclosed herein are provided. The methods may be employed to measure cells disposed in media in a sample carrier (e.g., a multi-well plate). The method may include one or more of disposing as least a portion of a sensor in media in a well in the sample carrier (e.g., the multi-well plate), analyzing a constituent related to the cells within the media in the well, delivering a test fluid to the well while the sensor remains disposed in the media in the well, and further analyzing the constituent to determine any change therein. In certain embodiments, one or more constituent may be analyzed substantially simultaneously. In particular, a rate change of the one or more constituent may be measured over the assay time, for example, to determine metabolic or other activity of the cell sample.

The analyzing step may include analyzing respective constituents related to respective cells within media in respective wells. The respective constituents may be the same constituent. The delivering step may include delivering respective test fluids or target agents to the respective wells while respective sensors remain disposed within media in respective wells. The respective test fluids or agents may include the same test fluid or agent.

The step of analyzing may include analyzing respective constituents related to respective cells within media in respective wells to determine any respective changes therein. The delivering step and the further analyzing step may be repeated. A different test fluid or agent or an additional aliquot of the same test fluid or agent may be delivered between measurements. The method may include substantially maintaining equilibration between the sensor and the media during the delivery step or maintaining thermal equilibrium between the test fluid and the media during the delivery step.

The methods may include controlling temperature and/or an environment of the cell samples before, during, and/or after the analyzing step. In certain embodiments, the methods may include controlling temperature and/or an environment of the cell samples throughout performance of the analytical method. Controlling environment may include, for example, controlling relative humidity (RH) and/or a composition of the environmental gas, such as N₂, O₂, and/or CO₂ concentration. For example, in certain embodiments, controlling environment may include inducing a hypoxic environment by purging the air with N₂ gas.

The method may further include imaging or scanning the samples during the analyzing step, during the delivering step, and/or subsequent to the analyzing step and/or the delivering step.

The devices and methods disclosed herein may be used to analyze biological samples, also referred to as cell samples herein. In particular, the devices and methods disclosed herein may be used to analyze live cell samples. The samples may comprise or be in the form of one or more of loose cells, cell constructs, loose tissue, tissue constructs, organelles, enzymes, cell products or byproducts, and conditioned medium. The cell samples may comprise mammalian cells or tissue. The cell samples may comprise non-mammalian cells or tissue. The samples may comprise animal cells or tissue. The samples may comprise insect cells or tissue. The samples may comprise plant cells or tissue, e.g., seeds, pods, or other plant materials. The samples may comprise single-celled organisms, e.g., microorganisms. In certain exemplary embodiments, the sample may comprise whole plant or animal model tissues, e.g., zebrafish, C. elegans, drosophila.

The biological material being analyzed may comprise a cellular material. The biological material may contain living cells comprising bacteria cells, fungus cells, yeast cells, prokaryotic cells, eukaryotic cells, insect cells, or the like. The cells can be animal cells, human cells, immune cells, or immortal cells.

Exemplary cells include human T cells (CD4+, Pan CD3+, CD8+, PBMC, e.g., naïve, activated, effector and memory), mouse T cells (spleen derived CD8 naive and activated), immortalized mouse myoblast cells (e.g., C2C12), Jurkat cells, lung cancer cell models (A549, PC9, H1373), leukemia cancer cell model (THP-1), human hepatoma cells (e.g., HepG2), human epidermoid carcinoma cells (e.g., A431), and analysis of entire organisms, such as zebrafish, C. elegans, and drosophila. Certain aspects of the devices and methods disclosed herein enable analysis of live cells requiring a temperature of 28° C.-40° C., without the need to place the instrument in a temperature-controlled room.

The devices and methods disclosed herein may be employed to facilitate research in the fields of cancer, immunology, toxicology, drug/compound/substance discover, and immunotherapy, among others.

In one aspect, the cell sample is obtained or derived from a subject, such as a human or non-human animal. In one aspect, the subject is a mouse, which, in an aspect, has, or is at risk of having, a disorder. Nonetheless, in an aspect, the cell sample can include a primary cell, a cell isolated or harvested directly from a living tissue or organ, a cultured cell, and/or an immortalized cell. For instance, the cell sample can include a primary cell, or a cell isolated or harvested directly from a living tissue or organ, and then cultured ex vivo. In an aspect, the cell sample includes a cell that has been modified, e.g., genetically engineered for heterologous expression of a gene of interest, and/or genetically engineered for inhibition expression of a gene, such as cells from knock out mouse or CRISPR KO libraries. Nonetheless, in one aspect, the cell sample includes a stem cell or a cell derived from a stem cell. Nonetheless, regardless of the cell used, in one aspect, the cell sample includes a medium, e.g., a culture medium or a growth medium, where the cell can be disposed in the medium. Furthermore, as would be understood, in one aspect, the cell sample comprises a plurality of cells, e.g., a plurality of cells described herein.

The cells being tested can comprise any suitable cell sample, including but not limited to cultured cells, primary cells, human cells, neurons, T cells, B cells, epithelial cells, muscle cells, stem cells, induced pluripotent stem cells, immortalized cells, pathogen-infected cells, bacterial cells, fungal cells, plant cells, archaeal cells, mammalian cells, bird cells, insect cells, reptile cells, amphibian cells, and the like. The cells being tested may also comprise a monolayer of cells, two-dimensional cell samples, three-dimensional cell samples, such as tissue samples, cell spheroids, organoids, biopsied samples, cell scaffolds, organs-on-a-chip, and the like. Examples of parameters that may be measured and are related to the above cell functions include carbon dioxide concentration, oxygen concentration or oxygen partial pressure, calcium ions, hydrogen ions, and the like. However, in one aspect, the measured parameter is oxygen concentration, such as oxygen consumption. Through these tests, one can gain an understanding of what drives cell phenotype and function and/or an accurate picture of the cellular environment or microenvironment.

The assembly and process according to example aspects of the present disclosure can be used to measure live cell metabolic data, or (micro)environmental conditions of any viable cell. The cellular material being tested, for instance, can comprise bacteria cells, fungus cells, yeast cells, prokaryotic cells, eukaryotic cells, and the like. Cells that can be tested include mammalian cells including animal cells and human cells. Particular cells that can be tested include cancer cells, immune cells, immortal cells, primary cells, induced pluripotent stem cells, cells infected with viral or bacterial pathogens, and the like.

For example, in one aspect, the assembly and process according to example aspects of the disclosure can be used to assist in immunotherapy. Immunotherapy is a type of treatment that bolsters a patient's immune system for fighting cancer, infections, and other diseases. Immunotherapy processes, for instance, can include adoptive cell based therapies, such as the production of T cells, Natural Killer (NK) cells, monocytes, macrophages, combinations thereof and the like. During T cell therapy, for instance, T cells are removed from a patient's blood. The T cells are then sent to a bioreactor and expanded or cultivated. In addition, the T cells can be changed so that they have specific proteins called receptors. The receptors on the T cells are designed to recognize and target unwanted cells in the body, such as cancer cells. The modified T cells are cultivated in a bioreactor to achieve a certain cell density and then supplied to a patient's body for fighting cancer or other diseases. T cell therapy can also be referred to as adoptive T cell therapy or T-cell transfer therapy, one example of which is referred to chimeric antigen receptor (CAR) T cell therapy. The use of T cells for adoptive T cell therapy or T-cell transfer therapy has recently proliferated due to great success in combating blood diseases. In some embodiments, aspects of the present invention may be used to monitor the health of T cells used in adoptive T cell therapy or T-cell transfer therapy. In some embodiments, aspects of the present invention may be used to monitor T cell activation, T cell exhaustion, T cell metabolism including of starting material and modified products, and the like.

NK cells are a type of cytotoxic lymphocyte that can seek out and destroy infected cells within the body. NK cells can display very fast immune reaction responses. Consequently, the use of NK cells in anticancer therapy has grown tremendously in interest and popularity. There is only a limited number of NK cells in the blood of a mammal, however, requiring that NK cells be grown to relatively high cell densities within bioreactors.

The culturing of cells, such as T cells, NK cells, or other mammalian cells, typically requires a somewhat complex process from inoculation to use in patients. The assembly and process of the present disclosure can be used to monitor cell metabolism during any point in the culturing process to ensure that the cells are healthy, and/or have the desired metabolic phenotype, and that the media in which the cells are growing contains an optimized level of nutrients. The system and process, for instance, can be used to make adjustments for assuring the metabolic fitness of the cells as they are growing.

In addition to immune cells, the metabolism of cancer cells can also be monitored for providing an understanding of which nutrients fuel the cancer cells. For example, the assembly and process according to example aspects of the present disclosure can reveal mechanisms or components that impact the metabolism of the cancer cells for inhibiting growth. The assembly and process according to example aspects of the present disclosure can also be used to determine the speed at which the cancer cells may proliferate. The system and process of the present disclosure is also well suited for use in toxicology. For instance, the process and assembly of the present disclosure can be used to detect mitochondrial liabilities among potential therapeutics. The risk of mitochondrial toxicity, for instance, can be assessed with high specificity and sensitivity. In this manner, the mechanism of action of some mitochondrial toxicants can be determined.

Temperature Control

The apparatus described herein includes one or more temperature control elements designed to reduce temperature gradient between outer (e.g., border) and inner wells of sample carrier (e.g., the multi-well plate). A sample temperature control element and a manifold temperature control element are described herein. The temperature control elements may be designed to control temperature independently from one another. The temperature control elements are generally formed of a temperature conductive material which is optionally positioned in close proximity or in direct contact with one or more components, such as the sample carrier (e.g., the multi-well plate), sensor units, and/or injectors. For instance, the sample temperature control element may be dimensioned to fit the sample carrier (e.g., the multi-well plate). The manifold temperature control element may be dimensioned to fit the sensors, injectors, and/or the cartridge and, optionally, cover the sample carrier (e.g., the multi-well plate) when the cartridge is positioned to mate with the sample carrier (e.g., the multi-well plate), e.g., when the sensor units and/or injectors are in fluid communication with the wells of the sample carrier (e.g., the multi-well plate). In some embodiments, a micro-environment that includes a manifold and heater and a heated component that surrounds the sensor cartridge and a tray heater that is in direct contact with the sample carrier is formed, which allows for maintaining the temperature for an extended period of time. The manifold temperature control element may be configured to mate with the sample temperature control element to cover the sample carrier (e.g., the multi-well plate).

The design of the temperature control elements forms a controlled temperature zone or microenvironment within the instrument. The controlled temperature zone generally comprises the array of wells of the sample carrier. In particular, the controlled temperature zone does not comprise a headspace, or a substantially large portion of the headspace, of the housing, for example, temperature control does not extend to the entire internal chamber of the instrument, such that temperature of components outside the controlled temperature zone is not substantially altered, e.g., increased or decreased, by activation of the temperature control elements. In some embodiments, a volume of the controlled temperature zone does not exceed a volume of the sample carrier by more than 1, 2, 3, 4, 5, 6, 7, 8, 9, or 10-fold. In some embodiments, a volume of the controlled temperature zone does not exceed 10%, e.g., 9%, 8%, 7%, 6%, 5%, 4%, 3%, 2%, or 1%, of a volume of the housing.

It was surprisingly discovered that the design of the temperature control element allowed operation of the instrument at a temperature lower than expected, for example, at a temperature of 8° C. or lower, as compared to the typical low end operational temperature of 12° C. The low end of operational temperature is sometimes limited by heat produced by the system components, such as motors or motor control components, power supplies, circuit boards, and light sources. The lower operational temperature allows the instrument to be used to examine sample types that could previously not be examined with such a device, e.g., zebrafish, whole cell organisms, or non-mammalian cells. Thus, in some embodiments, the temperature control element may control temperature of the sample within at least one well (e.g., each well) to be less than 12° C., for example, less than 10, 9, 8, 7, 6, 5, 4, 3, 2, or 1° C.

The creation of a controlled temperature zone or microenvironment generally allows the instrument to bring the temperature of samples within at least one well (e.g., each well) of the sample carrier to be within a predetermined range of a target temperature within about 5 hours, 3 hours, 1 hour, 45 minutes, 30 minutes, 15 minutes, 10 minutes, 5 minutes, 3 minutes, or 1 minute of activation of the temperature control element and/or introduction of the sample carrier into the controlled temperature zone.

Furthermore, the design of the temperature control elements enables the instrument to achieve temperature uniformity and a greater range of operational temperatures than previous designs. The greater operational temperature range allows the instrument to be used with a greater variety of cell types, such as non-mammalian cells which may require lower or higher temperatures than previously achievable, improving viability during the assay. The greater operational temperature and temperature control elements may improve sensitivity of the sensing units, for example, allowing the device to have a lower OCR detection limit than previous instruments. In some embodiments, the uniformity and/or precision of the measurements are improved.

The manifold temperature control element may be configured to control temperature of the target agent and/or sensor units to be within 3° C., e.g., 2° C., 1° C., 0.6° C., 0.5° C., 0.4° C., 0.3° C., 0.2° C., or 0.1° C., of another injector and/or sensor unit. In certain embodiments, the manifold temperature control element may be configured to control temperature of the target agent and/or sensor units and the sample temperature control element is configured to control temperature of the samples within the array of wells of the sample carrier to be within 3° C., e.g., 2° C., 1° C., 0.6° C., 0.5° C., 0.4° C., 0.3° C., 0.2° C., or 0.1° C., of one another. Thus, the temperature control elements disclosed herein may generally maintain uniformity of temperature between different samples in the well plate, e.g., internal and border samples of the well plate, and/or between the cartridge components and their corresponding samples in the well plate.

In some embodiments, the sample temperature control element is configured to control the temperature of samples within at least one well (e.g., each well) of the sample carrier to be within a predetermined range. Exemplary predetermined ranges include 0° C.-70° C. above ambient temperature, e.g., 8° C.-20° C. above ambient temperature, e.g., 0, 1, 2, 3, 4, 5, 6, 7, 8, 9, 10, 11, 12, 13, 14, 15, 16, 17, 18, 19, 20, 30, 40, 50, 60, or 70° C. above ambient temperature. In some embodiments, the sample temperature control element is configured to control the temperature of samples, e.g., two identical or substantially identical samples, within at least one well (e.g., each well) of the sample carrier such that a sensor signal in response to a level, production, or consumption of a target analyte does not differ more than a predetermined amount between two identical or substantially identical samples, for instance, does not differ more than 10%, e.g., 5%, 3%, 1% or 0.1% between the two identical or substantially identical samples, e.g., when the samples are analyzed under the same or substantially the same conditions. In particular, the temperature control element may be configured to reduce or inhibit fluctuations in sensor readings, e.g., photoluminescence sensor readings, cell metabolism and other functions, and/or analyte concentration that may be produced as a result of temperature differentials.

The design of the temperature control elements reduces evaporation of the samples during execution of the protocol. Evaporation can affect cellular function when it is severe enough to change the concentration of analytes in the media. Uniformity in temperature achieved by the sample temperature control element and/or the manifold temperature control element has shown a reduced evaporation of the sample as compared to conventional devices. In some embodiments, the temperature control element may be configured to control evaporation of the samples within the array of wells to be less than 25%, e.g., less than 20%, 15%, 12%, 10%, 9%, 8%, 7%, 6%, 5%, 4%, 3%, 2%, or 1%. Evaporation may be controlled by such percentages for long duration assay, such as a 6-hour assay, 8-hour assay, 10-hour assay, or longer. Furthermore, the sample carrier (e.g., the multi-well plate) may be designed to reduce evaporation during the cell culture and incubation process.

It was surprisingly found that the design of the temperature control elements provides a lower detection limit of O₂ and improved precision of measurements. For example, the analytical instrument disclosed herein may have an OCR detection range of 2000 pmol/min to 0.01 pmol/min, e.g., 700 pmol/min to 0.01 pmol/min, e.g., 50 pmol/min to 0.01 pmol/min. In some embodiments, the analytical instrument may have an improved lower OCR detection limit of less than 50 pmol/min, e.g., less than 40 pmol/min, 30 pmol/min, 20 pmol/min, 10 pmol/min, 5 pmol/min, 3 pmol/min, 1 pmol/min, 0.1 pmol/min, or 0.01 pmol/min.

Additionally, the design of the temperature control elements may reduce, limit, or inhibit differential (gradient) diffusion of gases in the sample carrier, cartridge, and/or internal environment near the sample carrier or controlled temperature zone. The temperature control elements may be configured to control, e.g., reduce, limit, or inhibit, the diffusion of gases inside the controlled temperature zone, cartridge, well plate, such that a composition of gases in the environment does not vary significantly during the assay, e.g., does not vary more than 1%, 2%, 4%, 6%, 8%, 10%, 12%, 14%, 16%, 18%, or 20% during the assay.

Environmental Control

The apparatus described herein may include or be associated with one or more environmental control module designed to control the environment surrounding the sample carrier (e.g., the multi-well plate). The environmental control module may be designed to control environmental gas and/or relative humidity (RH) of the environment surrounding the samples. For example, the environmental control module may be configured to control one or more of N₂, O₂, and CO₂ concentration of the gas surrounding the samples. RH may be increased or decreased by the environmental control module. For example, RH may be decreased to less than 75%, 65%, 55%, 45%, or 35% or RH may be increased to greater than 65%, 75%, 85%, or 95%. The environmental control module may enable use of the instrument for ischemia/reperfusion modelling and other controlled gas experiments.

The environmental control module may comprise a source of a gas, e.g., one or more of N₂, O₂, and CO₂, fluidly connected to the sample carrier. The environmental control module may form a controlled environment zone which comprises the array of wells of the sample carrier. The controlled environment zone may be open or closed to the ambient environment. The environmental control module may comprise a pump or fan configured to direct gases to or clear gases from the well plate.

In certain embodiments, the environmental control module is incorporated in the instrument. The environment may be formed by moving a heated component to surround or cover or enclose the heated sample carrier. This heated component may be made of thermally conductive material, e.g. metals, aluminum, steel, etc. These thermally conductive materials may be anodized to reduce/eliminate electrical conductivity. The thermally conductive heated component may also block stray light (ambient light). In some embodiments, the container is substantially enclosed such that there is minimal air flow. The sealed container may house the well plate, for example, the stage holding the well plate. In some embodiments, the container may house the cartridge with the well plate. To form the controlled environment zone, the sealed container may be fluidly connected to the source of gas and purged with one or more selected gas accordingly.

In certain embodiments, the environmental control module is associated with the instrument. For example, in some embodiments, the instrument may be placed in a gas-controlled incubator or hypoxia chamber. Thus, the instrument may be configured for use within a gas-controlled environment, e.g., formed of materials suitable for use within a gas-controlled environment, such as materials with low gas solubility.

The environmental control module may be integrated with system software, e.g., operably connected to the controller and/or system processor. The software may be programmed to cycle the environmental control module in accordance with a selected protocol.

The environmental control module can be integrated with system software, e.g., operably connected to the controller and/or system processor taking inputs from measurements of the cellular microenvironment (e.g., intracellular O₂, pericellular O₂, or O₂ measurements proximate to the cell sample), thereby allowing environmental control to deliver a target cellular microenvironment. The software can be programmed to cycle the environmental control module to deliver the target microenvironment in accordance with a selected protocol.

Signal Processing Module

High impedance transimpedance amplifiers are susceptible to parasitic current paths. Such parasitic current paths may be caused by contamination on the surface due to flux residue or surface cleaners from soldering and manufacturing. Parasitic current paths may also be exacerbated in high humidity environments and absorption of moisture in the dielectric material used to insulate conductive paths.

The apparatus disclosed herein is designed to reduce parasitic current paths by including a signal processing module capable of operating at high relative humidity, for example, 75%, 85%, or even 95% relative humidity. It was unexpectedly discovered that performance of the signal processing module at high relative humidity allows assays and experiments to be performed on the instrument for a longer duration. In some embodiments, performing assays for a longer duration requires higher humidity and/or longer humidity times which in turn requires electronic circuitry in the apparatus that is more robust to operating at higher humidity and/or longer humidity times. Thus, real time cellular data may be collected from the cell samples and assays may be conducted for more than 1 hour, 2 hours, 3 hours, 4 hours, 5 hours, 6 hours, 7 hours, 8 hours, 9 hours, 10, hours, 11 hours, 12 hours or more, without negatively impacting sensitivity of the sensor units.

The signal processing module is a processor operatively connected to the array of sensor units, configured to receive and amplify the signals from the sensor units. The signal processing module may receive and amplify the plurality signals from the array of sensor unit simultaneously or individually, e.g., sequentially. In some embodiments, the signal processing module may be able to adjust amplification of signal to acquire data at a faster or slower rate, e.g., reduce amplification to increase acquisition speed. In some embodiments, the signal processing module is configured to operate with reduced parasitic current, e.g., reduced interference, dark currents, or noise, associated with the detection and/or amplification of the signals from the sensor units.

The signal processing module may be configured to detect the signals using time-based detection or intensity-based detection. Briefly, the radiation emitted by an excited probe can be measured in intensity units and/or lifetime/time-domain (including, for example, rate of decay, phase shift, or anisotropy detection). Intensity-based detection may include detecting and/or processing ratiometric measurements. Briefly, the measurement may include an analyte-sensitive signal measurement and an analyte-insensitive or largely analyte-insensitive reference measurement. The ratio between the references may be incorporated to facilitate ratiometric assessment of analyte flux or concentration.

In some embodiments, the signal processing module comprises a printed circuit assembly formed of an insulating material having a high dielectric constant. In some embodiments, the signal processing module may comprise one or more light-sensitive components, such as semiconductor diode, photomultiplier tube, avalanche photodiode, CMOS sensor, CCD, etc. In some embodiments, the one or more light-sensitive component may be connected to a transimpedance amplifier. In some embodiments, the signal processing module comprises a printed circuit assembly having a transimpedance amplifier including grounded guard traces. In some embodiments, the signal processing module comprises a printed circuit assembly having an integrator design. In some embodiments, signal processing module comprises a printed circuit assembly having an operational amplifier design. In some embodiments, the signal processing module comprises a printed circuit assembly formed of surface mount components, e.g., substantially free of secondary hand soldered high gain components. In some embodiments, the signal processing module comprises a printed circuit assembly comprising a thermally conductive excitation source, optionally wherein the thermally conductive excitation source is in thermal communication, e.g., thermal contact, with a thermal sink. The thermally conductive excitation source may be any excitation source that changes intensity with respect to temperature, e.g., a laser diode or light emitting diode (LED). In some embodiments, the signal processing module comprises a printed circuit assembly having an integrator design. In some embodiments, the signal processing module comprises a printed circuit assembly having an operational amplifier design.

It was surprisingly discovered that the design of the thermally conductive excitation source reduced the thermal drift significantly such that less reference correction is generally required, which may reduce correction errors and thereby improve precision of measurements (FIG. 38). The data shown in the graphs of FIG. 38 demonstrates that reduce in thermal drift after inclusion of a thermally conductive excitation source. In some embodiments, the improved design of the thermally conductive excitation source may alleviate (or remove) the need to include a reference signal detector, reducing complexity of the fiber optic routing and cost of the instrument while achieving similar and/or improved performance. Thus, in some embodiments, the design of the signal processing module removes the need for a reference signal detector and/or for a light source configured to produce a reference signal. The instrument may be free of the reference signal detector.

Components and features of the signal processing module are further described in Kester et al. “Section 5: High Impedance Sensors,” (Appendix) and incorporated herein by reference in its entirety for all purposes.

Optical Module

The apparatus may further comprise an optical module positioned to image or scan the samples within the sample carrier (e.g., the multi-well plate). The optical module may be positioned within the housing. The optical module may be operatively connected to the controller. The optical module may be controlled or operated via the graphical user interface. Furthermore, images or scans obtained by the optical module may be reviewed and/or recorded via the graphical user interface, optionally in real time. Thus, in some embodiments, the optical module is operatively connected to the computer and the computer is configured to display and/or record the image or scan of the samples in real time.

Cell based assays, and in particular live cell assays, are becoming more popular in the field of life science research. Sample carriers (e.g., microplates) are increasingly used as vessels for investigation of the cell growth process by qualitative and quantitative means. Often the work with cells is performed by a researcher utilizing multiple dedicated instruments.

Photoluminescence, e.g., fluorescence and/or phosphorescence, reading with instrumentation that has a light beam diameter sufficiently large to obtain a representative measurement of total well fluorescence, or of beam size to perform an area scanning and mapping of the signal across the well, can be accomplished with a dedicated conventional fluorescence reader or with a multi-detection reader. Most of the instruments provide incubation of the plate, fluid injection, and also allow an option of a gas control (CO2 and/or O2) similar to tissue culture incubators.

Much more information than just well's fluorescence signal level can be obtained from cells with the wide-field imaging modality. Laboratory microscopes, with bright field and phase contrast for unstained cells and fluorescence imaging for stained cells, are commonly used. Some instruments do allow for incubation chambers and environmental control. For sharper imaging or sectioning of 3D cell clusters like spheroids, confocal microscopy is used as a third instrumentation option.

Typically these instruments are purchased from various vendors, and a user may be forced to physically transfer the vessel, e.g., sample carrier (e.g., microplate) from one instrument to another instrument as needed, as well as to keep track of the overall sample analysis process and to collate and combine data from several instruments to obtain complete holistic analysis of the cell sample. Without robotics, it may be nearly impossible to properly conduct a long-term complex experiment or assay. Use of robotics further increases both analysis cost and complexity. The combination of non-imaging analysis modalities (fluorescence, absorbance and chemiluminescence), wide-field fluorescence imaging on a cell level, confocal fluorescence imaging, environmental control, and reagent injections in a single instrument would provide a complete holistic analysis solution, and would free the user from tedious sample carrier (e.g., microplate) handling, sample carrier (e.g., microplate) tracking and data transfer. Solutions for a combined system where the data obtained from individual instruments may be stored, collated and analyzed are described herein.

Multimode Measurement in the Cloud-Based System

In certain aspects, the devices and methods disclosed herein may be used to perform a complete analysis of a cell sample by qualitatively and quantitatively measuring different parameters of the same cell sample. The methods may include measuring the cellular metabolic function, bioenergetic poise, bioenergetic capacity, and bioenergetic work of the cell e.g., measuring O₂, CO₂, pH with the sensing subsystem. The methods may include visually observing a property of the sample, e.g., cell growth, cell health, cellular microenvironment, morphological changes, ultrastructural changes, marker expression, of the cell using the optical module, for example, by an automated cell imaging reader such as Cytation™ 5 or Cytation™ 7, as disclosed in U.S. Pat. No. 10,072,982, incorporated herein by reference in its entirety for all purposes. The methods may include detecting attachment, ultrastructural changes, growth, morphological changes, cell-cell interactions by impedance measurements using the sensing system or devices as described in U.S. Pat. Nos. 10,551,371; 10,539,523; 10,215,748; 10,067,121; 9,709,548; 9,612,234; 8,263,375; 8,041,515; 8,026,080; 7,470,533; 7,468,255, 7,560,269; 7,732,127; or U.S. Patent Application Publication No. 2018/0246019 and International Application Publication No. WO2021/202264A1, each of which is hereby incorporated by reference in its entirety for all purposes.

Cell-substrate impedance monitoring generally permits continuous real time monitoring of cells. Cell-substrate impedance monitoring may be used to assess the interaction between cells and electrodes, where changes in cell attachment, growth, morphology and motility over electrodes results in a detectable change. To this end, cell-substrate impedance monitoring is a useful tool that may be employed to assess cell proliferation and cytolysis. Coupled with real time cell analysis by impedance, the brightfield and fluorescence-detection optical module of xCELLigence eSight is an exemplary optical module that provides live cell imaging during impedance measurements, as described in U.S. Patent Application Publication No. 2021/0301245, incorporated herein by reference in its entirety for all purposes.

It will be appreciated that serial analysis may be performed by further instruments that take different measurements of the same sample, e.g., mass spectrometry, spectroscopy, phosphorescence lifetime imaging microscopy (PLIM) and/or fluorescence lifetime imaging microscopy (FLIM), including 2-photon excited imaging, and others.

Combination of Instruments

In certain embodiments, the cells may be analyzed in series by taking serial measurements of the same cell sample. In no particular order, the sample may be analyzed to measure bioenergetic work of the cell, such as the O₂, CO₂, pH. The data may be stored on a cloud-based storage and optionally analyzed on a cloud-based data processing and visualization system. The same cell sample, different samples, or samples from the same cell line may be analyzed using electrochemical measurements, e.g., impedance measurements. The data may be stored on the cloud-based system. The same sample, different samples, or samples from the same cell line may be visually observed for cell growth and morphology. The data may be stored on the cloud-based system. The data obtained from the independent measurements may be correlated with the corresponding samples/measurements by labelling the sample, e.g., by bar-code or other digital identification system. The data may be collected and collated in the cloud-based storage and optionally processed in the cloud-based data processing and visualization system. The collated data from analyzing the same cell sample may be interrogated for patterns and information.

Each of the measurements may be performed within the instrument described herein or in a combination of instruments, each operably connected to the data storage and processing system, e.g., cloud-based system or computer.

In certain embodiments, the sample may be analyzed in parallel by taking one or more aliquots of the original cell sample or samples from the same cell line to produce multiple substantially identical cell samples for each measurement to be taken, e.g., to produce three or more corresponding substantially identical samples. Each sample may be analyzed in a separate instrument, as previously described. The samples may be analyzed concurrently or substantially concurrently. The data may be collected and collated in the cloud-based storage system, as previously described. The collated data may be interrogated for patterns and information, as previously described.

In certain embodiments, the sample or aliquots of the sample may be analyzed to measure the bioenergetic work of the cell, by measuring parameters, such as the O₂, CO₂, pH, or other metabolically relevant parameters, and visually observed for cell growth and morphology at the same time, e.g., concurrently or substantially concurrently. In some embodiments, the sample or aliquots of the sample may be analyzed to measure the bioenergetic work of the cell, by measuring parameters, such as the O₂, CO₂, pH, or other metabolically relevant parameters, and for electrochemical, e.g., impedance, measurements at the same time, i.e., concurrently or substantially concurrently. In some embodiments, the sample or aliquots of the sample may be visually observed for cell growth and morphology and analyzed for electrochemical, e.g., impedance measurements at the same time, i.e., concurrently or substantially concurrently.

It should be noted that while the disclosure generally refers to measuring metabolism, similar methods may be used to measure or detect cellular microenvironment features, such as environmental conditions experienced by the sample. The conditions may be manipulated to drive towards a desired microenvironmental condition, possibly via environmental control. The conditions may be manipulated to relate to cellular response. As an exemplary embodiment, impedance, a specific imaged cellular parameter, a fluorometrically measured parameter (e.g., cellular metabolism), altering as a function of cellular oxygenation, oxygen or pH, e.g., may be controlled to effectuate a model to delineate the impact of tumor microenvironmental conditions on cellular function. As a further example, such properties may be controlled to analyze beat rate and/or metabolism of cardiomyocytes as a function of reduced oxygen and/or nutrient availability, with beat rate controlled either pharmacologically, or using electrical pacing via the device.

Embodiments described herein overcome the above disadvantages and other disadvantages not described above. Also, the embodiments are not required to overcome the disadvantages described above, and an example embodiment may not overcome any of the problems described above.

According to an aspect of an example embodiment, there is provided a device for analyzing one or more samples, the device including a support for a receptacle that holds a sample; an imaging subsystem that images the sample; and an analyzing subsystem that analyzes the sample.

According to an aspect of an example embodiment, there is provided a sample analysis method including selecting at least one subsystem from among a plurality of subsystems of a sample analysis device that examines one or more samples, the plurality of subsystems comprising an imaging subsystem that images the one or more samples and an analyzing subsystem that analyzes the one or more samples; and controlling the selected at least one subsystem to perform an examination on the one or more samples, the examination comprising an imaging operation of the imaging subsystem that images the one or more samples and an analyzing operation of the analyzing subsystem that analyzes the one or more samples.

According to an aspect of an example embodiment, there is provided a non-transitory computer-readable medium having embodied thereon a program which when executed by a computer causes the computer to execute a sample examination method, the method including selecting at least one subsystem from among a plurality of subsystems of a sample analysis device that examines one or more samples, the plurality of subsystems comprising an imaging subsystem that images the one or more samples and an analyzing subsystem that analyzes the one or more samples; and controlling the selected at least one subsystem to perform an examination on the one or more samples, the examination comprising an imaging operation of the imaging subsystem that images the one or more samples and an analyzing operation of the analyzing subsystem that analyzes the one or more samples.

According to an aspect of an example embodiment, there is provided a device for analyzing a sample. The device may include: a receptacle support configured to support a sample carrier (e.g., a microplate) comprising a sample carrier (e.g., a microplate) well configured to hold the sample, also referred to as a sample carrier, e.g., multi-well plate, plate, or sample carrier herein. In one embodiment, imaging of the sample is conducted using instruments such as automated cell imaging readers e.g., Cytation™ 5, Cytation™ 7, as disclosed in U.S. Pat. No. 10,072,982, incorporated herein by reference in its entirety for all purposes. In one embodiment, imaging of the sample is conducted using a confocal imaging device including: a receptacle support configured to support a sample carrier (e.g., a microplate) comprising a sample carrier (e.g., a microplate) well configured to hold the sample; an objective configured for imaging the sample; a laser point scanning confocal system configured to image the sample via the objective; and a spinning disk and/or wide field imaging system configured to image the sample via the objective, wherein at least a portion of both the laser point scanning confocal system and the spinning disk and/or wide field imaging system is movably provided such that the laser point scanning confocal system and the spinning disk and/or wide field imaging system are configured to be selectively aligned with the objective for imaging the sample.

It will be appreciated that the cell sample may be observed using any type of imaging modality that can visually examine the cells.

In certain embodiments, the cell sample may be observed using phosphorescence lifetime imaging microscopy (PLIM) and/or fluorescence lifetime imaging microscopy (FLIM), including 2-photon excited imaging.

In certain embodiments, an imaging modality known as confocal imaging may be well-suited for imaging the cell samples, e.g., 3D cell structures such as spheroids. In confocal imaging, a sample may be illuminated one point or portion at a time. For example, light may be passed through a small aperture such as a pinhole positioned at an optically conjugate plane. The point illumination substantially eliminates out of focus light and background light, and thereby increases the optical resolution and contrast of the image. The complete image, built or stitched together point by point via a scanning function, is very sharp with well-defined features. The scanning function may be performed with the spinning disk, also known as scanning disk or Nipkow disk.

Confocal imaging is a particularly well-suited imaging modality to be used with spheroids. With confocal imaging, a spheroid can be sectioned, layer by layer, and a 3D model may be created in a computer for both exact cell counting and 3D image manipulation to observe a spheroid from various angles.

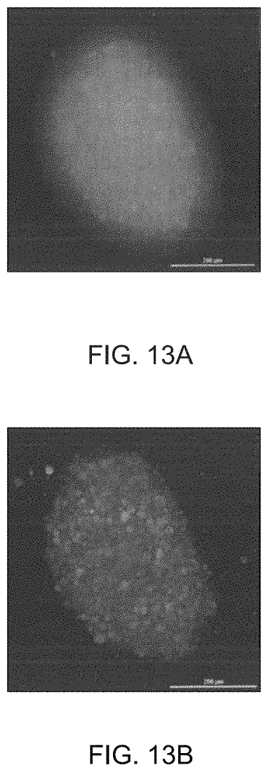

FIG. 13A-13B are a comparative illustration of a spheroid. FIG. 13A illustrates a spheroid taken at twenty times (20×) magnification with wide field imaging. FIG. 13B illustrates the spheroid taken at twenty times (20×) magnification with and confocal imaging. While the size of the spheroid may be assessed using the image of FIG. 13A, the individual cells and spheroid structure only become visible with the confocal imaging in FIG. 13B.

The advantage of resolution attributed to confocal imaging of FIG. 13B is provided at the expense of decreased light intensity caused by confocal aperture, such that longer exposure times are often required in comparison to wide-field imaging of FIG. 13A.

The addition of confocal fluorescence imaging to an instrument that also includes non-imaging analysis modalities (fluorescence, absorbance, chemiluminescence, etc.) and wide-field fluorescence imaging on a cell level combined with a controlled live cell environment would deliver to a modern researcher the most versatile single instrument for analyzing sample carrier (e.g., microplate)-based assay formats, including those aimed at 3D cell spheroids research.

In an example, there may be a workflow in which wide-field imaging is performed for faster screening, while confocal imaging is performed for publication images related to the O₂, CO₂, pH measurements obtained from sample.

Wide-field imaging may be performed for an HCS type assay, in which the throughput is quicker with wide-field imaging, and the resulting image analysis is still statistically robust. Then, confocal imaging may be employed to acquire representative wells of the “hits” compared to “controls” for publication or presentation purposes.

In an example, there may be a workflow in which wide-field imaging is performed for a quicker primary screening of spheroids based on size. Then, confocal imaging is used for deeper assessment of the size of each “hit” wells, based on nuclear count, which is more accurate using confocal imaging.

Typically, wide-field imaging cannot “see” into the 3D spheroid well enough to reliably count individual nuclei, however, wide-field could still make determinations of “hits” based on total spheroid size. Once “hit” wells are identified with wide-field imaging, identified wells could then be imaged with confocal imaging, to obtain improved image analysis for counting total nuclei in the spheroid, which wide-field imaging alone could not perform.

In an example, there may be a proliferation Assay (3D Endothelial Cell Spheroid Assay) to determine wound healing drug/compound/substance candidates. A primary drug/compound/substance screen may be performed in sample carriers (e.g., microplates), in which small endothelial spheroids are treated with an unknown compound library to determine which compounds elicit increased cell growth/proliferation. Compounds that cause increased growth may be contenders for further wound healing studies.

In an analysis workflow, a plate reader may be used to quickly screen the sample carrier (e.g., the microplate) using GFP fluorescence intensity, to determine wells with spheroids of increased size. Wells that meet a threshold of GFP intensity (threshold is statistically determined during assay development) are considered “hits” and selected to be further imaged. Control wells are also always imaged further, as reference wells for comparison with hit wells. Confocal imaging of 3D spheroids may be performed to acquire two-channel z-stack image set (Hoescht 33342 Nuclear marker and GFP marker) of the entire spheroid sample. In image processing and analysis of a maximum projection of Z-stack, a cellular count of spheroid is determined to quantify spheroid size. Visual inspection of distribution of nuclear masks in the image, to determine if there is cell death within the spheroid, is performed. And, results from hit well image analysis are compared to the controls to determine percentage growth against controls.

In an example workflow, 3D tumoroid cytotoxicity and immune response assay (3D Tumoroid Assay from surgical samples to determine Immune and cytotoxic therapeutic response) is performed. The assay involves culturing tumoroids obtained from surgical samples derived from animal models or patients. Because these tumoroids are derived from animals/patients, in-vitro tumor-derived immune cells responses can be evaluated, enabling analysis of tumor response to various therapies. This assay can assess the effectiveness of novel therapeutics in sample carrier (e.g., microplate)-based format using a heterogeneous multicellular tumor model.

For example, tumoroids may be stained for nuclear count (e.g., blue) and stained for immune cell marker (e.g., red). A sample carrier (e.g., microplate) reader may be used to assess: wells with high cytotoxicity shown as low blue signal; wells with high immune response shown as high red signal. Wells that meet one or both threshold criteria for cytotoxicity or immune response (threshold is statistically determined during assay development) are considered “hits” and selected to be further imaged. Control Wells are also always imaged further, in order to compare to hit wells. Confocal imaging of 3D tumoroids is performed to acquire two-channel z-stack image set (Hoescht 33342 Nuclear market and CY5 marker) of the entire tumoroid sample. Image processing and analysis is performed for the maximum projection of Z-stack, and cellular count of tumoroid is performed to quantify cellular count. Count of red positive cells is determined for the immune response. Results from hit well image analysis is compared to the controls to determine percentage cytotoxicity or immune response against controls.

Several of the above examples utilize the ability of a single instrument to run an assay as “hit picking.” The first rapid read identifies the samples of particular interest, typically using a fast reading method that can be fluorescence non-imaging reading or fluorescence or bright field wide-field imaging reading performed at lower magnification. Once wells of interest are identified, called hits, a second more time consuming modality is deployed to determine results of particular interest. This processing is of particular importance if final results are high resolution confocal imaging, in which large data storage is required and gathering vast amount of information on only a few samples that are of interest provide substantial savings of the data storage space. This processing also saves a processing time during data acquisition and data review, as most samples are not “hits” and are dismissed during the first assay step. A single unified device to perform the various disparate processing steps can streamline the analysis.

Other applications of the capabilities of the single instrument with the diverse functionality to study of spheroids are possible. Spheroids are typically grown in round bottom wells. Often, for the final imaging step, spheroids are transferred into flat bottom plates for the purpose of preventing the rounded well bottom as functioning similar to a lens during imaging, thereby unnecessarily inducing optical aberrations and negatively affecting the resultant image quality. High quality microscope objectives are not designed for such “roundwell” bottom lens in the optical path. After transfer into another well, dish, or plate for the best image quality, the exact location of the spheroid in the well is no longer known. In a preferred embodiment, wide-field imaging at lower magnification but larger field of view to image the well could be performed to locate the spheroid (region of interest), then position the well to bring the found spheroid location (region of interest) in line with the optical axis and use a higher magnification objective with smaller field of view to image the spheroid in confocal modality and perform Z-stack, by collecting multiple images while the objective traverses along the objective's focusing axis, perpendicular to the well bottom surface. The spheroid (region of interest) may be identified by using a non-imaging analysis modality of the instrument by performing fluorescence read area scan and selecting the region of a maximum fluorescence signal fur imaging.

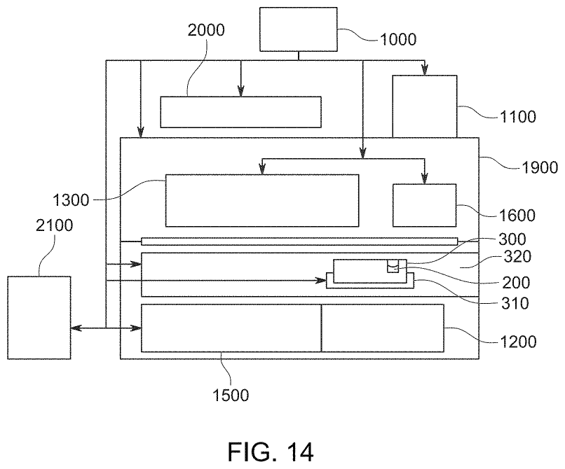

FIG. 14 is a block diagram illustrating a multi-detection system according to an embodiment.

As illustrated in FIG. 14, the multi-detection system includes a controller 1000, a fluid injection subsystem 1100, an imaging subsystem, including wide-field imaging components 1200 and confocal imaging components 1500, a non-imaging analysis subsystem 1300, an imaging illumination subsystem 1600 for wide-field imaging, housing 1900, a sample carrier (e.g., microplate) 300, a carrier for a sample carrier (e.g., a microplate carrier) 310, incubation chamber 320 for incubating a sample in a well 200, an environmental control subsystem 2000, and a confocal imaging subsystem. The multi-detection system may also include an external subsystem 2100.

Samples are placed into wells 200 (e.g. microwells) of the sample carrier (e.g., the microplate) 300. The sample carrier (e.g., the microplate) 300 is transported by the sample carrier (e.g., the microplate) carrier 310 into and out of the measurement and incubation chamber 320. When disposed to be exposed to an external environment of the multi-detection system, the sample carrier (e.g., the microplate) 300 may be accessible outside the incubation chamber 320 and/or housing 1900 for access by a technician or robotics arm. When the sample carrier (e.g., the microplate) 300 is disposed within the chamber, various supported imaging and non-imaging analytical modalities may be performed.

The sample carrier (e.g., the microplate) carrier 310 is part of a sample carrier (e.g., a microplate) transport subsystem for positional manipulation of the sample carrier (e.g., the microplate) 300, and may include any suitable combination of belts, platforms, sample carrier (e.g., microplate) holders, motors, and positioning software executed under hardware control for the positional manipulation. When the sample carrier (e.g., the microplate) 300 is disposed within the incubation chamber 320, the entire sample carrier (e.g., microplate) 300 remains incubated. The incubation system and incubation chamber 320 will be later described in detail.

The non-imaging analysis subsystem 1300 may be based on illumination via a flash bulb, dual excitation monochromators, and dual emission monochromators, photomultiplier tubes (PMT), and silicon detectors. The non-imaging analysis subsystem 1300 supports absorbance, fluorescence, and chemiluminescence analysis modalities for detection of corresponding properties of the sample in the well 200. The non-imaging analysis subsystem 1300 may be implemented as a filter-based subsystem or as hybrid of any or all of the above.

The imaging subsystem includes wide-field imaging components 1200 and confocal imaging components 1500, such as objectives, lenses, LEDs, filter cubes, spinning disks, cameras and other components. The imaging illumination subsystem 1600 includes illumination components for wide-field imaging and is able to provide illumination for bright field, color bright field, and phase contrast imaging modalities.

The external subsystem 2100 may be an external confocal illumination subsystem for confocal imaging that can be modularly connected to and disconnected from the imaging subsystem within the housing 1900 via fiber optics for added flexibility of the physical placement of the external subsystem 2100 relative to the instrument. Alternatively, the confocal imaging illumination subsystem may be disposed to be integrated within the housing 1900.

The fluid injection subsystem 1100 delivers reagent to the wells 200, if required by an assay. The fluid injection subsystem 1100 may include any combination of pumps, reservoirs, lines or tubing, pipettes and tips, and software executed under hardware control for delivering, and if necessary aspirating, fluid to and from the wells.

The environmental control subsystem 2000 shown externally placed relative to housing 1900 may include a gas control module that provides control of atmospheric conditions inside the housing 1900. Other control modules may include modules for control of temperature, humidity, and other conditions, which may be controlled within the housing 1900 under control of the environmental control subsystem 2000. The environmental control subsystem may include any combination of pumps, reservoirs, lines or tubing, fans, heating and cooling elements, and the like for controlling all conditions within the housing 1900. The housing 1900 houses most of the subsystems and defines the physical space in which gas atmosphere, conducive to live cells, can be effectively maintained and controlled by the environmental control subsystem 2000.

The controller 1000 may control all operations of the multi-detection system. The controller 1000 may communicate by wire or wirelessly to each of the various subsystems in the multi-detection subsystem. The controller 1000 may include any combination of hardware (e.g., CPU, memory, cables, connectors, etc.) and software for execution by the hardware for controlling operations of the multi-detection system.

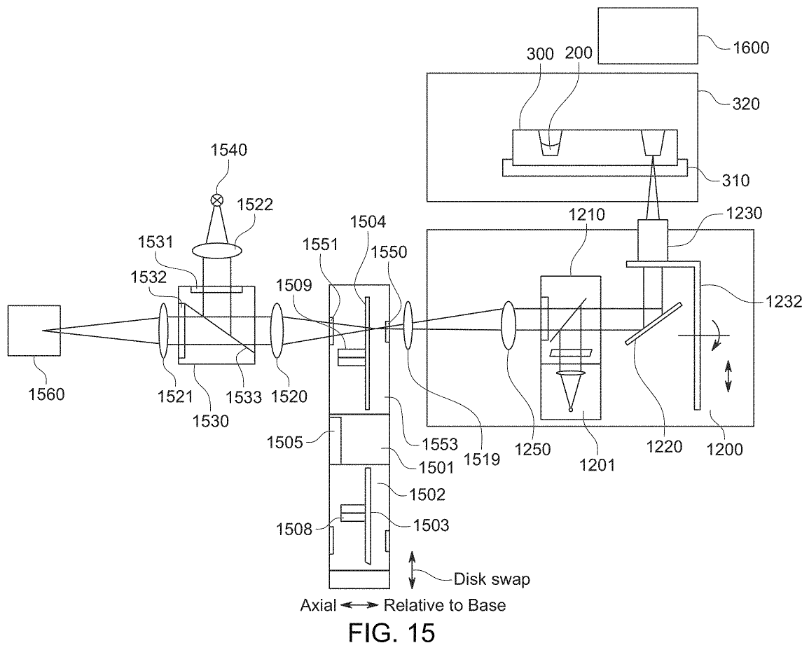

FIG. 15 is a block diagram illustrating a multi-detection system according to an embodiment.

Several imaging modalities are made possible by the multi-detection system. Wide-field imaging in fluorescence, bright field, and phase contrast may be performed in additional to the confocal imaging modality. Optical elements of both the confocal imaging system and wide-field imaging systems are shown in FIG. 15.

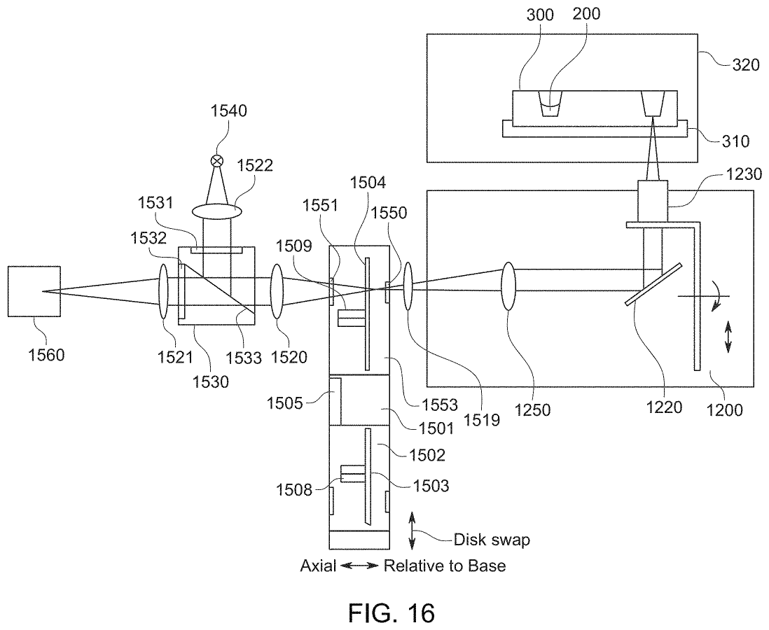

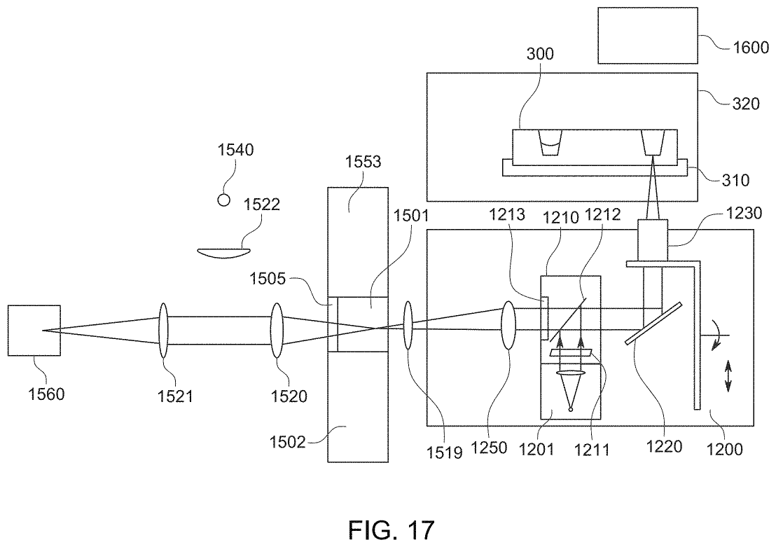

A sample carrier (e.g., a microplate) 300 may be placed onto a carrier for a sample carrier (e.g., a microplate carrier) 310 that positions the well 200 of interest in line with an imaging optical axis of the objectives 1230. An objective may be selected from among several objectives of various magnifications placed on an objective turret 1232. The relative position of the imaging illumination subsystem 1600 is illustrated in FIG. 15, and the imaging illumination subsystem 1600 may be used for bright field, color bright field, and phase contrast imaging to the sample. Many optical elements are shared between wide-field and confocal systems and more detailed description of such sections will be provided below in FIGS. 16-17, in which some elements of FIG. 15 are omitted for clarity.

FIG. 16 is a block diagram illustrating a multi-detection system according to an embodiment.

Confocal imaging as deployed as shown in FIG. 16. Wide-field imaging subsystem elements (e.g. LED cube 1201 and filter cube 1210) are automatically removed from the optical path to the sample and the system shown in FIG. 15 is transformed into the confocal optical system illustrated in FIG. 16, for understanding of the confocal light path.

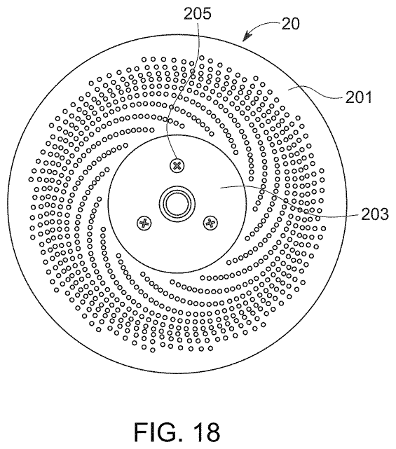

A spinning disk confocal system is deployed as an example embodiment of the confocal imaging system. The system is based on utilizing a spinning disk (FIG. 18) the optical path. The disk is placed in the intermediate image plane conjugal to a sample and detection planes. The disk is thus both in the excitation light path and the emission light path. The disk is typically around 2 mm thick and made from glass or quartz, in an example embodiment. The disk may be coated to be non-transparent, or having a given transparency or opacity, except for clear areas left as a pattern of pin holes or slits. Ideally the disk surface is made to not reflect oncoming light. A sample to be imaged is illuminated by excitation light transmitted via the pin holes. Only radiation emitted by the sample, which is generated from these illuminated spots on the sample, reaches a detector via pin holes of the disk. The pin holes or slits, while many, are spaced far away from each other to act optically independently. The energy from adjacent pin holes does not ideally affect the sample spots illuminated by a given pin hole. The disk spot pattern is typically arranged in several spirals as shown in FIG. 18. The disk may be controlled to continuously spin, thus scanning the sample. As the disk rotates, the sample is illuminated one spot at time and the complete sample image is detected on the detector for reconstruction as a complete image of the sample.

Returning to FIG. 16, the confocal light source 1540 may be any light source suitable for confocal microscopy. For example the confocal light source 1540 may be a solid state light source, such as a light emitting diode (LED) or solid state laser or semiconductor-based laser (laser diode). In an example embodiment, the output tip of the optical fiber may be a light (radiation) source. Radiation is as an embodiment, as the excitation spectrum could be outside of 380-630 nm range that is commonly referred as light. However, the term “light source” is more commonly used in imaging, and the term light will be used interchangeably with radiation herein. The input tip of the fiber can be illuminated from a light source module external to the instrument to allow flexibility in selecting the best light source match for the sample imaging needs. The fiber also allows flexibility of bifurcating input from multiple external light sources. The output tip of the fiber is imaged by condenser 1522 onto or close to the intermediate sample image plane where spinning disk 1504 is located. The light from the fiber may be sent through excitation filter 1531 and then is reflected from the dichroic mirror 1533 and focused by the tube lens 1520 onto the spinning disk 1504. The term “lens” here and throughout the description may refer to a single lens or group of lenses depending on the embodiment and function, as appreciated by person skilled in the art. As discussed, the disk has a spiral pattern of holes of slits. A field lens 1519 minimizes the light loss and guides the light exiting the disk to be gathered by the tube lens 1250. The tube lens 1250 guides the excitation radiation into objective 1230 via mirror 1220. The objective 1230 illuminates the small spots on the sample near the bottom of well. The sample components have been stained with dye that corresponds to excitation wavelength. Those components are excited with oncoming radiation and emit radiation that typically has a longer wavelength. This emitted light is guided to the detector as follows.

Light emitted by a sample is collimated by objective 1230, and is reflected by mirror 1220 and gathered by tube lens 1250 and field lens 1519 onto spinning disk 1504. The intermediate image of the sample in emitted light is formed at the spinning disk 1504 surface. The tube lens 1520 and lens 1521 invert that image and form a sample image at the detector 1560. The detector 1560 is typically a pixilated digital camera, such as charged couple device (CCD) camera or complimentary metal-oxide semiconductor (CMOS) camera. The sample image is captured by the camera, and may be stored in memory of the multi-detection system or an external computing system, and could be enhanced and analyzed for various properties and/or presented to the user on a visual display.

A confocal cube 1530 (e.g., a confocal excitation/dichroic mirror/emission cube) is shown between the tube lens 1520 and lens 1521, which is an arrangement for fluorescence microscopy. The filters and dichroics may be thin film coatings on glass. Excitation filter 1531 forms a bandpass for excitation and emission filter 1532 forms a bandpass for emission, while the dichroic mirror 1533 separates excitation and emission to fully use the available energy and to suppress magnitude of excitation light reflected from multiple optical surfaces as excitation light travels towards the sample, including the disk surface, that reaches the detector. The lens 1521 (e.g. an emission filter) provides most of the excitation light suppression, but the dichroic mirror 1533 also plays a suppression role. An alternative arrangement for the described cube could be several filter wheels that carry excitation filters, emission filters and dichroics. In the exampled embodiment, cubes are a method of arranging the described elements, which allows very easy exchange by a user as imaging needs change. Several filter cubes (e.g. confocal cubes 1530) can be arranged on a motorized slider and could be identified either by setup in software performed by user or labelled electronically or optically with a code to be read automatically via bar code or some other automatic available method.

The surface of the spinning disk is imaged onto detector along with the sample. Thus, any dust particles that attach to the disk surface may show up as artifacts in the image, for example streaks of bright light due to disk rotation. The small particles can easily adhere to the disk surface with sufficient force that resists centrifugal forces. The spinning disk 1504 and the disk drive motor 1509 are part of a disk module 1553. The disk in the module is typically assembled in clean environment, like clean room, and is sealed from the ambient environment to prevent dust particles from settling on the disk. The windows 1551 and 1550 in the module allow light to pass through, but keep dust out. Ideally, these dust protection windows should be placed as far as feasible from the intermediate image plane so dust that could settle on the window glass does not result in artifacts in the image. The disks are fully contained within the disk modules 1502 and 1553. Thus, the user should not open the modules to avoid introducing particles of dust to the disk.

FIG. 16 illustrates two disk modules 1553 and 1502 installed in the multi-detection instrument. The disks can be moved to position one disk or another disk into the optical path. Alternatively, both disks can be moved out of the light path and space 1501 placed along the optical axis. This allows for wide-field imaging modality to be performed, such as fluorescence imaging, bright field imaging, or phase contrast imaging.

A great benefit of allowing both confocal and wide-field imaging options for the user in the same instrument is ability to overlay images in various imaging modalities, such as a wide-field image and the same image in confocal imaging modality, for example. Alternatively, a bright field image may be utilized to locate a region of interest that is then imaged confocally. For this arrangement to properly obtain an image, the magnification in both modalities should match exactly or the images do not overlay properly. The light in the section between the tube lenses 1520 and 1250 is not parallel. In confocal modality, several flat windows are present in the optical path in this section: confocal disk and dust protection windows. There is no need for these windows in the wide-field modality. But, to match optical path length in the non-parallel light path, the glass 1505 is added in the space 1501 between confocal disks through which wide-field imaging takes place. This assures that a sample remains in focus for a fixed objective position when the image modality changes. This assures that magnification in confocal and wide field imaging modes match. The thickness of glass 1505 should match the sum of flat windows of a disk used in confocal imaging (window 1551, spinning disk 1504, and window 1550). The glass 1505 should be placed as far as feasible from the intermediate image plane so dust that could settle on the glass does not result in artifacts in the image.

The pin hole size on the confocal disk is ideally selected based on the parameters of an imaging objective 1230. In an embodiment, the size of image of the disk pin hole made on the sample may be matched to the distance between the first two minima of the Airy diffraction pattern of objective. The formula for Disk pin hole size, as given in Zeiss “Introduction to Spinning disk microscopy,” is

Disk pin hole diameter=1.2*Magnification of objective*Emission Wavelength/Numerical Aperture of Objective.

Both numerical aperture (NA) of the objective and magnification are part of the formula. If a pin hole is too small, too much light is lost and time to take an image increases. If a pin hole is too large, the confocal effect can be reduced or lost altogether. Most commercial spinning disk microscopes feature non interchangeable spinning disk with pin holes in range 50-70 um. This works reasonably well as a compromise with the range of high magnification objectives typically deployed with confocal microscopy. But it is preferred, a disk with appropriate pin holes can be matched to the objective used.

Some spinning disk implementations do not possess a spiral pattern of round holes, but instead employ slit apertures. Slit apertures may provide a relatively brighter illumination of the sample and more intense emission signal, whereas pin hole apertures may provide relatively better axial resolution. Hence, for some imaging applications, including biological fluorescence application slits may be preferred to be able to reduce image acquisition times, which is another reason to change the disk even for a fixed objective.

Multiple disks may be deployed in the imaging instrument so that selection from among the disks may be performed by the user or automatically by the multi-detection system.

FIG. 16 illustrates an example of two disk modules 1502, 1553 used in the multi-detection instrument. All disk modules can be configured to be replaced by the user. The modules can be identified either by setup in software controlled by user or labelled electronically or optically with codes to be read automatically via bar code or some other available method, to enable automatic configuration by the multi-detection system.

One additional advantage from a modular disk module is the ability for the user to clean the windows 1551 and 1550, which may provide dust protection, when the disk module is removed from the instrument and both windows are easily accessible.

Module identification enables automated software setup and to automatically reset and calibrate the module axial position in the optical path. In the spinning disk confocal imager the disk surface plane, detector sensitive element plane and sample planes should be conjugate to each other. This means, if following emission rays from sample, the image of sample plane is coincident with the disk plane, and disk planes and sample planes images are coincident with the detector plane. The detector 1560 sensitive chip plane is fixed by camera design. The objective 1230 can be moved along the focusing axis to sharpen the sample image on the detector. Then, the disk should be ideally placed in the intermediate plane conjugal with both the detector and intermediate sample image plane for all three planes to be conjugate. In a proposed embodiment, a disk axial position is held very close to an ideal conjugate position by disk module design, but the final position of the disk surface can be adjusted automatically by observing the disk pattern on the detector and bringing this pattern into sharp focus on the detector. Multiple image based focusing methods are available and are well known in the industry. Once a best disk surface position is found, this position can be stored in software and memory, and associated with the disk module. If the disk module is removed and reinstalled, the correct disk position can be restored automatically by software. If a new disk module is introduced, the system will alternatively engage the disk focusing routine and will select the best axial position for the new disk module. The user thus can be relieved from keeping track of what disk module is deployed in the instrument, and the various positioning thereof.

Alternatively, if only a few disk modules are envisioned to be utilized, then a user can setup disk modules via a setup screen in the calibration section of a user interface of software included with the multi-detection system.

The two concepts of user replaceable disk module and automated axial disk positioning work best in tandem, but may be separately implemented. If automated axial disk positioning is unavailable, the disk modules may be configured to be interchangeable relative to the disk position and some datum on the module that assures proper placement in the instrument. The concept of easily replaceable disk modules, that user does not have to open and thus subject to environment, would still apply and bring benefit to the user who wants flexibility of multiple disks best suited for deployed imaging objectives and samples.

Even if disk modules are limited to one or two in the instrument, the automatic axial adjustment can be used to alleviate the need to strictly control location of the detector image sensor sensitive surface in the detector 1560 (e.g. camera). In the case to allow user maximum flexibility in camera selection and to also allow upgrade of camera within the multi-detection s system. If the sensor surface after camera replacement moved, the disk surface can be relocated automatically to be conjugate to sensor surface via image-based autofocus routine.

FIG. 17 is a block diagram illustrating a multi-detection system according to an embodiment.

In FIG. 17, wide-field imaging as deployed in an example embodiment is illustrated. As described above, the optical section (with elements labelled 15xx) does allow both confocal imaging (with spinning disks 1504 or 1503 in optical path) and wide-field imaging (via space 1501 between the disks). But, there may be a shortcoming of using this optics and confocal light source 1540 and confocal cubes 1530 for wide-field modality the researcher may want to deploy in a single versatile instrument. For confocal imaging, the excitation radiation should be directed onto the disk via multiple optical elements (e.g. dichroic mirror 1533, tube lens 1520, window 1551) positioned prior to the disk surface. After the disk, excitation radiation is guided to the sample via more optical elements (e.g. window 1550, field lens 1519, tube lens 1250, mirror 1220, objective 1230). For confocal imaging, there is no choice to this scheme. But, on every surface encountered, some of excitation light is reflected back. Good design then relies on careful ray tracing to ensure that reflected light is kept from the detector as much as possible and on the emission filter 1532 to suppress the unwanted reflected light. The optical elements prior to the disk surface. as tube lens 1520 and window 1551. and the spinning disk 1504 surface are exposed to very strong level of excitation radiation that partially gets reflected. Also, any dust particles may get excited and will fluoresce. Despite the best intention of the designer, some of the light does come through to the detector and reduces signal to noise ratio. Thus, a non-fluorescing sample that should appear very dark on the image, may not appear very dark. This may be due to noticeable background signal due to reflected light, the effect that tends to be uniform across the image. For wide-field microscopy using the confocal section excitation elements described above in FIG. 16 would come with significant compromise in image quality and system capabilities.

In an example embodiment, an alternative subsystem is provided in the same instrument that can be used for wide-filed fluorescence imaging. Confocal cubes 1530 of a confocal subsystem are positioned out the way and spinning disk module gets positioned to the space 1501 for wide-field imaging. This transforms the configuration of FIG. 15 into the configuration of FIG. 17. The dedicated wide-field section elements are an LED cube 1201, and wide-field excitation/emission/dichroic imaging filter cube 1210. The excitation filter 1211, dichroic mirror 1212 and emission filter 1213 are mounted in a filter cube that typically will be matched with the LED cube 1201 for best signal to noise performance. Several of these cube pairs, corresponding to specific chemistry being investigated, can be provided on a slider.

There are several advantages to this design.

First, is that the LED excitation optics is much nearer to the sample, and thus excitation light encounters fewer optical surfaces on the way to sample. Reflections from those surfaces, that can reach the detector, are thus greatly reduced, and signal to noise in the image is improved.

Second, is the wide verity of LEDs used in LED cubes 1201 that are available in the market that may not be powerful enough to be used in the confocal optical tract, but can deliver sufficient excitation if placed closer to the sample as shown in FIG. 17.

Third, particularly important if sample has to be excited in UV range, is that some objectives are rated as UV objectives and transmit UV light and exhibit very low fluorescence when excited by UV. But, in general optical elements commercially available for the rest of optical tract, such as tube lenses, are not assured to be fluorescence free when illuminated by UV light. If a wide-field image of a sample stained with common DAPI nuclear stain is required, a common approach in the confocal optical tract is to use wavelength around 400 nm, and thus to avoid strongly exciting optical elements in addition to the sample. But moving excitation towards 400 nm from 360 nm, the wavelength that is ideal for DAPI stain excitation, reduces emitted light a great deal. A researcher would need to place higher concentration of dye in the sample or raise the detector gain, and thus reduce signal to noise of imaging. Ideally the excitation of DAPI stained sample will be done at 360 nm, but the UV excitation light will not pass through optical elements that may fluoresce. LED Cube 1201 and filter cube 1210 allow just such an optimum option in an example embodiment. The UV excitation enters only objective 1230 that can be selected to not fluoresce. The emitted light does pass back to detector via multiple optical elements common to confocal and wide field tract, but because emitted light is in the visible spectrum range, the optical elements the light encounter do not typically fluoresce at the level they do in UV light.

FIG. 17 shows a relative location of an imaging illumination subsystem 1600 for wide field imaging in non-fluorescing modalities. This can be bright field, color bright field with tri color LEDs switchable one at a time, or phase contrast illumination system with ring apertures that would be matched to phase contrast objectives.

Additional embodiments and components of imaging systems are further described in, PCT Patent Application Publication No. WO2022120047A1 “Universal multi-detection system for microplates with confocal imaging,” which is incorporated herein by reference in its entirety for all purposes. Such components include, e.g., a laser point scanning confocal (LSC) modalitya laser point scanning confocal (LSC) system, a spinning disk confocal system, and wide field functionality in a single device, etc.

Components of the widefield imaging system are further described in, e.g., U.S. Pat. No. 10,072,982 titled “Universal multidetection system for microplates”, which is incorporated herein by reference in its entirety for all purposes.

A configuration according to embodiments of the present disclosure may also incorporate or include a laser point scanning confocal system, a spinning disk confocal system, and wide field functionality in a single device is described below. However, embodiments of the present disclosure may include any combinations of the above systems and functions.

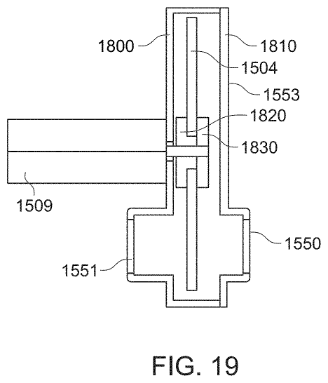

FIG. 19 is a diagram illustrating a confocal disk imaging module according to an embodiment.

A disk drive motor 1509, a DC brushless motor in an example embodiment, capable of high rotational speed of several thousand RPM at a constant velocity, is mounted to the housing base 1800. The spinning disk 1504 is secured on the motor shaft by the hub parts 1820 and 1830. The cover 1810 mounts to the housing base 1800 to complete a dust free environment for the disk. There is no user access to the disk. Optical windows 1550 and 1551 allow light to pass therethrough while keeping an interior of the module dust free. It is advantageous from imaging standpoint to keep both windows as far away from disk plane as feasible, within overall space constrains, to avoid dust particles on the windows affecting the image. The disk module can be identified via bar code label, simple binary code label or some other instrument readable means so the multi-detection system can automatically identify which disk modules are present and available at any one time.

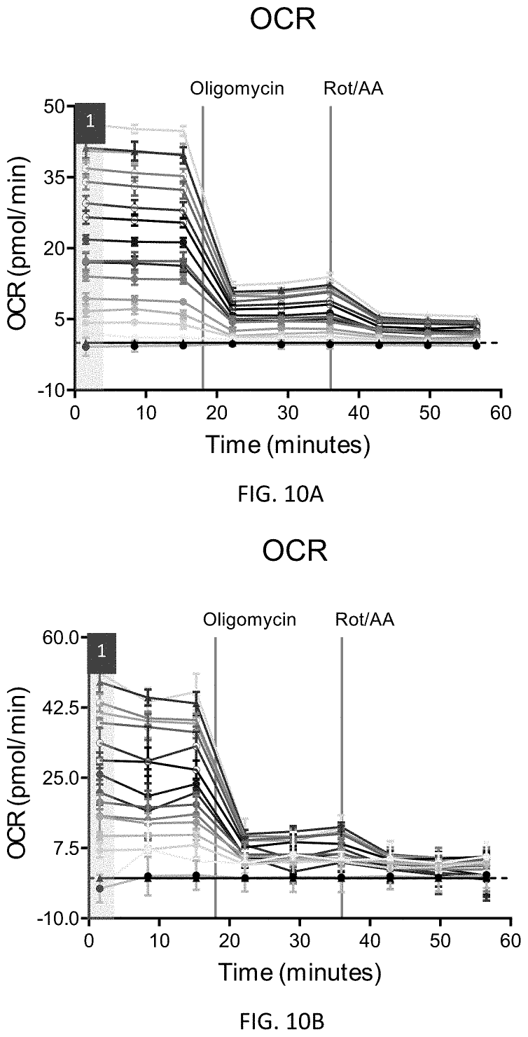

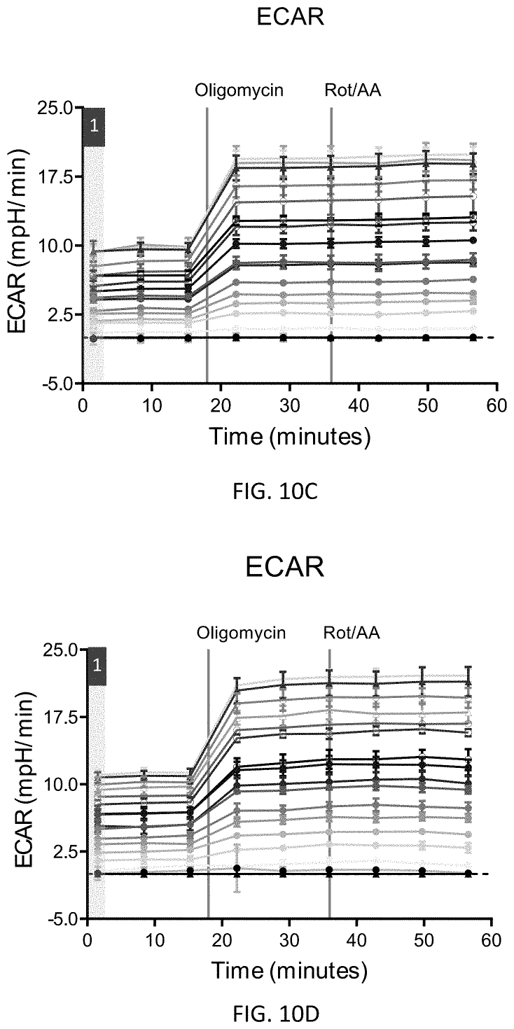

Referring to FIG. 18, there is a need to closely correlate the disk speed and confocal image exposure time. Multiple spirals are provided on the disk as seen in FIG. 10 and, as the disk rotates, the sample is swept by the pin hole pattern. There is a minimum angle of disk rotation required to sequentially, but completely, illuminate the sample once. For many commercial disks, and the disk of an example embodiment, this angle is 30 degrees. If the exposure time is not a multiple of times to move the disk 30 degrees, some artifacts like stripes becomes apparent in the image. This is well known problem in the industry. In an example embodiment the speed of disk rotation is set at 2400 rpm and the exposure time is set in multiples of one full revolution (e.g., 25 msec, 50 msec, 75 msec, etc.). This approach was found to result in a good compromise between image quality and minimum time to take an image. Also this approach, of using full revolution time exposure increments, resulted in minimizing image artifacts caused by potential non-concentricity between the disk spiral pattern and the disk rotational axis.

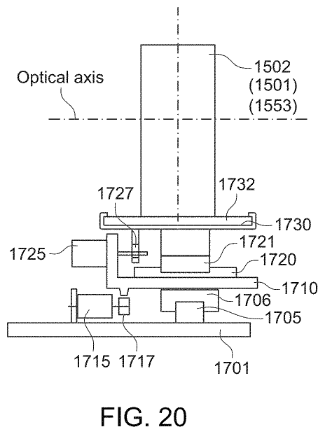

FIG. 20 illustrates a disk changing mechanism and a disk focus mechanism according to an embodiment.

Referring to FIG. 20, the disk changing mechanism and disk focus mechanism may be implemented in an example embodiment. However, the configuration of the disk changing mechanism and the disk focusing mechanism are not limited thereto.

The base 1701 supports all elements of the mechanism. A linear way rail 1705, like part of an IKO or HTK guide system, is attached to the base 1701. The carriage 1706 of a linear way supports a bracket 1710. The bracket 1710 is translated by motor 1715 via timing belt 1717 in the direction perpendicular to the optical axis. The motion allows for either disk module 1502 or disk module 1553 or space 1501 to be positioned in alignment with the imaging optical axis. Other mechanical implementations are possible, the main advantage of the timing belt is the speed of change that is achievable with this particular method. The axis homing sensors and/or possible encodes are not illustrated for clarity.

The bracket 1710 in turn carries linear way rail 1720 and motor 1725. In an example embodiment, the motor shaft is shaped as a lead screw. The motor via lead nut 1727 translates the support 1730, attached to linear way carriage 1721 in a direction of optical axis to provide axial focus for the confocal disks. The axis homing sensors and/or possible encodes are not illustrated for clarity.

The disk modules can be attached to the support 1730 directly and accessed by user. The attachment could be via fasteners or via magnets for easy removal. Alternatively, disk modules could be attached to the guide 1732, which in turn could be slip fit and secured into support 1730 for easy removal from the instrument by user.

Other mechanisms can be deployed to accomplish the function of disk module access, positioning and disk focusing as will be understood by person familiar with the art.

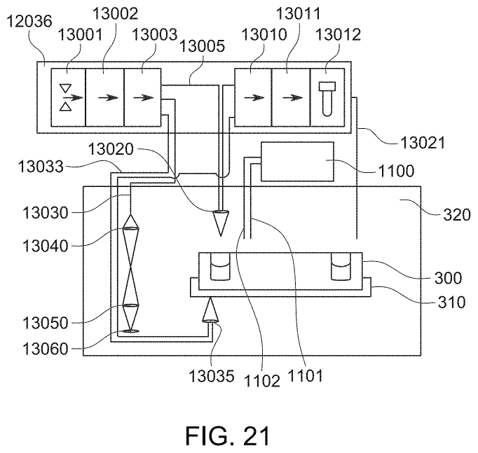

FIG. 21 is a diagram of a non-imaging analyzing subsystem according to an embodiment.

Referring to FIG. 21, the non-imaging analysis subsystem 1300 of the multi-detection system is provided.

The analytical modalities of the non-imaging analysis subsystem 1300 may be absorbance, fluorescence from top and bottom, and chemiluminescence. The Xe flash bulb 13001 emits radiation in the range 200-1000 nm. The two stages 13002 and 13003 of fluorescence excitation/absorbance dual monochromator select a narrow band pass of radiation. The radiation is guided towards sample by fiber optics cables to either absorbance channel via fiber 13030, top fluorescence via 13005 or bottom fluorescence via 13033. Only one fiber is acting at a time so there is no cross talk of light among various analytical modes. Absorbance is measured via lenses 13040 and 13050 by silicon detector 13060.

Top fluorescence excitation and emission pick up are performed via lens 13020, which can move up and down to accommodate various sample carrier (e.g., microplate) and fluid levels. Bottom fluorescence is done in similar manner with lens 13055. Both top and bottom emissions are guided by fiber optics cables to the first stage of the emission dual monochromator 13010 and 13011 and then to photomultiplier 13012. The chemiluminescence fiber 13021 can be connected directly to the photomultiplier to offer measurements for very faint light via bypassing monochromator.

The fluid injection subsystem 1100 can provide researcher with ability to inject reagent via fluid lines 1112 and 1111 and rapidly measure results of injection by analysis subsystem further increasing range of test that can be performed in the instrument.

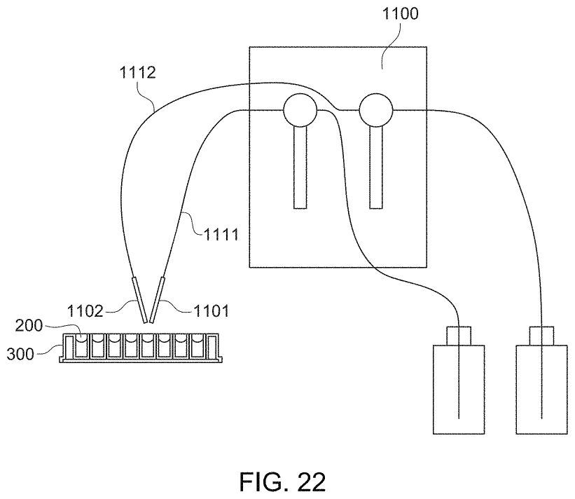

FIG. 22 is a diagram illustrating an injection subsystem according to an embodiment.

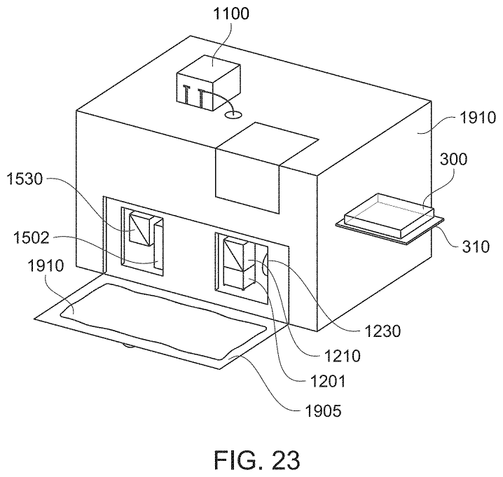

Referring to FIG. 22, an optional injection subsystem is provided. The injection subsystem 1100 can be placed on top of the multi-detection instrument, and fluid lines 1112 and 1111 fed through the bulkhead access in the top of the housing, as shown in FIG. 23. The reagents are delivered to microwells by pumps in the fluid injection subsystem 1100 via fluid lines 1111 and 1112 that can be PTFE lines, and into wells via injection needles 1102 and 1101, as shown in FIG. 22.

Referring to FIG. 21, environmental control may deployed in the multi-detection system.

The carrier for the sample carrier (e.g., the microplate carrier) 310 supports the sample carrier (e.g., the microplate) 300 and is located in the incubation chamber 320, as shown in FIG. 21. This assures that sample carrier (e.g., microplate) 300 is maintained at a desired temperature in all the positions of the sample carrier (e.g., the microplate carrier) 310 in the incubation chamber 320. The incubation chamber 320 can be constructed from material that well suited to maintain constant temperature, like continuous aluminum sheets, while still providing access to optical elements via small openings. The incubation chamber 320 is typically thermally insulated. The design of such chambers will be known to a person familiar with the art and from many multi-detection instruments. A common controlled temperature range may be from room temperature to the 65 C.

FIG. 23 is a diagram illustrating a multi-detection system according to an embodiment.

For live cells, the temperature is typically 37 C, but in addition control of gas around the sample is required. The control is accomplished by filling the complete housing 1910 of the instrument of FIG. 23 with appropriate gas mixture. The design avoids trying to contain the gas controlled environment to just measurement chamber or separation partitions. The aim of the design is to allow atmosphere within the housing 1910 to equalize. The design of the housing 1910 is thus made as gas tight as feasible by avoiding gaps in the housing and using soft gasketing material around user access doors.

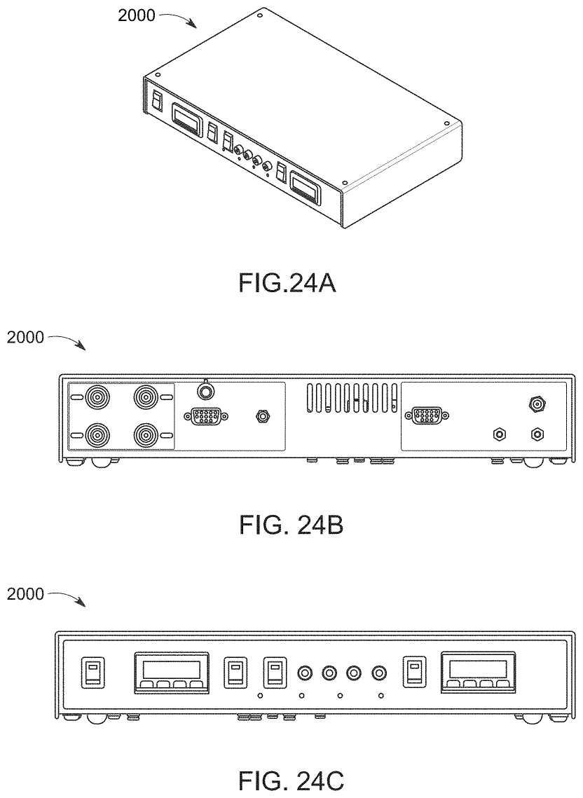

FIGS. 24A-24C is a diagram illustrating a gas control subsystem according to an embodiment.

Referring to FIGS. 24A-24C, an environmental control subsystem 2000 (e.g. a gas control subsystem) may be disposed external to the instrument. The environmental control subsystem 2000 allows a user to set CO2 and/or O2 concentration levels within the chamber to be different from a normal atmosphere: higher CO2 and lower O2. A gas sampling line connects the environmental control subsystem 2000 to the inside of the instrument housing. Based on composition of gas sampled or extracted from the instrument via the sampling line, the control systems may adjust flow of the CO2 or N2 gas being fed into the instrument, for example by the incoming gas being dispersed with small fan. This allows placement of all gas sensors and valves external to the main instrument and keeping complexity and reliability of gas control within external gas controller.

The combination of incubation chamber around the XY carrier travel zone and gas control of the atmosphere inside the housing, and thus around the sample carrier (e.g., the microplate), provides user with ability to run long term live cell experiments.

Referring to FIG. 23, an outside view of the overall instrument and elements subject to user interaction with the instrument as implemented in the example embodiment is shown. The carrier for the sample carrier (e.g., the microplate carrier) 310 presents itself to the user (shown at right) and a microplate 300 is placed onto the carrier for the sample carrier (e.g., the microplate carrier) 310, for example by a user or robotics arm, and is then positioned within the multi-detection system. The access to confocal cubes 1530, wide-field LED cubes 1201, and wide field filter cubes 1210, confocal disk modules and objectives 1230 is via the front of the instrument via door 1905. Thus, facilitating the user access to most user changeable elements at once.

According to certain embodiments, objectives (e.g. objective 1230 or objective 2210) of the present disclosure may be fluid immersion objectives.

A way to improve optical performance in microscopy is to use fluid immersion objectives. In light microscopy, a fluid immersion objective is a specially designed objective lens used to increase the resolution of the microscope. According to embodiments of the present disclosure, the optical system is an inverted microscope, meaning that the objective is located under the sample and views the sample from underneath. In inverted microscope arrangements of the present disclosure, when performing fluid immersion, a drop of fluid (e.g. water or other fluid) is put on the objective and is held in place by the surface tension of the fluid. The objective is then brought to the sample, where the droplet is sandwiched between the sample and the objective. In this way, the light passing to and from the sample to the objective does not go through air. The higher refractive index of the fluid over air results in increased numerical aperture. This increases resolution and increases the signal level. According to embodiments, the objective may be brought to the sample, and then the drop of fluid is put on the objective.

In addition to water immersion objectives, objectives of the present disclosure may be provided with other types of fluid for increasing numerical aperture. Some examples of the fluid include, for example, oil and glycerol. In embodiments of the present disclosure, the fluid may be water, oil, glycerol, or some other type of fluid that would increase the refractive index.

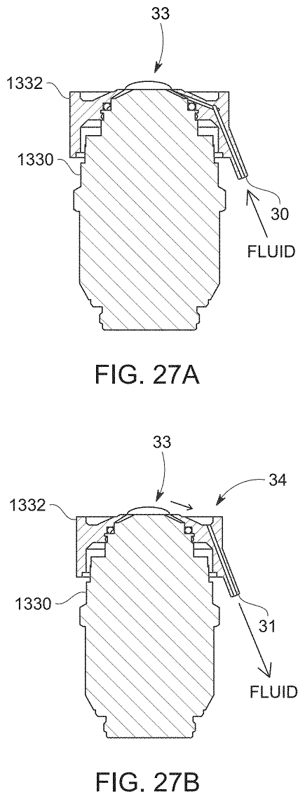

With reference to FIGS. 27A-27B, a liquid immersion objective according to embodiments of the present disclosure is described below. According to embodiments, an objective 1330 may be provided with a sleeve 1332 that fits over the objective 1330. The sleeve 1332 may be configured to provide a fluid path in and out of the sleeve 1332. In addition, the sleeve 1332 helps hold a fluid droplet 33 in place. According to embodiments, the sleeve 1332 has a port for pumping fluid in and a port for pumping the fluid out. According to embodiments, as shown in FIGS. 27A-27B, the inlet and outlet port may be a same port 31. With reference to FIG. 27B, liquid droplet excess 34 may exit the sleeve 1332 through the port 31. In an example embodiment, the sleeve 1332 may be formed of, for example, anodized aluminum, plastic, or other materials.

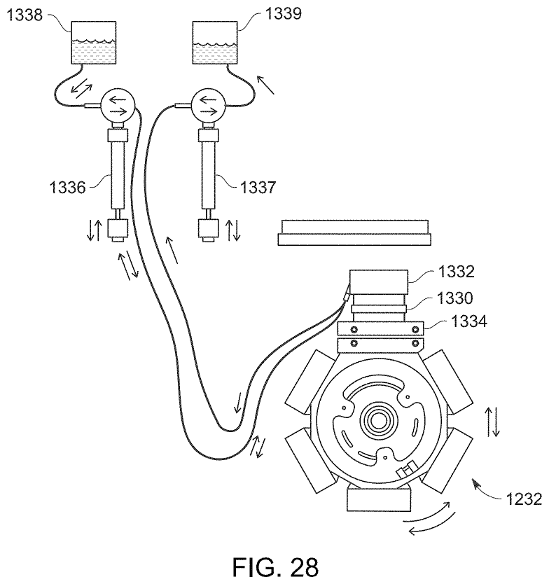

According to embodiments, with reference to FIG. 28, a fluid pump system may provided. The fluid pump system may include a first pump 1336, a second pump 1337, a first reservoir 1338 (a source reservoir), and a second reservoir 1339 (a waste reservoir), The fluid may be pumped by the first pump 1336 from the first reservoir 1338 to the head of the objective 1330. As shown in FIG. 28, the first pump 1336 may be a syringe pump. The fluid is then removed from the objective 1330 via the second pump 1337 pumping the fluid to the second reservoir 1339. The second pump 1337 may be referred to as a waste pump and may also be a syringe pump, as shown in FIG. 28. The first pump 1336 and the second pump 1337 may be other types of pumps that achieve the same or similar functionalities. The sleeve 1332 may be fit to the objective 1330, guide the fluid to the top of the objective 1330, and help to hold the fluid droplet in place. The sleeve 1332 may also have a waste port in which the fluid may be configured to be removed from the sleeve 1332. The objective 1330 may be a specially designed objective optimized for fluid (e.g. water) immersion application. In FIG. 28, the first reservoir 1338 and the second reservoir 1339 are shown as separate source and waste reservoirs, respectively. However, according to embodiments, a single reservoir may be provided, instead of the two separate reservoirs, in which the fluid could be reused. Additionally, the pumps may be multipurpose. For example, the BioTek C10 product has a fluidics dispense module that may be used to dispense reagents into the sample. This same dispense module could be configured to have additional purposes (including the purpose of the first pump 1336 and/or the second pump 1337) so as to reduce cost.

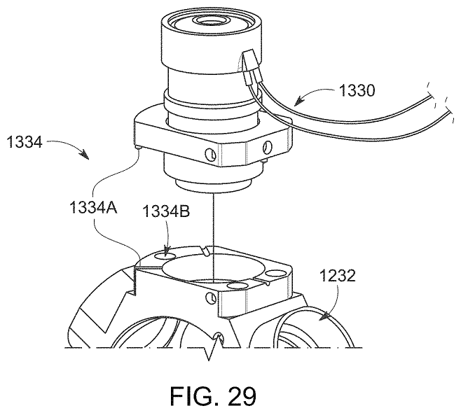

With further reference to FIG. 28, the objective 1330 may be attached to the objective turret 1232 by an objective coupling 1334. Description of the objective coupling 1334 is provided below with reference to FIG. 29.

As shown in FIG. 29, the objective coupling 1334 may include kinematic connections 1334A and magnets 1334B that are configured to couple together the objective 1330 and the objective turret 1232. For example, the objective 1330 may be provided with at least one from among a protrusion or recess as a first part of kinematic connections 1334A, and the objective turret 1232 may be include at least one of the other from among the protrusion or recess as a second part of the kinematic connections 1334A that corresponds to the first part. The magnets 1334B may be provided with one or more of the objective 1330 and the objective turret 1232. According to embodiments, both the objective 1330 and the objective turret 1232 may be provided with the magnets 1334B that correspond to each other and are configured to connect to each other via a magnetic force. In other embodiments, only one from among the objective 1330 and the objective turret 1232 may be provided with the magnets 1334B, which may be configured to connect to a magnetic material (e.g. a metal) provided with the other from among the objective 1330 and the objective turret 1232.

According to comparative embodiments, objectives may be screwed into an objective turret. However, the use of a sleeve and tubing with an objective may make screwing the objective into an objective turret difficult in at least some embodiments. The use of an objective coupling 1334 that includes kinematic connections 1334A and magnets 1334B, according to embodiments of the present disclosure, enables an objective with a sleeve and tubing to be easily installed.

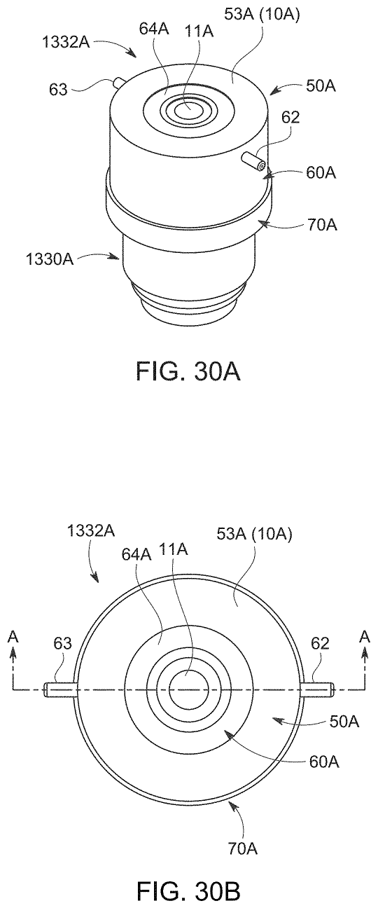

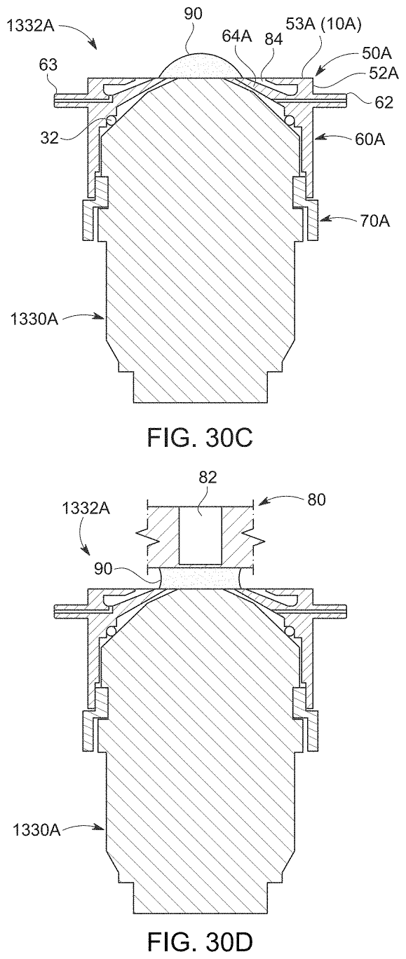

According to embodiments, with reference to FIGS. 30A-33C, the objective 1330 and sleeve 1332 may have various configurations. According to embodiments, the sleeve 1332 may also be referred to as a cap.

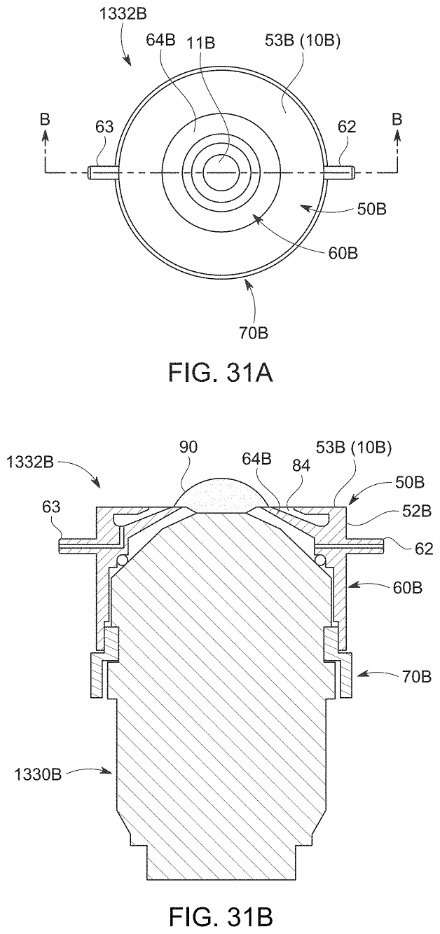

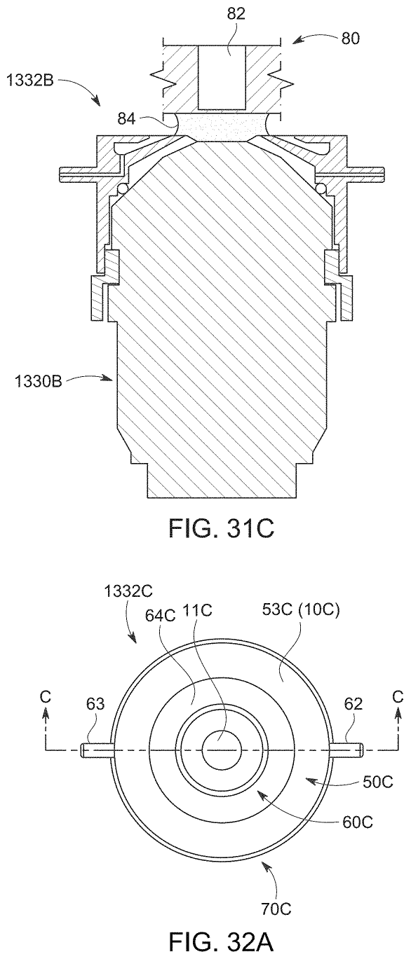

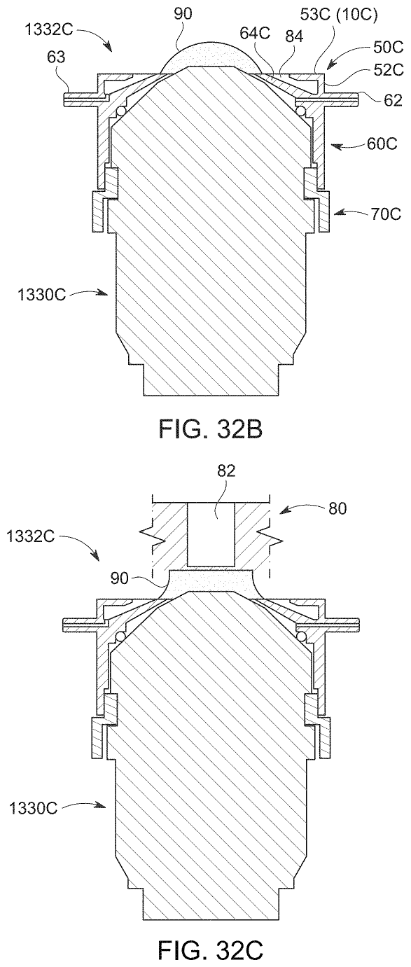

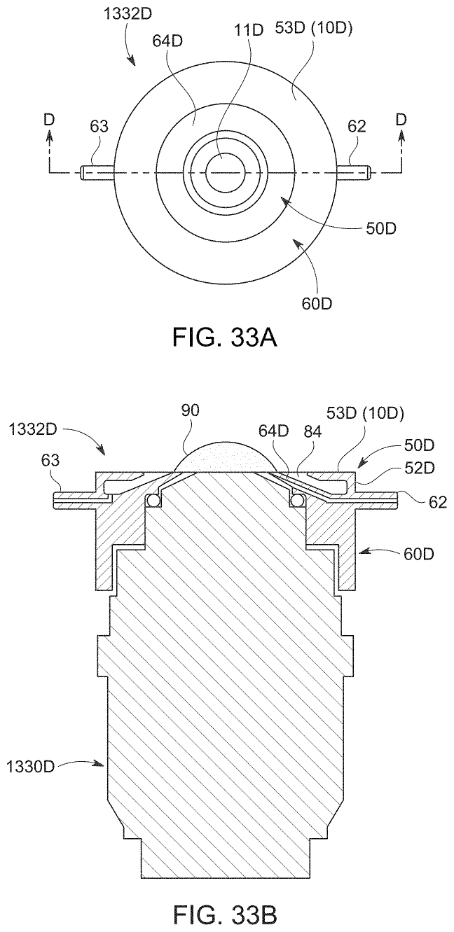

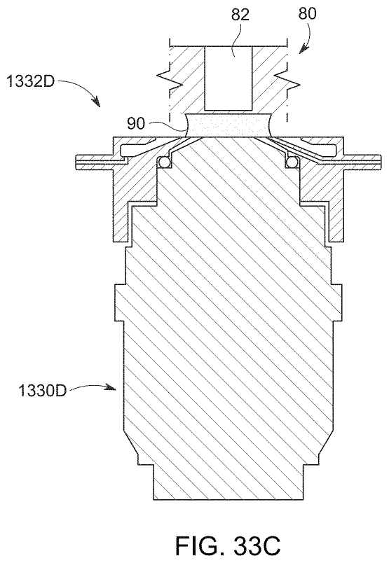

FIG. 29 is a diagram illustrating an objective coupling according to an embodiment; FIG. 30A is a perspective view illustrating a liquid immersion objective according to a first embodiment; FIG. 30B is a top view illustrating the liquid immersion objective according to the first embodiment; FIG. 30C is a first cross-sectional view, taken along line A-A in FIG. 30B, illustrating the liquid immersion objective according to the first embodiment in a state in which a liquid bulb is provided; FIG. 30D is a second cross-sectional view, taken along line A-A in FIG. 30B, illustrating the liquid immersion objective according to the first embodiment, over which a sample carrier (e.g., a microplate) is provided; FIG. 31A is a top view illustrating a liquid immersion objective according to a second embodiment; FIG. 31B is a first cross-sectional view, taken along line B-B in FIG. 31A, illustrating the liquid immersion objective according to the second embodiment, in a state in which a liquid bulb is provided; FIG. 31C is a second cross-sectional view, taken along line B-B in FIG. 31A, illustrating the liquid immersion objective according to the second embodiment, over which a sample carrier (e.g., microplate) is provided; FIG. 32A is a top view illustrating a liquid immersion objective according to a third embodiment; FIG. 32B is a first cross-sectional view, taken along line C-C in FIG. 32A, illustrating the liquid immersion objective according to the third embodiment, in a state in which a liquid bulb is provided; FIG. 32C is a second cross-sectional view, taken along line C-C in FIG. 32A, illustrating the liquid immersion objective according to the third embodiment, over which a sample carrier (e.g., a microplate) is provided; FIG. 33A is a top view illustrating a liquid immersion objective according to a fourth embodiment; FIG. 33B is a first cross-sectional view, taken along line D-D in FIG. 33A, illustrating the liquid immersion objective according to the fourth embodiment, in a state in which a liquid bulb is provided; and FIG. 33C is a second cross-sectional view, taken along line D-D in FIG. 33A, illustrating the liquid immersion objective according to the fourth embodiment, over which a sample carrier (e.g., a microplate) is provided.

In the below description of FIGS. 30A-33C, the same or similar features are given the same or similar reference characters. For purposes of clarity, redundant descriptions of same or similar features may be omitted.

With reference to FIGS. 30A-30D, a top surface 10A of a sleeve 1332A may be flush with a top surface 11A of a lens of an objective 1330A, and the sleeve 1332A may be configured to clamp to the objective 1330A.

The sleeve 1332A may include, for example, an upper portion 50A, a middle portion 60A, and a lower portion 70A. According to embodiments, the upper portion 50A, middle portion 60A, and the lower portion 70A may be separately or integrally provided with each other so as to constitute a single body or a plurality of bodies. According to embodiments, two from among the upper portion 50A, middle portion 60A, and the lower portion 70A may be integrally provided so as to constitute a single body, while the other from among the upper portion 50A, middle portion 60A, and the lower portion 70A may be separately provided as a separate body that is configured to attach to the other two. According to embodiments, the upper portion 50A, the middle portion 60A, and/or the lower portion 70A may be subdivided into separate bodies, and/or additional bodies may be provided. According to embodiments, any number of the upper portion 50A, the middle portion 60A, and the lower portion 70A may be formed of aluminum.

According to embodiments, any number of the upper portion 50A, the middle portion 60A, and the lower portion 70A may be formed to substantially exhibit rotational symmetry around a center axis of the objective 1330A. The center axis may be, for example, an optical axis of the objective 1330A.

The middle portion 60A may be provided above the lower portion 70A. The middle portion 60A may include an inlet port 62 and an outlet port 63. Fluid may be pumped into the sleeve 1332A via the inlet port 62, and pumped out of the sleeve 1332A via the outlet port 63, by a fluid pump system (e.g. refer to FIG. 28). The inlet port 62 and the outlet port 63 may be provided separately from each other, on opposite sides of the sleeve 1332A. However, the position of the inlet port 62 and the outlet port 63 is not limited to such configuration, and may be variously modified. According to embodiments, the inlet port 62 and the outlet port 63 may be constituted by a single port.

The middle portion 60A may further include a tapered portion 64A that follows a contour of the objective 1330A. For example, the tapered portion 64A may extend upwards and radially inward from an outer portion of the middle portion 60A. The tapered portion 64A may be formed to substantially exhibit rotational symmetry around the center axis of the objective 1330A. According to embodiments, the tapered portion 64A may have shapes other than a taper, so long as the shape follows a contour of the objective 1330A. The shape (e.g. inverted “V” shape that follows a contour of the objective 1330A) of the tapered portion 64A enables a liquid droplet 90 to have a desired shape on the objective 1330A for liquid immersion. According to embodiments, the tapered portion 64A may alternatively be referred to as a protruding portion.

According to embodiments, the inlet port 62 may include a passageway that extends through the tapered portion 64A, to an internal side of the tapered portion 64A, such as to be configured to supply the liquid for the liquid droplet 90 into a space between the objective 1330A and the tapered portion 64A.

The upper portion 50A may include a body. For example, the body may include a side wall 52A that extend upwards from the middle portion 60A, and a top wall 53A that extends radially inwards from the side wall 52A. The side wall 52A and the top wall 53A may substantially extend at 90 degrees from each other. However, an angle is not limited thereto, and may be variously modified according to embodiments. The body, including the side wall 52A and the top wall 53A, may be formed to substantially exhibit rotational symmetry around the center axis of the objective 1330A.

A groove 84 may be formed by and between the upper portion 50A and the middle portion 60A. For example, the groove 84 may be defined by an inner surface of the top wall 52, an inner surface of the side wall 53, and an outer surface of the tapered portion 64A. According to embodiments, the groove 84 may be formed to substantially exhibit rotational symmetry around the center axis of the objective 1330A. The groove 84 may be configured to receive and contain excess amounts of the liquid. According to embodiments, the groove 84 may communicate with the outlet port 63, such that excess amounts of the liquid in the groove 84 exit the sleeve 1332A via a passageway of the outlet port 63 that communicates with the groove 84.

With reference to FIGS. 30C-30D, at least an upper surface of the top wall 53A may constitute the top surface 10A of the sleeve 1332A that is flush with the top surface 11A of the lens of the objective 1330A. According to embodiments, a top surface of the tapered portion 64 may also be flush with the top surface 11A of the lens of the objective 1330A.

According to embodiments, one or more o-rings 32 may be provided between the sleeve 1332A and the objective 1330A. For example, an o-ring 32 may be provided between the middle portion 60A and the objective 1330A. The o-ring 32 may be configured to seal a bottom-side of the space in which liquid is received between the objective 1330A and the tapered portion 64A.

With reference to FIG. 30D, a sample carrier (e.g., a microplate) 80, that holds a sample in at least one well 82, may be provided directly above the sleeve 1332A and the objective 1330A. The liquid droplet 90 on the lens of the objective may come into contact with a bottom surface of the sample carrier (e.g., the microplate) 80, at a position directly below the well 82. The sample carrier (e.g., the microplate) 80 may correspond to, for example, sample carrier (e.g., microplate) 300 described in the present disclosure, or other sample carriers (e.g., microplates).

With reference to FIGS. 31A-31C, a top surface 10B of a sleeve 1332B may be above a top surface 11B of a lens of an objective 1330B, and the sleeve 1332B may be configured to clamp to the objective 1330B.

The sleeve 1332B may include, for example, an upper portion 50B, a middle portion 60B, and a lower portion 70B.

The middle portion 60B may include a tapered portion 64B, and the upper portion 50B may include a body that includes a side wall 52B and a top wall 53B. At least an upper surface of the top wall 53B may constitute the top surface 10B of the sleeve 1332B that is above the top surface 11B of the lens of the objective 1330B. According to embodiments, a top surface of the tapered portion 64B may also be above the top surface 11B of the lens of the objective 1330B, and flush with the top surface of the top wall 53B.

With reference to FIGS. 32A-32C, a top surface 10C of a sleeve 1332C may be below a top surface 11C of a lens of an objective 1330C, and the sleeve 1332C may be configured to clamp to the objective 1330C.

The sleeve 1332C may include, for example, an upper portion 50C, a middle portion 60C, and a lower portion 70C.

The middle portion 60C may include a tapered portion 64C, and the upper portion 50C may include a body that includes a side wall 52C and a top wall 53C. At least an upper surface of the top wall 53C may constitute the top surface 10C of the sleeve 1332C that is below the top surface 11C of the lens of the objective 1330C. According to embodiments, a top surface of the tapered portion 64C may also be below the top surface 11C of the lens of the objective 1330C, and flush with the top surface of the top wall 53C.

With reference to FIGS. 33A-33C, a top surface 10D of a sleeve 1332D may be flush with a top surface 11D of a lens of an objective 1330D, and the sleeve 1332D may be configured to screw onto the objective 1330D.

According to an embodiment, an internal surface of the sleeve 1332D and an external surface of the objective 1330D may include screw threads that correspond and engage with each other such that the sleeve 1332D and the objective 1330D can be attached to and detached from each by a rotating motion of at least one of the sleeve 1332D and the objective 1330D.

The sleeve 1332D may include, for example, a first portion 60D and a second portion 50D.

The first portion 60D may include a tapered portion 64D, and the second portion 50D may include a body that includes a side wall 52C and a top wall 53C. At least an upper surface of the top wall 53D may constitute the top surface 10D of the sleeve 1332D that is flush with the top surface 11D of the lens of the objective 1330D. According to embodiments, a top surface of the tapered portion 64D may also be flush with the top surface 11D of the lens of the objective 1330D.

According to embodiments, an internal surface of the first portion 60D and may include the screw threads.

According to embodiments, the top surface 10D of the sleeve 1332D may be above or below the top surface 11D of the lens of the objective 1330D. For example, the top surface of the top wall 53D may be above or below the top surface 11D of the lens of the objective 1330D, and the top surface of the tapered portion 64D may be flush with the top surface of the top wall 53D.

According to embodiments of the present disclosure, various embodiments of confocal microscopy may be alternatively or additionally provided. For example, a laser point scanning confocal system may be provided. Laser point scanning confocal microscopy may include focusing a single point of laser light through a small aperture (pinhole) and scanning sequentially across the sample point by point in a zig-zag pattern. The sample fluoresces, and the light is sent back through the optical system. The light then may be read point by point by a detector, which may be a Photo Multiplier Tube (PMT) but could also be detected using other light measurement sensors. The signal from the sensor may be recorded point by point, and each point may constitute a single pixel in an image. There are advantages and disadvantages to a laser point scanning system over a spinning disk confocal. Laser point scanning systems have typically been slower than spinning disk confocals and thus, in many cases, were not appropriate for high throughput applications or live cell images. On the other hand, laser point scanning confocal systems penetrate deeper in the sample and provide better axial and lateral resolution. Recently, there have been improvements made to laser point scanning systems to increase speed and thus are starting to rival spinning disk speeds while still providing increased depth penetrations. The speed of the laser point scanning confocal system is limited by the scanning speed of the motors that drive a scanning mirror of the system.

According to embodiments, confocal subsystems of the present disclosure may comprise both a laser point scanning confocal and a spinning disk confocal. The spinning disk confocal system may be used for live sample imaging and high throughput application, while the laser point scanning confocal system may be used to penetrate deeper into a sample with increased resolution. Like how one could use wide field imaging or other measurement modalities to provide a “hit”, embodiments of the present disclosure may implement spinning disk confocal to quickly scan through a 3D sample and locate some point of interest. The laser point scanning system may then be used to take a more detailed image of the area of interest. Both laser point scanning confocal systems and spinning disk systems are available on the market as two separate instruments. However, there are several problems with using two separate instruments in such a manner. For one, the cost of both spinning disk and laser confocal microscopes would make putting a workflow as described above impractical. Additionally, there is the technical problem of relocating to a region of interest on an alternate microscope. With both a laser point scanning confocal system and a spinning disk system implemented in a same instrument, a “hit” could be found, and then the optical system could switch and scan the region of interest without moving the stage. Finally, there is also an issue of studying live cells, whereby the sample changes over time. Moving a sample to a different instrument takes too long relative to the speed of the changing biology. When moving the sample to another instrument, the “hit” region of interest may have changed and may no longer be relevant.

Another advantage to having both a laser point scanning confocal and a spinning disk confocal in a same instrument is that one can leverage the laser point scanning confocal system, not for imaging, but for targeting a specific area of the sample to photobleach it. The laser point scanning confocal system and specific control over an X-Y scanning mirror, provided therein, allows for targeting of a very small and specific area of the sample with the laser. This may be one spot or a block defined in a zig-zag scanning. Then, once the photobleaching has occurred, the instrument may be quickly switched to the spinning disk confocal to monitor the Fluorescence Recovery after PhotoBleaching (FRAP). Some specific applications include: (a) analysis of molecule diffusion within the cell (e.g. studying F-Actin diffusion in primary dendritic cells after a region of interest has been photobleached); (b) quantifying fluidity of bio membranes (e.g. membrane fluidity in C. elegans); and (c) analysis of protein binding (e.g. monitoring dynamic binding of chromatin proteins in vivo).

The pinpoint accuracy of laser point scanning confocal systems combined with the speed of imaging of a spinning disk system, according to embodiments of the present disclosure, solves an unmet market need in FRAP assays.

With reference to FIGS. 30A-30B, a configuration according to embodiments of the present disclosure that includes a laser point scanning confocal system, a spinning disk confocal system, and wide field functionality in a single instrument is described below. However, embodiments of the present disclosure may include any combinations of the above systems and functions.

FIG. 30A illustrates a case where the instrument is set to the laser point scanning confocal (LSC) modality. FIG. 30B illustrates a case where the instruction is set to the wide field or spinning disk confocal modality. According to embodiments, a mechanism may be provided to switch between the LSC system and the wide field or spinning disk confocal system. As shown in FIGS. 30A-30B, elements in block 2220 are movable and enable the switch between laser point scanning optics and spinning disk/confocal. For example, block 2220 may be a plurality of disk modules which may be moved for selection between disks (and therefore modalities) as described in the present disclosure.

With reference to FIG. 30A, embodiments of the present disclosure may include a laser point scanning confocal system. Light, typically from a laser source, enters such system at a light input device 2201. The light input device 2201 may be, for example, a fiber-coupled input or a directly coupled laser without a fiber. The light is then collimated when passed through a lens 2202. The light then hits the long pass dichroic 2203. The long pass dichroic 2203 is designed to reflect the input light and allow for the passing of the emission light at a high wavelength. It is typical that the light source would have multiple input wavelengths.

Embodiments of the present disclosure may support an automated means of switching the long pass dichroic 2203 to accommodate the input wavelength. The light is then reflected off the scan mirror 2204. The scan mirror 2204 may be controlled with two-axis motors 2205 and 2206. In some embodiments, the motors are both Galvo type motors and, in other embodiments, one motor is driven by Galvo, and the other motor is a resonant scanner. The resonant scanner is much faster than the Galvo motor but allows for less control over the positioning. Both types of motors are known to those skilled in the art. According to embodiments, the scan mirror 2204 may configured as a plurality (e.g. two) of separate scan mirrors. For example, the plurality of separate scan mirrors may include a first mirror configured for x-scanning and a second mirror configured for y-scanning, wherein positioning of each of the separate scan mirrors may be, for example, controlled by a respective motor.

After the light is reflected off the scan mirror 2204, the light then goes through a focusing lens 2207, and then a tube lens 2208. The light then travels to a reflecting mirror 2209, objective 2210, and finally to sample 2211, wherein a spot illuminated on the sample may be tiny. Then, assuming that the sample is fluorescent, the light travels backward through the laser point scanning system, and goes to the long pass dichroic 2203. Provided that the emission light is in the passband of the long pass dichroic 2203, it will pass through to the focusing lens 2213 and then through a pinhole 2214. The pinhole 2214 may be a single-size pinhole, or it may be variable in size. Variation in size may be achieved by having multiple pinholes on a selector wheel or a variable iris. The light then goes through lens 2215 and then to dichroic 2216.

The arrangement shown in FIG. 30A includes a dual PMT 2218 setup that would enable the measurement of multiple emission wavelengths simultaneously. The arrangement could be extended out to be an additional number of PMTs 2218. It could also be a single PMT 2218 arrangement where the emission wavelength is selected via a version of the dichroic 2216 and EM filter 2219 that includes a switching mechanism. The switching mechanism could be a cube and a slider or multiple wheels, both of which would be understood by those skilled in the art.

In laser point scanning systems, the light input device 2201 location may need precise alignment with the pinhole 2214. This makes implementation, installation, and maintenance of a laser point scanning system challenging. It is typical that, after shipment or maintenance, adjustment may need to be made to realign the pinhole 2214 to fiber location. A solution to this problem is that both the light input device 2201 (e.g. fiber optic input) and pinhole 2214 are on a motorized axis, and the instrument (e.g. controller thereof) can automatically align the light input device 2201 and the pinhole 2214 by controlling corresponding motors. Such aspect may provide benefits for after shipment, maintenance, or even with thermal changes in the instrument. In addition, the fiber input location may be smaller than the pinhole size so that there is some margin in the design. With automated alignment, the pinhole size could be reduced and thus increase confocality of the system, thereby increasing resolution and sample penetration.

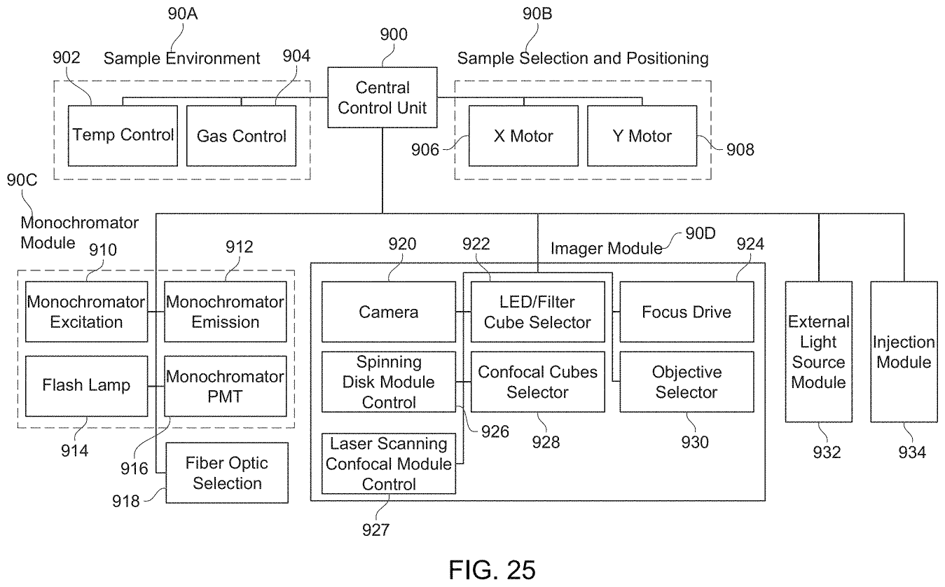

FIG. 25 is a functional block diagram that illustrates the control of modalities of instruments according to embodiments.

The operation of modalities may be controlled by a central control unit (e.g., processor, CPU, microprocessor, etc.). According to embodiments, the central control unit may also be referred to as a controller (e.g. controller 1000).

The central control unit 900 may be connected to communicate with and control elements of embodiments of the present disclosure. For example, the central control unit 900 may be connected to communicate with and control elements of the sample environment 90A, elements of sample selection and positioning 90B, elements of the monochromator module 90C, elements of the imager module 90D, an external light source module 932, and an injection module 934.

Elements of sample environment 90A under control may provide temperature control (902) and gas control (904) as described above.

Sample selection and positioning 90B may be controlled through the use of motors for positioning samples in any X and Y directions (906 and 908).

Elements of the monochromator module 90C under control may include monochromator excitation (910), monochromator emission (912), monochromator PMT (916), fiber optics selection (918), and light sources such as a flash lamp 914.

Elements of the imager module 90D under control may include an objective selector 930, an image capturing device such as camera 920, a focus drive 924 for objectives, LED and filter cube selector 922 for wide field imaging, confocal cubes selector 928, and spinning disk module and control (926) (e.g. selection and focusing), and laser scanning confocal module control (927).

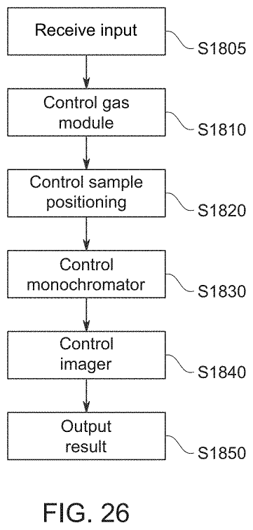

FIG. 26 is a flowchart of control method of a multi-detection system according to an example embodiment.

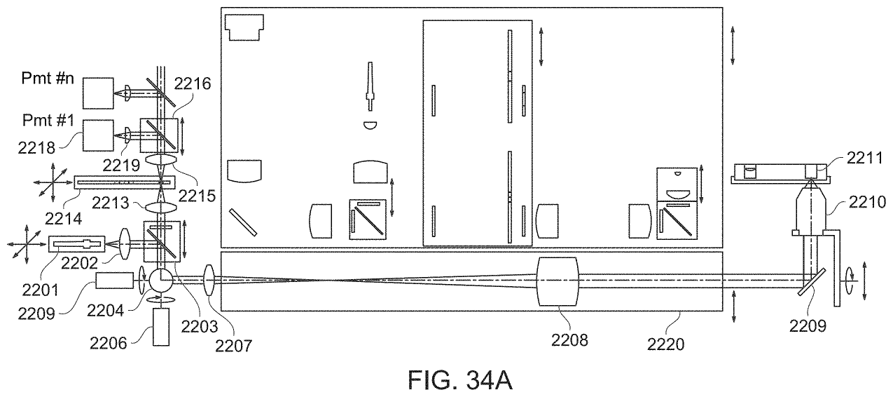

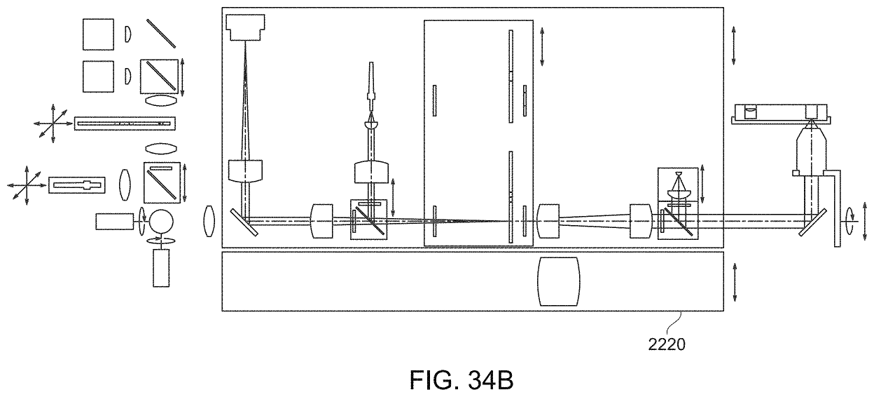

Control of the instrument may be coordinated through use of the controller, as discussed above with respect to, for example, FIG. 25 and/or FIGS. 34A-34B. Input to the instrument (step S1805) may be accomplished through a local user interface of the instrument, such as a touch pad or graphical display, or through communication with the instrument over a wired or wireless connection, such as over a network.

In the case of input to the instrument, input may be performed through the use of a user interface or graphical user interface displayed on a computer or other terminal that executes a control application.

The input may be user input, such as setting and parameters for executing control of the instrument.

In response to receiving input, control of the instrument may be effectuated through the various elements of the instrument as, for example, discussed above regarding FIG. 25 and/or FIGS. 34A-34B. For example, in response to receiving user input, the instrument may be controlled to execute a gas control procedure of the gas module (step S1810), a sample positioning control procedure to control positioning of samples (step S1820), a monochromator control procedure to control operations of the monochromator (step S1830), an imager control procedure to control the imager (step S1840), and to output a result of the controlling of the elements of the instrument (step S1850).

Although control is presented as illustrated in FIG. 26, elements may be individually controlled in any sequence, and control of all elements is not required. Accordingly, the multiple modalities of the instrument may be controlled in a single assay.

The control method illustrated in FIG. 26, and other functions described herein that may be performed by a controller, may be implemented through execution of a processing unit (e.g., CPU) controlling elements of the instrument by executing one or more control programs. The programs may be stored in a memory (i.e., RAM, ROM, flash, etc.), or other computer-readable medium (i.e., CD-ROM, disk, etc.). The program may be executed locally by the instrument, or by a control apparatus, such as a computer that transmits commands to be executed by the instrument.

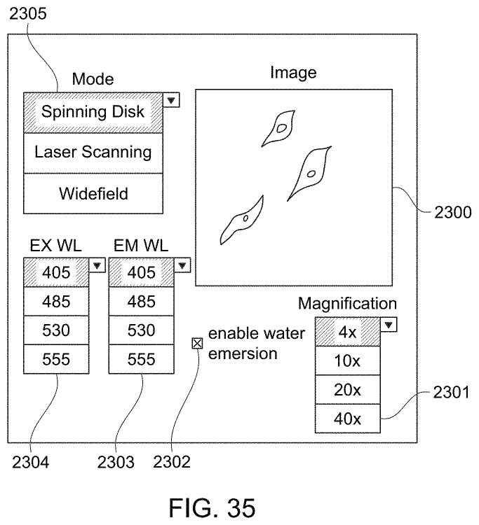

With reference to FIG. 35, embodiments of the present disclosure may include a display, and the controller may be further configured to cause the display to display a user interface. FIG. 35 illustrates an example of the user interface in a case where the instrument has a combination of various optical modes. Element 2300 is an image of the sample. Element 2301 is a drop-down menu for selecting a magnification. Element 2302 is a selection box to enable/disable water immersion. If selected, and the objective is configured for water immersion, the controller may cause water to be automatically pumped to the objective and may automatically remove water when imaging is completed or the check box of element 2302 is deselected. Element 2303 is a drop-down list for the EM wavelength selection. FIG. 35 illustrates that a selection between 4 different EM wavelengths may be provided, but any number of EM wavelength selections may be provided. Element 2304 is a drop-down list for the EX wavelength selection. FIG. 35 illustrates that a selection between 4 different EX wavelengths may be provided, but any number of EX wavelength selections may be provided. Element 2305 is a drop-down menu allowing one to select between the various modes of the instruction. FIG. 35 illustrates selection between modalities, where the system include spinning disk, laser scanning, and wide field modalities. According to embodiments, the modalities listed in element 2305 may depend on the modalities present in the system. The system may, for example, have any combination of the above-mentioned modalities (and/or additional modalities), or only a single modality. In a case where only a single modality is provided, element 2305 may not be provided. According to embodiments, elements 2301, 2302, 2303, 2304, and 2305 are not limited to being drop-down menus and selection boxes, and may indicate options for selection in any manner known to a person of ordinary skill in the art.

According to embodiments, the interface may include display elements that enable a user to select a plurality of modalities to automatically be performed in a sequence. For example, based on one or more inputs from a user with respect to the interface, the controller may be configured to control the sequence to automatically be performed. The sequence may include any order of modality operations, including the orders of modality operations described in the present disclosure. For example, an operation using the spinning disk or wide field imaging system and then an operation using the laser point scanning confocal system may be performed.

Components and features of the optical module are further described in U.S. Pat. No. 7,782,454 titled “Universal multidetection system for microplates,” incorporated herein by reference in its entirety for all purposes.

For instance, according to one aspect, there is provided an optical module which includes a first optical device that transmits a narrow waveband of light and includes a first filter and a first monochromator that provide different paths for the narrow waveband of the light. The optical module may also include a light source that generates the light as broadband excitation light, wherein the first optical device transmits a narrow waveband of the broadband excitation light and blocks other wavebands of the broadband excitation light through the first filter or the first monochromator; a second optical device that directs the narrow waveband of the broadband excitation light onto the sample and receives emission light from the sample; a third optical device that transmits a narrow waveband of the emission light; and a detector that converts the narrow waveband of the emission light into an electrical signal; wherein the third optical device includes a second filter and a second monochromator that provide alternative paths for the narrow waveband of the emission light.

Transfer Module

In accordance with certain embodiments, the analytical instrument further comprises a transfer module configured to transfer optical signals from the array of sensor units to the signal processing module. For example, the transfer module may transfer one or more of excitation, reference, and emission optical signals.

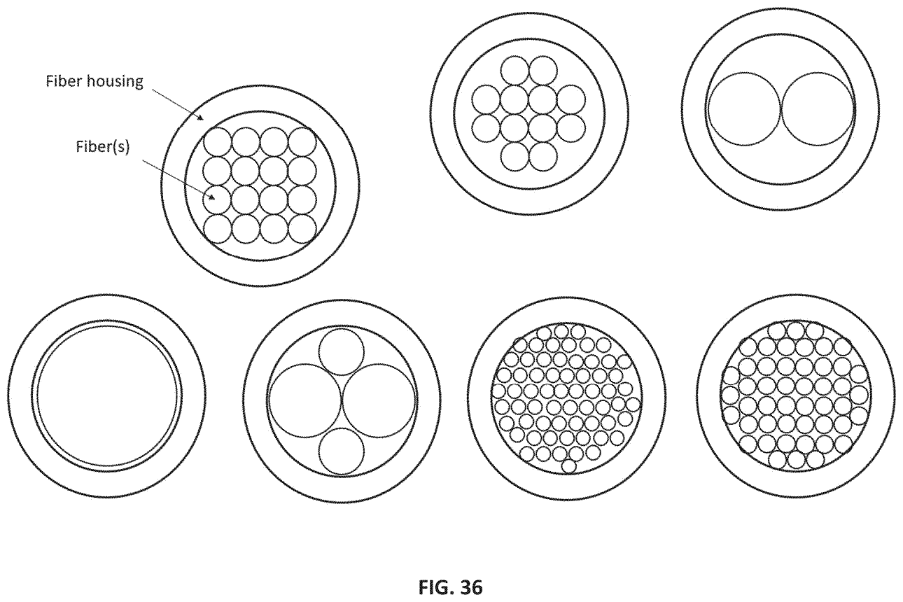

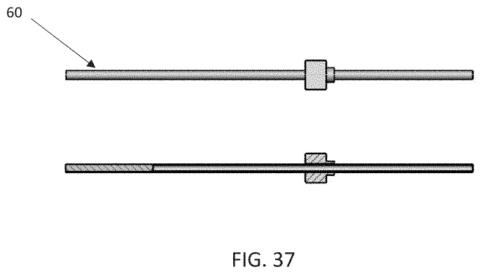

The transfer module may be formed of a multiplexed fiber optic material. FIGS. 36-37 are diagrams showing several views of an exemplary transfer module 60, including a side view (FIG. 37) and a cross-sectional view (FIG. 36) of transfer module 60. In embodiments, the transfer module 60 may comprise an array of fiber optic bundles, each fiber optic bundle in communication with a corresponding sensor unit of the array of sensor units. The fiber optic bundles may be positioned and arranged to directly interface with one or more sensor units. Each fiber optic bundle may be formed of an array of fiber optic cables contained within a fiber and/or plastic probe housing, e.g., a metal fiber probe housing or a plastic housing, as shown in the cross-sectional view of FIG. 36.

In certain embodiments, the transfer module may be in the form of a homogenized fiber optic wave guide optically connecting the sensor units to the transfer module, e.g., each sensor unit to a corresponding fiber optic bundle of the transfer module. The homogenized fiber optic wave guide may be configured to uniformly distribute light onto one or more sensor units. The homogenizer may improve mechanical and optical shuffling.

### EXAMPLES

The embodiments may be further understood with reference to the following examples. The examples are intended to serve as an illustration and are not limiting.

### Example 1: Exemplary Protocol

Cells are seeded in the assay wells of a multi-well sample carrier (e.g., microplate) at a confluency of 50-90%. Suspension cells are attached to the well bottom to maximize sensitivity. A 96-well plate constructed and arranged to mate with the apparatus is used for this exemplary protocol. However, the sample carrier (e.g., the multi-well plate) may have any number of wells corresponding with the apparatus, e.g., 1, 6, 8, 12, 24, 36, 48, 64, 72, 96, 192, 384 or others. Temperature of the cell suspensions is controlled.

The apparatus lowers the sensor probes into the assay wells. The sensors are positioned 200 microns above the well bottoms, forming transient microchambers, also referred to as “measurement chambers” herein, of approximately 2 microliters. As the oxygen and pH levels change, the changes are measured by the sensors. Measurements are typically made for a predetermined amount of time between 1 minute and 5 minutes, for example, 3 minutes. Rate changes are calculated automatically by a computing device. Upon completion of this measurement period the sensor probes are raised, allowing the extracellular medium to come back to baseline conditions.

The sensor cartridge also contains ports (4 per well) to enable injection of modulators (target analytes) into the cell wells during the assay. When specified by the instrument protocol, for example, as provided by via graphical user interface, the controller instructs the dispensing system to inject a test compound into the assay wells and perform a gentle mixing step to ensure distribution of the compound throughout the assay medium. All wells are processed in this manner simultaneously. Subsequent measurement cycles, any additional injections specified by the protocol, and rate calculations are performed automatically.

The exemplary protocol was executed with THP-1 cells (human monocytes derived from a patient with acute monocytic leukemia) for test purposes. OCR and ECAR data were measured and reported using an analytical instrument as described herein. The test was also executed in a comparative analytical instrument having conventional temperature control, signal processing, and motion actuator motor components. The results are presented in the graphs of FIGS. 10A-10D.

The graph of FIG. 10A shows OCR measurement values over assay time as measured with an analytical instrument as disclosed herein. The graph of FIG. 10B shows OCR measurement values over assay time as measured with the comparative analytical instrument. The graph of FIG. 10C shows ECAR measurement values over assay time as measured with an analytical instrument as disclosed herein. The graph of FIG. 10D shows ECAR measurement values over assay time as measured with the comparative analytical instrument.

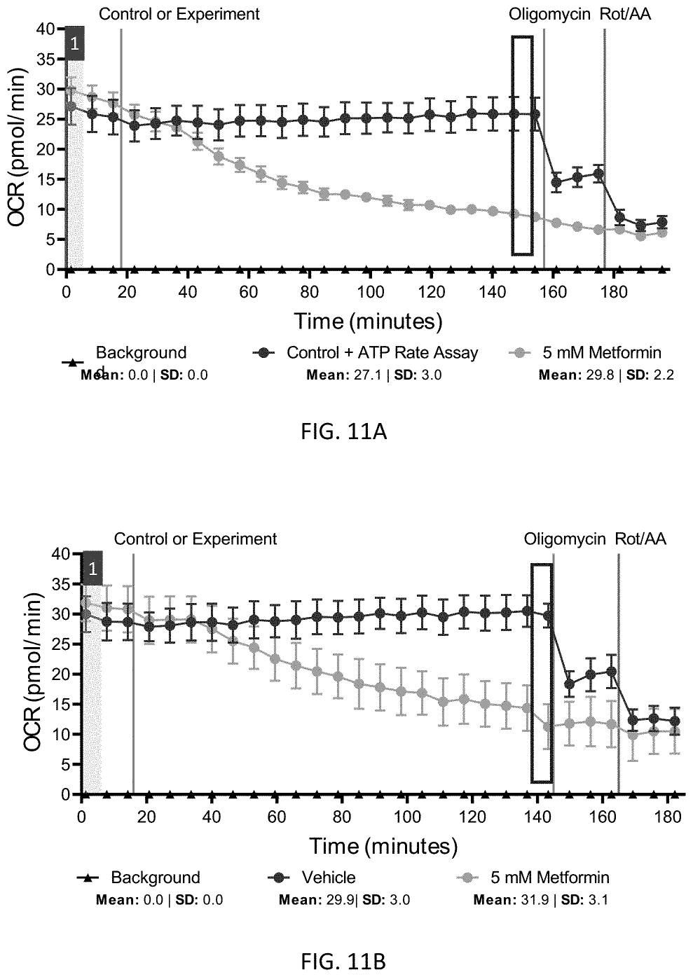

The exemplary protocol was also executed with A549 cells (human lung cancer cells) and administration of 5 mM Metformin as a target agent. OCR data was measured using an analytical instrument as described herein and the comparative analytical instrument. The results are presented in the graphs of FIGS. 11A-11B.

The graph of FIG. 11A shows OCR measurement values over assay time as measured with an analytical instrument as disclosed herein. The graph of FIG. 11B shows OCR measurement values over assay time as measured with the comparative analytical instrument.

Accordingly, the analytical instrument having temperature control elements, a signal processing module, and a motion actuator assembly motor as described herein showed a significant improvement in lower limit OCR detection precision and readability over the comparative analytical instrument while simultaneously detecting ECAR. While not wishing to be bound by theory, it is believed that the improvement in uniformity of temperature across samples within a controlled temperature zone may improve both the performance of the analytical instrument in sensing target analytes as well as the performance of the biology of the cells.

### Example 2: Evaporation Test Protocol with Water Samples

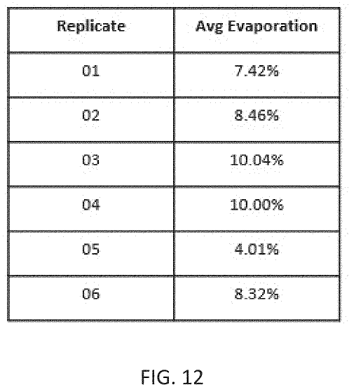

Six assays were run in the analytical instrument disclosed herein using known volumes of water in the sample carrier (e.g., the multi-well plate) for 6 hours with a modified protocol configured to take 4 measurements per hour. Evaporation from the water samples was measured using a plate reader. A standard curve was created by measuring the absorbance of known volumes of water. Immediately following each assay, absorbance measurements of the test plates were collected. The standard curve was used to calculate the volume of water in each well of the test plates to evaluate the amount of water volume lost to evaporation during the 6-hour assay. The results were calculated as a percentage of total volume lost. Average evaporation for each assay is presented in the table of FIG. 12.

As shown in the table of FIG. 12, the greatest average percentage of water volume lost to evaporation during a 6-hour assay was 10.04%. Accordingly, the volume of sample fluid lost to evaporation was low.

While not wishing to be bound by theory, it is believed that the improvement in uniformity of temperature across samples within a controlled temperature zone may reduce evaporation of sample fluid, improving both the performance of the analytical instrument in sensing target analytes as well as the performance of the biology of the cells.

### Example 3: Direct Identification of Mitochondrial Toxicity Using a Novel Index Derived from Mitochondrial Oxygen Consumption Rates

Mitochondrial toxicity (MitoTox) is a common issue with therapeutic development, contributing to drug/compound/substance candidate attrition and post-market drug/compound/substance withdrawals (Wallace, K. B., 2008. Mitochondrial off targets of drug therapy. Trends Pharmacol. Sci. 29, 361-366). Among the methods used to assess drug/compound/substance-induced mitochondrial toxicity in drug/compound/substance discovery and pre-clinical safety, direct measurement of mitochondrial oxygen consumption using the Agilent Seahorse XF technology has been well documented as a specific and sensitive marker/indicator (Yvonne Will & James Dykens (2014) Mitochondrial toxicity assessment in industry—a decade of technology development and insight, Expert Opinion on Drug Metabolism & Toxicology, 10:8, 1061-1067, DOI: 10.1517/17425255.2014.939628) (Tilmant K.a,*, Gerets H.a, De Ron P.a, Hanon E.a, Bento-Pereira C.a,b,l, Atienzar F. A In vitro screening of cell bioenergetics to assess mitochondrial dysfunction in drug development. Toxicology in Vitro 52 (2018) 374-383).

Thus, disclosed herein is a standardized XF solution that allows for assessment of compounds exhibiting mitochondrial toxicity. As described herein, the XF Pro analyzer has several novel design features that provide enhanced sensitivity, precision, and consistency. Here we exploit these improvements to detect drug/compound/substance-induced mitochondrial dysfunction using for OCR measurements.

The Agilent Seahorse XF Mito Tox Assay workflow includes sequential injections of oligomycin and FCCP, but includes a separate control group that is provided rotenone/antimycin A prior to the assay. Compounds to be assessed for mitochondrial toxicity are provided to the cells at a designated time prior to the assay.

Based on responses in either basal, oligomycin and/or FCCP OCRs of the test compounds compared to appropriate controls, the XF Mito Tox Assay can identify 3 distinct types of mitochondrial toxicity: direct/indirect inhibition of the ETC or other mitochondrial processes, uncoupling of the ETC from OxPhos, and (potential) specific inhibition of the OxPhos machinery (CV, ANT, PiT).

Significant improvements are provided through the extraction of a novel parameter, a Mito Tox Index (MTI), derived from oxygen consumption rates (OCRs) measured by an analytical instrument as disclosed herein (the Agilent Seahorse XF Analyzer).

This approach of deriving an easily interpretable mitochondrial toxicity metric is surprisingly enabled by the improvements in sensing precision achieved through implementation of the instrument and methods disclosed herein, providing an easy and robust way to screen and validate toxicity in vitro. The workflow enables the reduction of complex respirometric responses to a Mitochondrial Toxicity Index (MTI) metric, providing two types of MTI scoring the inhibitor effect on the electron transport chain (ETC) and the uncoupler effect on negative and positive scales respectively. The inhibitor MTI is designed to calculate the relative inhibitory effect on the maximal OCR to the effect of the ETC inhibitor control, rotenone/antimycin A mix. In contrast, the uncoupler MTI is to calculate the relative elevation in minimal OCR measured after the oligomycin injection to the FCCP effect in the vehicle control group. Among the compounds showing no significant score in either MTIs, potential ATP synthase inhibitors can be identified by monitoring the basal OCR-specific suppression since ATP synthase inhibitors do not affect the maximal OCR. The capacity to derive a defined metric enables additional functionality such as the convenient generation of dose response relationships or convenient threshold setting for ‘hit’ identification.

Defining the Mitochondrial Toxicity Index (MTI)

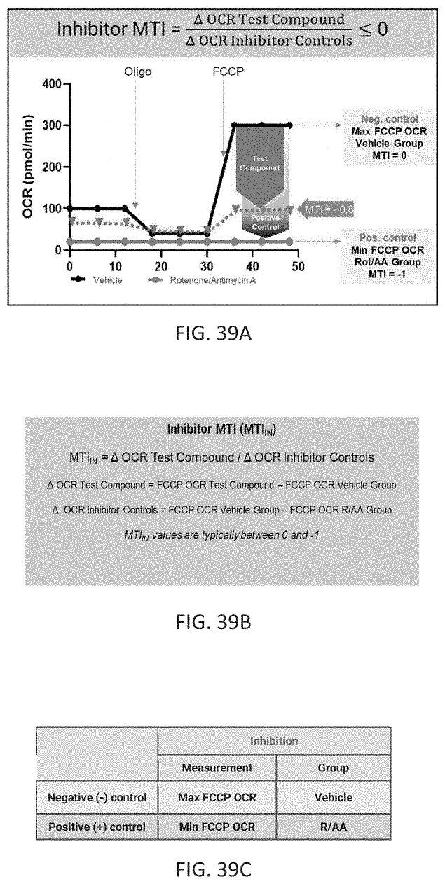

In order to discriminate among the three modes of mitochondrial toxicity described above, as well as to quantitate the magnitude of toxicity, the Mito Tox Index (MTI) value was derived leveraging the increased measurement precision enabled by the instrument disclosed herein. Mito toxicity due to inhibition, where inhibition is defined and detected as a decrease in FCCP OCR of the test compound compared to maximal FCCP OCR of the vehicle group, results in a negative MTI value (typically between 0 and −1) and is illustrated and described in FIGS. 39A-39C.

FIG. 39A refers to a scenario where a test compound results in a decrease in FCCP induced OCR, compared to Vehicle (neg) control (MTI=0), then the compound is categorized as an Inhibitor, with a negative MTI value (e.g. MTI=−0.8). Note that Rot/AA OCR serves as a positive (+) control for inhibition (MTI=−1). FIGS. 39B-39C provide a summary of measurements and groups used for inhibition controls.

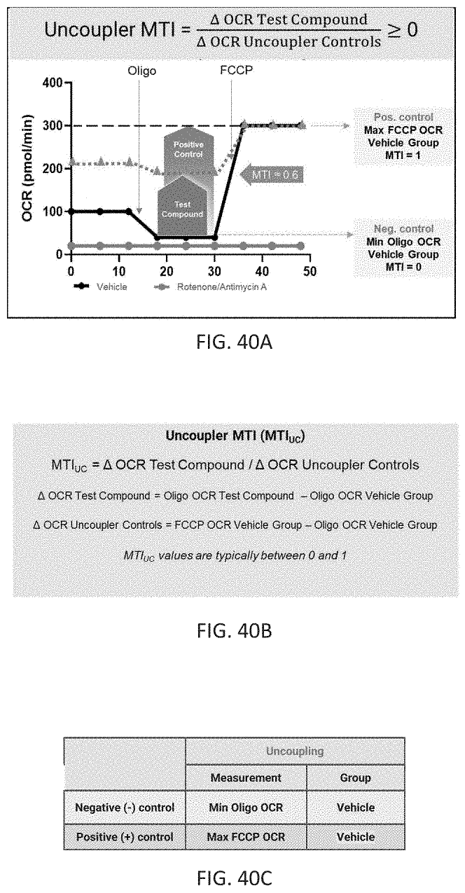

Mito toxicity due to uncoupling, where uncoupling is defined and detected as an increase in Oligo OCR of the test compound compared to minimal Oligo OCR of the vehicle group, results in a positive MTI value (typically between 0 and 1) and is illustrated and described in FIGS. 40A-40C.

FIG. 40A refers to a scenario where a test compound results in an increase in Oligo induced OCR, compared to Vehicle (neg) control (MTI=0), then the compound is categorized as an Uncoupler, with a positive MTI value (e.g. MTI=0.6). Note that Vehicle FCCP OCR serves as a positive (+) control for uncoupling (MTI=1). FIGS. 40B-40C provide a summary of measurements and groups used for uncoupling controls.

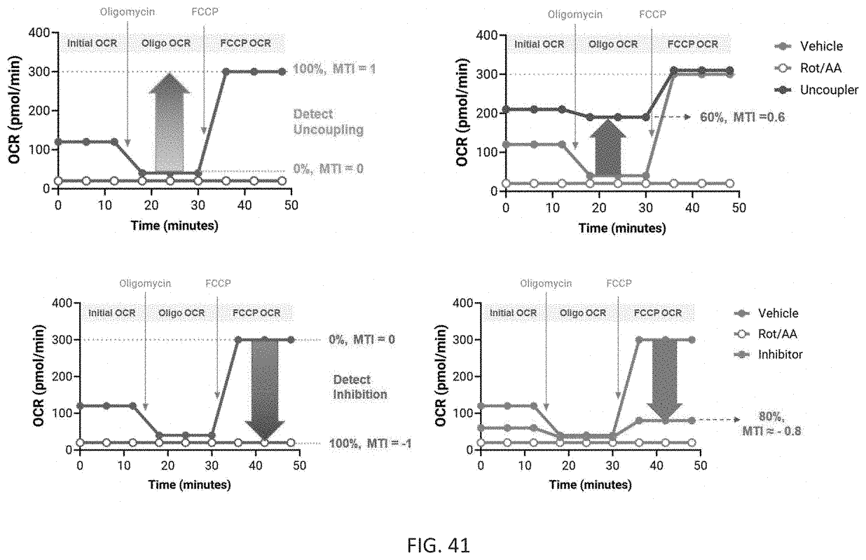

In summary, the MTI is the fraction value of test compound effect compared to respective controls for either uncoupling and/or inhibition. The Uncoupler MTI is calculated as positive index number and is defined as the fraction of uncoupling elicited by a test compound compared to maximal uncoupling (FCCP OCR of the vehicle group, positive control). Note that Oligo OCR of the Vehicle group serves as the negative control for uncoupling. Conversely, the Inhibitor MTI is calculated as negative index number and is defined as the fraction of inhibition elicited by a test compound compared to maximal inhibition. (FCCP OCR of the Rot/AA group, positive control). Note that FCCP OCR of the Vehicle group serves as the negative control in this case. Upon transformation, both uncoupler and inhibitor MTIs can be generated for each well. Exemplary MTI detection graphs are shown in FIG. 41.

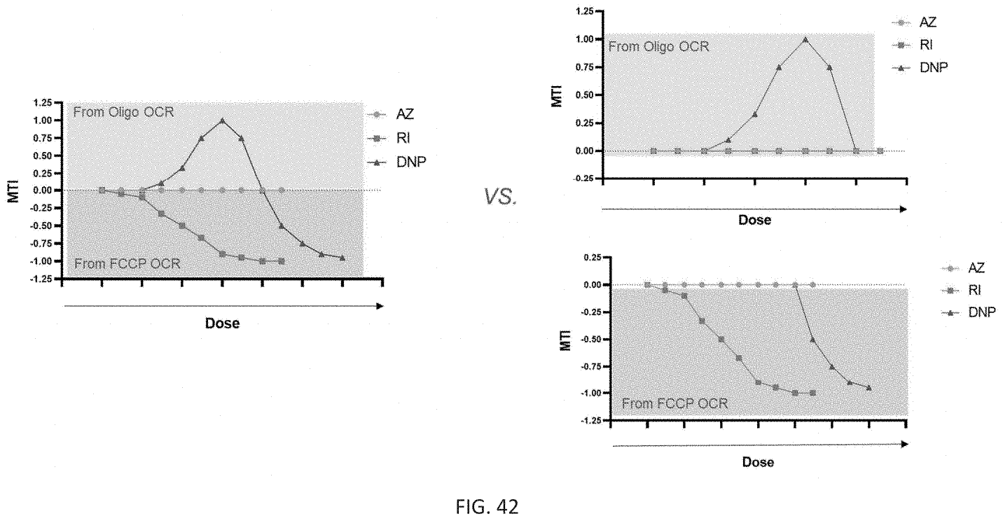

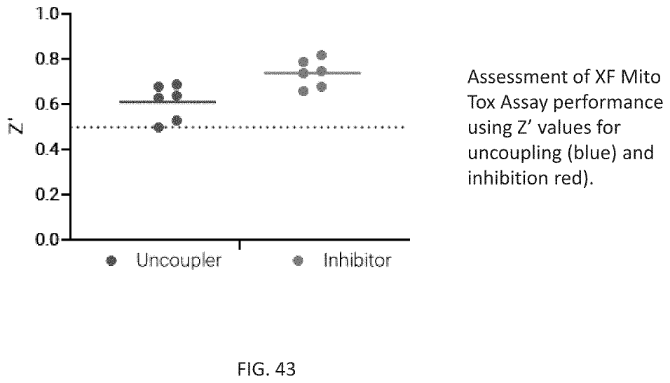

A specific case of mito tox due to decreased of mito function is the direct inhibition of the ATP synthase (CV), or other components of the OxPhos machinery (e.g. ANT, Pi transporter). This type of inhibition often shows a decrease in basal OCR, while oligo and FCCP OCRs are significantly less affected (FIGS. 42A-42D). If a test compound treatment results in a decrease in Basal OCR, compared to Vehicle (neg) control (MTI=0), BUT does not result in significant decrease in maximal/FCCP induced OCR then the compound is categorized as an OPI.

XF Mito Tox Assay Performance Metrics

The Z-factor is used as a measure of assay quality or assay performance (Zhang). Z′ factors are typically between 0 and 1.0 and may be interpreted as follows: Z′=1.0 is considered ideal assay performance. If 0.5<Z′<1.0, this is considered an excellent assay, meaning greatly decreased chances of reporting false positive or false negative results. If 0.0<Z′<0.5, this is considered to be marginal assay performance, with increased chances of reporting false positive and negative results. If the Z′ is less than 0, there is too much overlap between the positive and negative controls for the assay to be useful.

Z-factor can therefore be used as a measure of the quality or power of a screening assay. (Note Z′ is not the same as the z-score). In a screening campaign, there is typically a comparison of large numbers of single measurements of unknown samples to well-established positive and negative control samples. The purpose of the assay is to determine which, if any, of the single measurements are significantly different from the controls. To this end, the distribution of measurements from the positive control, negative control, and the other single measurements must be considered in order to determine the probability that each measurement may have occurred by chance. Further, these distributions cannot be determined a priori, the performance must be assessed post assay to show/predict that the assay would be useful in a screening (or user defined) setting. The greater the Z′ value, the less chance that the assay is reporting false positives and/or false negatives.

In the XF Mito Tox Assay, corresponding Z′ Factors are provided for both Uncoupling and Inhibition to allow assessment of assay performance, as each has respective positive and negative control. The Z′ Factor is calculated as follows:

Z′=1−[3(mean of pos control+mean of neg control)/(Std Dev of pos control−Std Dev of neg control)]

Surprisingly, the precision improvements of the instrument described here-in enable a simplifying Mito Tox metric (MTI) that is capable of achieving excellent Z′ values (>0.5) even in the absence of cell normalization (where data are corrected to accounted for variation in cell growth across a sample carrier (e.g., a microplate)) (FIG. 43).

### Examples of Use

This performance means that the XF Mito Tox Assay may be performed as a compound screen (e.g., up to 80 individual compounds at a single dose per plate) or used to perform dose response assays (e.g. 8 compounds, 10 concentrations/compound per plate). When used together with respective software tools, resulting kinetic OCR data is automatically transformed into MTI values for each test compound.

Modes of Mito Tox detected and measured using the XF Mito Tox Assay were calculated. Drugs/compounds/substances that exert effects on transport, TCA, FAO, ETC (drugs/compounds/substances that result in decreases FCCP induced) OCR are categorized as Inhibitors. Drugs/compounds/substances that act as protonophores which uncouple the ETC from the OxPhos that result in increases in Oligo OCR are categorized as Uncouplers. Drugs/compounds/substances that cause inhibition of the OxPhos machinery (ATP synthase, ANT, Pi transporter) and result in decreases in Basal OCR only are categorized as “OPIs”.

Dependent on the context/goal of the mito tox investigation, test compounds may be further subject to dose response assays including dose response curves and IC50/EC50 values. FIG. 42 shows kinetic dose response OCR data for 3 compounds, which were then transformed to MTI values for each dose and plotted vs. compound concentration (FIG. 42). IC50 (or EC50) values were calculated for each sample.

### Experimental Methods

All cell lines were maintained according to manufacturer recommendations. HepG2 cells were seeded in XF Pro Moat cell culture sample carrier (e.g., microplates) at a density of 2.0×10⁴ cells per well and cultured in DMEM low glucose (Gibco 11885) supplemented with 2 mM Glutamax and 10% serum. All cells were incubated overnight at 37° C., 5% CO₂. The following day, cells were washed twice with Mito Tox Assay Media (XF DMEM pH 7.4 plus 10 mM XF Glucose, 1 mM XF Pyruvate, and 2 mM XF Glutamine) and incubated at 37° C., no CO₂, for 60 minutes. Pretreatment solutions were added at the time of cell washing. Cell plates were then transferred to XF Pro analyzers for assay performance, using sequential injection of oligomycin (1.5 μM), FCCP (1.5 μM). rotenone/antimycin A (0.5 μM each)(final concentrations). Where assessed, cells were then counted using a Cytation 5 instrument.

All XF assays were performed as described in the XF Mito Tox Kit User Guide, including compound dilutions and sensor cartridge preparation. Agilent Seahorse Analytics is a web-based software platform that provides a simple, streamlined data analysis workflow for the XF Mito Tox assay. Seahorse Analytics was used to calculate key parameters of the XF Mito Tox assay—Mito Tox Index (MTI value) and/or IC50/EC50 values. Instructions to perform data analysis using Seahorse Analytics User Guide.

### Example 4: The Presently Disclosed Analyzer Exhibits Improved Measurement Precision, Relative to a Comparative Analytical Instrument

THP-1 cells were cultured in RPMI cell culture medium (supplemented with 10% FBS, 10 mM glucose, 2 mM glutamine, and 1 mM pyruvate) at 37 C in 5% CO₂. Cell density was maintained below 10⁶ cells/mL and culture medium was refreshed every 48-72 hours.

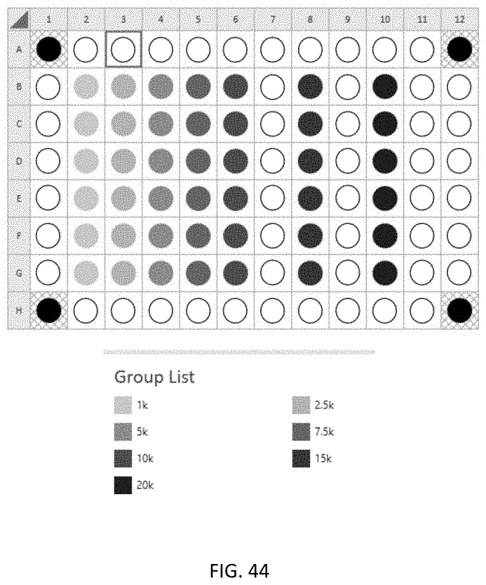

The cell suspension was transferred to a centrifuge tube, and the cells were centrifuged at 1000×g for 10 minutes. Cells were resuspended in an assay medium consisting of RPMI supplemented with 1 mM HEPES buffer (wherein sodium bicarbonate was replaced with an osmotic equivalent concentration of NaCl) pH 7.4, 10 mM Glucose, 2 mM glutamine, and 1 mM pyruvate. The resuspended cells were diluted in separate tubes to concentrations of 2×10⁴, 5×10⁴, 1×10⁵, 1.5×10⁵, 2×10⁵, 3×10⁵, and 4×10⁵ cells/well. Six replicate wells were seeded for each concentration on each of two 96-well plates that were pre-coated with poly-D-lysine and pre-warmed at 37° C. overnight. 50 μL of resuspended cells were added to each well such that the final concentrations in each well were 1×103, 2.5×103, 5×103, 7.5×103, 1×10⁴, 1.5×10⁴, or 2×10⁴ cells/well as shown in FIG. 44. The 96-well plates were centrifuged at 200×g for 1 minute and assay medium was added such that each well had a final volume of 180 μL. The 96-well plates were incubated at 37 C in a non-CO₂ incubator for 30 min.

One of the 96-well plates was placed in an analytical instrument as described herein. The other plate was placed in a comparative analytical instrument having conventional temperature control, signal processing, and motion actuator motor components. Each instrument was programmed with command instructions. In this case, the instrument was programmed to take three measurements, inject each well with 20 μL of the solution from port A from a cartridge disposed above the cell sample in a well, conduct three measurements, inject each well with 22 μL of the solution from port B from a cartridge disposed above the cell sample in a well, and take a final three measurements. The instrument was programmed to take a measurement every six minutes, with each six minute interval comprising a three minute mixing step and a three minute measuring step.

An oligomycin solution was prepared to 15 μM in assay media. A mix solution of 5 μM rotenone and 5 μM antimycin A was prepared in assay media. Ports of a pre-hydrated cartridge for each well were loaded with the 15 μM oligomycin solution (Port A) and the 5 μM Rotenone+5 μM antimycin A solution (Port B). The hydrated assay cartridge containing the indicated reagents was loaded into the instrument and the experiment was performed according to the instrument protocol. The instrument measured OCR as described in the exemplary protocol of Example 1. Basal OCR was calculated as the average of the six replicate wells in each plate of the third measurement.

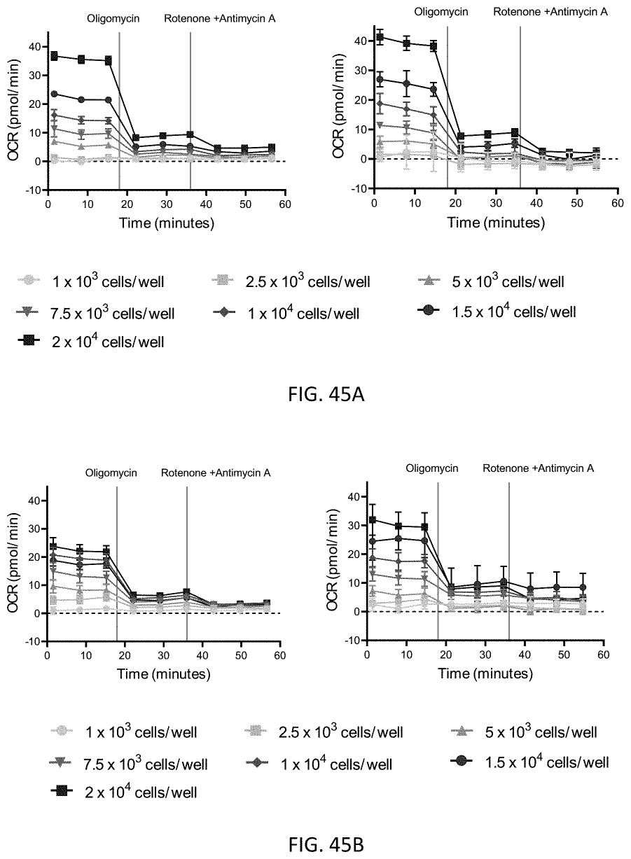

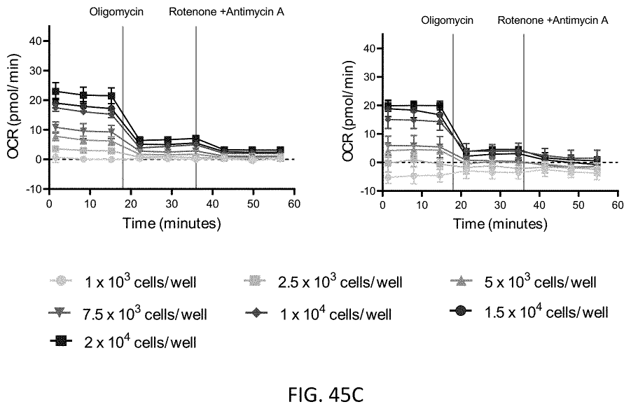

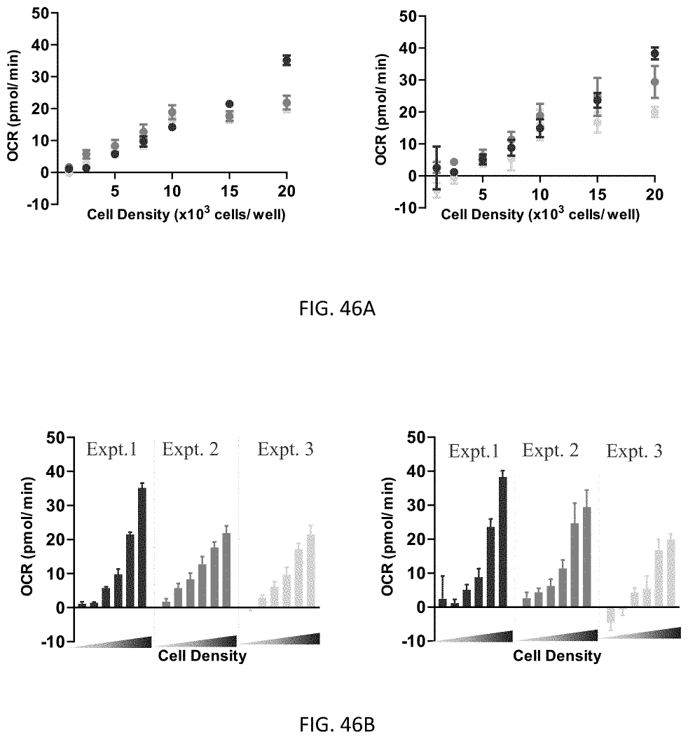

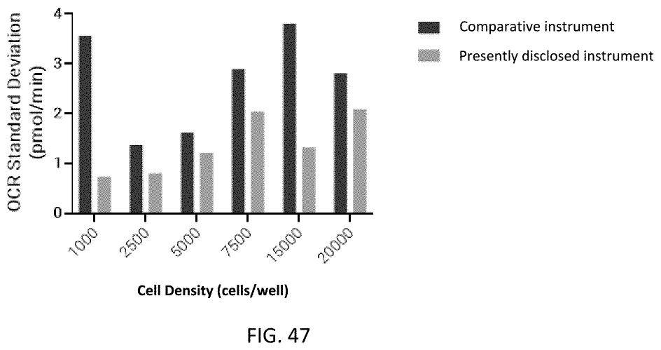

This experiment was performed three times and the results from each trial are shown individually in FIGS. 45A-45C. The basal OCRs from all the trials are summarized in FIGS. 46A-46B and the standard deviations across the trials are shown in FIG. 47. Collectively these data showed improved measurement performance of the instrument described herein, relative to the comparative analytical instrument at low oxygen consumption rates (OCR). Specifically, the data collected on the instrument described herein resulted in reduced occurrence of negative rates at low densities or after rotenone+antimycin A injections, lower standard deviations, reduced inter- and intra-plate variability, and more consistent measurements at low OCR. This was demonstrated using a combination of titrated seeding densities and with injections of mitochondria-inhibiting compounds. These improvements enabled more confident cell data interpretation due to repeatability and better resolution between assay groups.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. An analytical instrument comprising:
a sensing system comprising an array of sensor units configured to generate a first signal in response to a first analyte, e.g., at least one analyte proportional to O₂ content, and a second signal in response to a second analyte, e.g., at least one analyte proportional to pH value, each sensor unit of the array of sensor units positioned to correspond with a corresponding well on a sample carrier comprising an array of wells;
a stage configured to receive the sample carrier;
a motion actuator assembly configured to position the stage and/or the sensing system relative to one another on one or more of an x-axis, a z-axis, and a y-axis;
a sample temperature control element, e.g., a heating element, configured to control temperature of samples within at least one well (e.g., each well) of the sample carrier to be within a predetermined amount of a sample within another well of the sample carrier; and
a signal processing module operatively connected to the sensing system, e.g., configured to receive and/or amplify the first signal and the second signal.

2. The instrument of claim 1, wherein:
(i) the sensing system, the stage, the motion actuator assembly, the sample control element, and the signal processing module are contained within a housing, optionally wherein the housing comprises an opening on a side wall dimensioned to allow passage of the stage and the sample carrier;
(ii) the instrument further comprises the sample carrier, optionally wherein the sample carrier is disposed on the stage;
(iii) the motion actuator assembly comprises at least one axis actuator assembly, e.g., at least one x-axis actuator assembly, optionally wherein:
(a) the at least one axis actuator assembly, e.g., x-axis actuator assembly, is configured to position the stage relative to the sensing system on at least one axis, e.g., the x-axis, e.g., configured to align at least one well (e.g., each well) of the sample carrier disposed on the stage with a corresponding sensor unit on the x-axis, and/or
(b) the at least one axis actuator assembly, e.g., x-axis actuator assembly, is configured to position the stage relative to the housing on at least one axis, e.g., the x-axis, e.g., within the housing or exterior to the housing through the opening;
(iv) the motion actuator assembly comprises at least one y-axis actuator assembly, optionally wherein the at least one axis actuator assembly, e.g., y-axis actuator assembly, is configured to position the stage relative to the sensing system on at least one axis, e.g., the y-axis, e.g., configured to align at least one well (e.g., each well) of the sample carrier disposed on the stage with a corresponding sensor unit on the y-axis;
(v) the motion actuator assembly comprises at least one z-axis actuator assembly, optionally wherein the at least one axis actuator assembly, e.g., z-axis actuator assembly, is configured to position the sensing system relative to the stage on at least one axis, the z-axis, e.g., configured to position each sensor unit in fluid communication with the corresponding well; and/or
(vi) the sensing system is incorporated in or on a cartridge.

3-13. (canceled)

14. The instrument of claim 1, wherein:
(i) the instrument further comprises a dispensing system comprising at least one injector configured to dispense at least one target agent into one or more wells of the sample carrier optionally an injector motion actuator assembly positioned to drive the at least one injector to dispense the at least one target agent across a plurality of wells of the sample carrier;
(ii) the dispensing system comprises an array of injectors configured to dispense at least one target agent, each injector positioned to correspond with a corresponding well on the sample carrier, optionally wherein the array of injectors comprises more than one injector positioned to correspond with at least two wells (e.g., each well) on the sample carrier, e.g., at least 2, 3, 4, 5, 6, 7, 8, 9, or 10 injectors positioned to correspond with at least two wells (e.g., each well) on the sample carrier;
(iii) the instrument further comprises a manifold temperature control element, e.g., a heating element, configured to control temperature of the dispensing system, e.g., target agent, sensing system, and/or cartridge, optionally wherein:
(a) the manifold temperature control element and the sample temperature control element are configured to control temperature independently, and/or
(b) the manifold temperature control element is configured to control temperature of the target agent and/or the sensing system and/or the cartridge and the sample temperature control element is configured to control temperature of the samples within the array of wells of the sample carrier to be within 3° C., e.g., 2° C., 1° C., 0.6° C., 0.5° C., 0.4° C., 0.3° C., 0.2° C., or 0.1° C., of the corresponding target agent and/or the corresponding sensor unit;
(iv) the sample temperature control element and/or the manifold temperature control element is configured to control evaporation of the samples within the array of wells to be less than 25%, e.g., less than 20%, 15%, 12%, 10%, 9%, 8%, 7%, 6%, 5%, 4%, 3%, 2%, or 1%;
(v) the at least one axis actuator assembly, e.g., at least one z-axis actuator assembly, is configured to position the dispensing system relative to the stage on at least one axis, e.g., the z-axis, e.g., configured to position each injector in communication, e.g., fluid communication, with the corresponding well, allowing delivery of the target agent to the sample;
(vi) the at least one injector or each injector of the array of injectors is configured to dispense the same target agent;
(vii) each injector is configured to independently dispense a selected target agent, e.g., a first injector is configured to dispense a first target agent and a second injector is configured to dispense a second target agent, optionally one or more of a third injector is configured to dispense a third target agent, a fourth injector is configured to dispense a fourth target agent, and a nth injector is configured to dispense a nth target agent, optionally wherein a plurality of injectors or the array of injectors are configured to independently dispense more than one target agent into at least one well of the sample carrier;
(viii) the sensing system and the dispensing system are incorporated in or on the cartridge;
(ix) the instrument further comprises at least one target agent loaded in the dispensing system;
(x) the sample temperature control element and/or the manifold temperature control element is formed of a temperature conductive material, optionally wherein:
(a) the sample temperature control element is fixed to the stage,
(b) the sample temperature control element is configured to be in close proximity or direct contact with the sample carrier, and/or
(c) the manifold temperature control element is configured to be in close proximity or direct contact with the dispensing system;
(xi) the sample temperature control element is configured to control the temperature of samples within at least one well (e.g., each well) of the sample carrier to be 0° C.-70° C. above ambient temperature, e.g., 8° C.-20° C. above ambient temperature, e.g., 0, 1, 2, 3, 4, 5, 6, 7, 8, 9, 10, 11, 12, 13, 14, 15, 16, 17, 18, 19, 20, 30, 40, 50, 60, or 70° C. above ambient temperature;
(xii) the sample temperature control element is configured to maintain the temperature of samples within at least one well (e.g., each well) of the sample carrier to be within a predetermined range;
(xiii) the sample temperature control element is configured to control the temperature of samples within at least one well (e.g., each well) of the sample carrier such that a sensor signal in response to a level, production, or consumption of a target analyte, e.g., the first analyte or the second analyte, does not differ more than a predetermined amount between two identical or substantially identical samples, e.g., does not differ substantially between two identical or substantially identical samples, optionally wherein:
(a) the target analyte is O₂ and the sample temperature control element is configured to control the temperature of samples within at least one well (e.g., each well) such that the sensor signal in response to level, production, or consumption of the target analyte does not differ more than 10%, e.g., does not differ more than 5%, 3%, 1% or 0.1% between two identical or substantially identical samples, and/or
(b) the target analyte is the analyte proportional to pH value and the sample temperature control element is configured to control the temperature of samples within at least one well (e.g., each well) such that the sensor signal in response to level, production, or consumption of the target analyte does not differ more than 10%, e.g., does not differ more than 5%, 3%, 1%, or 0.1% between two identical or substantially identical samples;
(xiv) the sample temperature control element is configured to control the temperature of samples within at least one well (e.g., each well) of the sample carrier to be within 3° C., e.g., within 2° C., 1° C., 0.6° C., 0.5° C., 0.4° C., 0.3° C., 0.2° C., or 0.1° C. of the sample within another well of the sample carrier;
(xv) the manifold temperature control element is configured to control the temperature of sensors to be within 3° C., e.g., within 2° C., 1° C., 0.6° C., 0.5° C., 0.4° C., 0.3° C., 0.2° C., or 0.1° C. of another sensor; and/or
(xvi) the sample temperature control element and the manifold temperature control element are configured to control the temperature of samples within at least one well (e.g., each well) of the sample carrier to be within 3° C., e.g., within 2° C., 1° C., 0.6° C., 0.5° C., 0.4° C., 0.3° C., 0.2° C., or 0.1° C. of a corresponding sensor.

15-39. (canceled)

40. The instrument of claim 1, wherein:
(i) the sample temperature control element is configured to control the temperature of a sample within a first well to be within the predetermined amount of a sample within a second well, wherein the first well is a border well and the second well is an internal well of the sample carrier, wherein the border well is a well which has no other well disposed between the border well and an edge or border of the sample carrier;
(ii) the manifold temperature control element is configured to control the temperature of a sensor corresponding to a first well to be within the predetermined amount of a sensor corresponding to a second well, wherein the first well is a border well and the second well is an internal well of the sample carrier, wherein the border well is a well which has no other well disposed between the border well and an edge or border of the sample carrier;
optionally wherein:
(a) wherein the sample temperature control element and/or the manifold temperature control element is configured to maintain the temperature of samples within the first well and the second well and/or sensors corresponding with the first well and the second well to be within a predetermined range, and/or
(b) the sample temperature control element and/or manifold temperature control element is configured to control the temperature of samples (e.g., two identical or substantially identical samples) within the first well and the second well and/or sensors corresponding to the first well and the second well such that a sensor signal in response to level, production, or consumption of a target analyte, e.g., the first analyte or the second analyte, does not differ more than a predetermined amount between each sample, e.g., does not differ substantially between each sample, e.g., when the samples are analyzed under the same or substantially same conditions, further optionally wherein the target analyte is O₂ and/or the analyte proportional to pH value, and the sample temperature control element and/or manifold temperature control element is configured to control the temperature of samples (e.g., two identical or substantially identical samples) within the first well and the second well and/or sensors corresponding to the first well and the second well such that the sensor signal in response to level, production, or consumption of the target analyte does not differ more than 10%, e.g., does not differ more than 5%, 3%, 1%, or 0.1% between each sample, e.g., when the samples are analyzed under the same or substantially same conditions; and/or
(iii) the sample temperature control element is configured to control the temperature of samples within the first well to be within 3° C., e.g., within 2° C., 1° C., 0.6° C., 0.5° C., 0.4° C., 0.3° C., 0.2° C., or 0.1° C. of the sample within the second well and/or the manifold temperature control element is configured to control the temperature of a sensor correspond with the first well to be within 3° C., e.g., within 2° C., 1° C., 0.6° C., 0.5° C., 0.4° C., 0.3° C., 0.2° C., or 0.1° C. of a sensor corresponding with the second well;
optionally wherein the first well is a border well and the second well is an internal well of a sample carrier having 1 or more wells, e.g., 6, 8, 12, 24, 36, 48, 64, 72, 96, 192, 384 or more wells.

41-46. (canceled)

47. The instrument of claim 1, wherein:
(i) the sample temperature control element comprises a heating element;
(ii) the sample temperature control element forms a controlled temperature zone which comprises the array of wells of the sample carrier, optionally wherein:
(a) the controlled temperature zone does not comprise a headspace of the housing,
(b) a volume of the controlled temperature zone does not exceed a volume of the sample carrier by more than 1, 2, 3, 4, 5, 6, 7, 8, 9, or 10-fold,
(c) a volume of the controlled temperature zone does not exceed 10%, e.g., 9%, 8%, 7%, 6%, 5%, 4%, 3%, 2%, or 1%, of a volume of the housing,
(d) a temperature of components outside the controlled temperature zone is not substantially altered, e.g., increased or decreased, by activation of the sample temperature control element;
(iii) the sample temperature control element is configured to bring the temperature of samples within at least one well (e.g., each well) of the sample carrier to be within a predetermined range of a target temperature within about 5 hours, 3 hours, 1 hour, 45 minutes, 30 minutes, 15 minutes, 10 minutes, 5 minutes, 3 minutes, or 1 minute of activation of the sample temperature control element and/or introduction of the sample carrier into the controlled temperature zone;
(iv) the sample temperature control element and/or the manifold temperature control element is configured to control temperature to control, e.g., reduce, limit, or inhibit, diffusion of gases in the controlled temperature zone, optionally wherein the sample temperature control element and/or the manifold temperature control element is configured to control temperature to reduce, limit, or inhibit, the diffusion of gases in the controlled temperature zone such that a composition of gases in the controlled temperature zone does not vary significantly e.g., does not vary more than 1%, 2%, 4%, 6%, 8%, 10%, 12%, 14%, 16%, 18%, or 20%;
(v) the signal processing module is capable of operating at increased relative humidity (RH), e.g., at least 75% RH, 85% RH, 95% RH, or 99% RH;
(vi) the signal processing module is configured to receive and amplify the first signal and the second signal simultaneously;
(vii) the signal processing module is configured to receive and amplify the first signal and the second signal individually, e.g., sequentially;
(viii) the signal processing module is configured to detect one or more of the first signal and the second signal using time-based detection, e.g., rate of decay, phase shift, or anisotropy detection;
(ix) the signal processing module is configured to detect one or more of the first signal and the second signal using intensity-based detection, optionally including a ratiometric measurement;
(x) the signal processing module comprises a printed circuit assembly formed of an insulating material having a high dielectric constant;
(xi) the signal processing module comprises a printed circuit assembly having a transimpedance amplifier including grounded guard traces;
(xii) the signal processing module comprises a printed circuit assembly formed of surface mount components, e.g., substantially free of secondary hand soldered high gain components;
(xiii) the signal processing module comprises a printed circuit assembly comprising a thermally conductive excitation source, optionally wherein the thermally conductive excitation source is in thermal communication, e.g., thermal contact, with a thermal sink, optionally wherein the sensing system does not include a reference signal detector; and/or
(xiv) the signal processing module is configured to operate with reduced parasitic current, e.g., reduced interference, dark currents, or noise, associated with the detection and/or amplification of at least one of the first signal and the second signal.

48-66. (canceled)

67. The instrument of claim 1, wherein:
(i) the sensing system is constructed and arranged to form a measurement chamber between a sample-facing surface of each sensor unit and a sensor-facing surface of at least one well (e.g., each well) when the motion actuator assembly is deployed to position each sensor unit in fluid communication with the sample within the corresponding well, wherein evaporation of the sample, flow of the sample, or diffusion of a component, e.g., analyte, of the sample out of the measurement chamber is impaired, optionally wherein the sample temperature control element is configured to control temperature of samples within each measurement chamber;
(ii) the sensing system comprises one or more optical sensors, e.g., photoluminescence sensors;
(iii) the sensing system comprises one or more electrochemical sensors;
(iv) the sensing system is configured to generate a signal in response to rate of change of an analyte proportional to O₂ content in the sample, e.g., generates a signal proportional to oxygen consumption rate (OCR) of the sample;
(v) the sensing system is configured to generate a signal in response to rate of change of an analyte proportional to pH value in the sample, e.g., generates a signal proportional to extracellular acidification rate (ECAR) and/or proton efflux rate (PER) of the sample;
(vi) the sensing system is configured to generate a signal in response to one or more electrochemical property of the sample, e.g., impedance; and/or
(vii) the signal processing module is operatively connected to a computing network or computer device programmed to calculate one or more of mitochondrial respiration, glycolysis, adenosine triphosphate (ATP) production rate, and mitochondrial toxicity (mitotox) index value of the sample responsive to one or more of the first signal and the second signal, optionally wherein:
(a) wherein the signal processing module is operatively connected to a cloud-based computing network, and/or
(b) the signal processing module is operatively connected to a data storage module storing historical values for the first signal and the second signal, further optionally wherein the data storage module is a local memory storage device or the data storage module is a cloud-based memory storage device.

68-78. (canceled)

79. The instrument of claim 1, wherein:
(i) the array of sensor units comprises Xₛ₁ sensor units, and Xₛ₁ is equal to or greater than 1, 6, 8, 12, 24, 36, 48, 64, 72, 96, 192, or 384;
(ii) the array of injectors comprises Xₛ₂ injectors, and Xₛ₂ is equal to or greater than 1, 6, 8, 12, 24, 36, 48, 64, 72, 96, 192, 384, 768, or 1536;
(iii) the instrument has a ratio of sensor units Xₛ₁ to injectors Xₛ₂ of 1:1 to 1:384, e.g., 1:1, 1:2, 1:3, 1:4, 1:8, 1:12, 1:24, 1:36, 1:48, 1:64, 1:72, 1:96, 1:192, or 1:384;
(iv) the instrument has a ratio of injectors Xₛ₂ to sensor units Xₛ₁ of 1:1 to 1:384, e.g., 1:1, 1:2, 1:3, 1:4, 1:8, 1:12, 1:24, 1:36, 1:48, 1:64, 1:72, 1:96, 1:192, or 1:384;
(v) each sensor unit of the array of sensor units is configured to generate one or more of the first signal and the second signal independently;
(vi) each sensor unit of the array of sensor units is configured to generate the first signal and the second signal concurrently;
(vii) the instrument further comprises a light source, e.g., a fluorescent light, light emitting diode (LED), or laser, configured to excite a sensor of the sensor unit to generate one or more of the first signal and the second signal, optionally wherein:
(a) the light source is configured to produce a reference signal, wherein fluctuations in intensity from the light source are corrected proportionally to drift by monitoring the reference signal produced by the light source, and/or
(b) the light source is positioned on a thermally conductive printed circuit assembly configured to minimize drift from the light source, optionally wherein the thermally conductive printed circuit assembly is formed of a material configured to minimize drift generated by heat-induced fluctuations from the light source by at least 20%, e.g., at least 15%, 10%, 5%, or 1%;
(viii) the instrument further comprises an electric motor configured to actuate the motion actuator assembly, e.g., one or more of the x-axis actuator assembly, the z-axis actuator assembly, and the y-axis actuator assembly;
(ix) the instrument further comprises a stall sensing module programmed to generate a notification signal, and optionally pause a protocol, e.g., halt motor movement, if a predetermined protocol step is not within a predetermined time interval;
(x) the instrument further comprises a proximity sensor configured to generate a notification signal, and optionally pause a protocol, if a component is positioned within a predetermined distance from another component, e.g., a sensor unit within a predetermined distance from a corresponding well of the sample carrier;
(xi) the instrument further comprises a proximity sensor configured to generate a notification signal, and optionally pause a protocol, if the opening on the side wall of the housing is ajar and/or external light is detected within the housing;
(xii) the instrument has an OCR detection range of 2000 pmol/min to 0.01 pmol/min, e.g., 700 pmol/min to 0.01 pmol/min, e.g., 50 pmol/min to 0.01 pmol/min;
(xiii) the instrument has a lower OCR detection limit of less than 50 pmol/min, e.g., less than 40 pmol/min, 30 pmol/min, 20 pmol/min, 10 pmol/min, 5 pmol/min, 3 pmol/min, 1 pmol/min, 0.1 pmol/min, or 0.01 pmol/min;
(xiv) the instrument further comprises an optical module positioned to image or scan one or more samples within the array of wells of the sample carrier, optionally wherein the optical module is operatively connected to the computer, optionally wherein the computer is configured to display and/or record the image or scan of the samples in real time;
(xv) the instrument further comprises a transfer module formed of a multiplexed fiber optic material configured to transfer optical signals from the array of sensor units to the signal processing module, optionally wherein:
(a) the transfer module is configured to transfer one or more of excitation, reference, and emission optical signals,
(b) the transfer module is configured to directly interface with one or more sensor units, and/or
(c) the sensing system comprises a homogenized fiber optic wave guide optically connected to the transfer module, optionally wherein the homogenized fiber optic wave guide is configured to uniformly distribute light onto one or more sensor units;
(xvi) an environmental control module configured to control an environment of samples within at least one well (e.g., each well) of the sample carrier, e.g., configured to control environmental gas and/or relative humidity (RH), optionally wherein:
(a) the environmental control module is configured to control one or more of N₂, O₂, and CO₂ concentration of the gas surrounding the samples,
(b) the environmental control module comprises a source of a gas, e.g., one or more of N₂, O₂, and CO₂, fluidly connected to the sample carrier,
(c) the environmental control module forms a controlled environment zone which comprises the array of wells of the sample carrier, further optionally wherein the controlled environment zone is formed in a sealed container, e.g., hermetically sealed container; and/or
(xvii) the instrument is configured for use within a gas-controlled environment.

80-105. (canceled)

106. A method of using the analytical instrument of claim 1, comprising:
loading a sample carrier comprising one or more cell samples, each sample disposed within a corresponding well of the sample carrier, into the analytical instrument;
optionally wherein:
(a) the cell samples comprise live cells, or
(b) loading the sample carrier comprises fixing the sample carrier onto the stage.

107-108. (canceled)

109. A method of analyzing a cell sample, comprising:
providing the analytical instrument of claim 1;
loading a sample carrier comprising one or more cell samples, each sample disposed within a corresponding well of the sample carrier, into the analytical instrument;
obtaining a first plurality of values from signals in response to the first analyte, e.g., at least one analyte proportional to O₂ content, each signal generated by a corresponding sensor unit of the sensing system;
optionally, obtaining a second plurality of values from signals in response to the second analyte, e.g., at least one analyte proportional to pH value, each signal generated by the corresponding sensor unit of the sensing system; and
processing the first plurality of values;
optionally, processing the second plurality of values;
thereby analyzing the cell sample.

110. The method of claim 109, wherein:
(i) the method further comprises controlling the temperature of samples within at least one well (e.g., each well) of the sample carrier to be within the predetermined amount of the sample within another well of the sample carrier;
(ii) the sample carrier is loaded into the controlled temperature zone and/or wherein controlling the temperature of samples includes forming the controlled temperature zone;
(iii) the method further comprises controlling temperature of the sensing system;
(iv) the method further comprises dispensing a target agent into each sample within the array of wells of the sample carrier, optionally wherein the method further comprises:
(a) loading the target agent into the dispensing system of the analytical instrument, and/or
(b) controlling temperature of the target agent;
(v) the same sample is present within at least one well (e.g., each well) of the array of wells of the sample carrier;
(vi) a first sample is present in a first well of the array of wells of the sample carrier and a second sample is present in a second well of the array of wells of the sample carrier, optionally wherein the first sample is a test sample and the second sample is a control;
(vii) the sample comprises live cells;
(viii) the sample comprises one or more of loose cells, cell constructs, loose tissue, tissue constructs, organelles, enzymes, cell products or byproducts, and conditioned medium;
(ix) the sample comprises mammalian cells or tissue;
(x) the sample comprises non-mammalian cells or tissue;
(xi) the sample comprises single-celled organisms, e.g., microorganisms;
(xii) the sample comprises whole animal model tissues, e.g., zebrafish, C. elegans, drosophila;
(xiii) the sample comprises whole plant model tissues or plant model cells;
(xiv) the first analyte is proportional to O₂ content;
(xv) the second analyte is proportional to pH value;
(xvi) the first value and the second value are obtained independently;
(xvii) the first value and the second value are obtained concurrently;
(xviii) the method further comprises obtaining an image or scan of the samples during or after the analysis;
(xix) the method further comprises measuring one or more electrochemical property, e.g., impedance, of the samples during or after the analysis;
(xx) the method further comprises obtaining or calculating a mitochondrial toxicity (mitotox) index value of the samples during or after the analysis; and/or
(xxi) the method further comprises controlling the environment of samples within at least one well (e.g., each well) of the sample carrier, e.g., controlling environmental gas and/or relative humidity (RH), optionally wherein controlling the environment includes controlling one or more of N₂, O₂, and CO₂ concentration of the gas surrounding the samples.

111-134. (canceled)
